(19) United States
(12) Patent Application Publication
(10) Pub. No.: US 2024/0024494 A1
(43) Pub. Date: Jan. 25, 2024

(54) SMALL-MOLECULE CONJUGATE AND USE THEREOF

(71) Applicant: SHANGHAI MEWSGEN BIOTECHNOLOGY CO., LTD, Shanghai (CN)

(72) Inventor: Juan WANG, Shanghai (CN)

(21) Appl. No.: 18/026,052

(22) PCT Filed: Aug. 30, 2021
(86) PCT No.: PCT/CN2021/115248

§ 371 Date: Jul. 6, 2023

(30) Foreign Application Priority Data
Sep. 11, 2020 (CN) 202010952864.9

(51) Int. Cl. A61K 47/64; A61K 47/54; A61P 35/00

(52) U.S. Cl. CPC A61K 47/64; A61P 35/00; A61K 47/554

(57) ABSTRACT

The present disclosure discloses a small-molecule conjugate or a pharmaceutical salt thereof, wherein a structure of the small-molecule conjugate is shown in a general formula I:

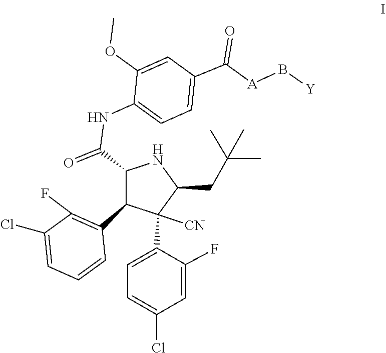

A is a spacer linking group;
B is a releasable linking group; and
Y is a drug.

The present disclosure further provides use of the small-molecule conjugate or the pharmaceutical salt thereof in the preparation of an antitumor drug.

### CROSS-REFERENCE TO RELATED APPLICATIONS

This Application is a national stage application of PCT/CN2021/115248. This application claims priorities from PCT Application No. PCT/CN2021/115248, filed Aug. 30, 2021, and from the Chinese patent application 202010952864.9 filed Sep. 11, 2020, the content of which are incorporated herein in the entirety by reference.

### TECHNICAL FIELD

The present disclosure belongs to the technical field of medicines and particularly relates to a small-molecule conjugate, and a pharmaceutical composition and use thereof.

### BACKGROUND

p53 gene is one of the most widely studied tumor suppressor genes. p53 protein is inactivated due to mutation in p53 in about 50% of human tumor cells. Normal biological functions of the p53 protein are also lost due to an interaction between some proteins in human body and the p53 protein. Murine double mimute 2 (MDM2) is one of the most important regulatory proteins of p53. When the two proteins are bound, the p53 protein will be degraded. An antitumor drug with a brand-new mechanism is designed by taking a p53-MDM2 protein-protein interaction as a target is a hotspot in the research and development field of antitumor drugs (Duffy M. J., et al. Semin Cancer Biol. 2020, S1044-579X, 30160).

Since the first small-molecule p53-MDM2 inhibitor was reported, researchers discovered multiple backbone small-molecule inhibitors, wherein RG7112, idasanutlin, AMG-232, SAR405838, NVP-CGM097, DS-3032b, APG115, HDM201, etc. were sequentially subjected to clinical trial studies (Liu Y, et al. Eur J Med Chem. 2019, 176, 92). A design of small-molecule inhibitors based on a protein-protein interaction has a great difficulty. Until now, no p53-MDM2 small molecular inhibitors have been marketed. Most rapidly advancing is an oral MDM2 protein inhibitor idanasutlin developed by the Roche, which has entered a phase III clinical study, but unfortunately the phase III clinical trial scheme for idanasutlin in combination with a chemotherapeutic drug cytarabine was declared terminated by the Roche in April 2020.

Despite the great difficulty existing in developing antitumor drugs targeting p53-MDM2, researchers have been working on finding a highly active small-molecule inhibitor and designing a therapeutic regimen for combination with chemotherapeutic drugs, immune checkpoint inhibitors, etc.

### SUMMARY

A first object of the present disclosure is to provide a small-molecule conjugate which structurally comprises an MDM2 inhibitor, a linking group, and a chemotherapeutic drug.

A second object of the present disclosure is to provide use of the small-molecule conjugate in preparing an antitumor drug.

To achieve the above objects, the technical solutions of the present disclosure are as follows:

In a first aspect, the present disclosure provides a small-molecule conjugate or a pharmaceutical salt thereof. A structure of the small-molecule conjugate is shown in a general formula I:

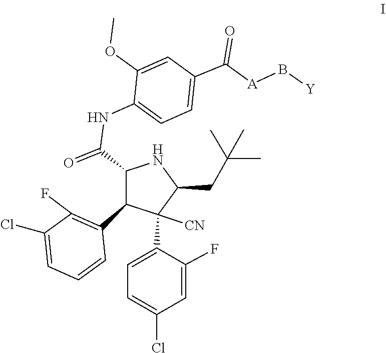

A is a spacer linking group;
B is a releasable linking group; and
Y is a drug, preferably a chemotherapeutic drug;
the A is selected from:

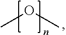

wherein n is 1-10,

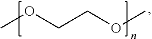

wherein n is 1-10, sugar (such as glucose), alkylene (such as —(CH₂)ₙ—, wherein n is 1-10), 1-alkylene succinimide-3-yl (such as

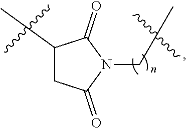

wherein n is 1-10), 1-(carbonylalkyl)succinimide-3-yl (such as

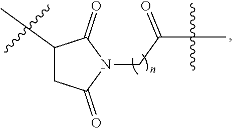

wherein n is 1-10), or a combination thereof; wherein the A may be substituted by at least one substituent selected from alkyl, alkoxy, alkoxyalkyl, hydroxyl, hydroxyalkyl, amino, aminoalkyl, alkylaminoalkyl, dialkylaminoalkyl, mercaptoalkyl, alkylthioalkyl, aryl, substituted aryl, aralkyl, substituted aralkyl, heteroaryl, substituted heteroaryl, carboxyl, carboxyalkyl, carboxylate alkyl ester, guanidinoalkyl, or carbonyl or acylamino or acylaminoalkyl substituted with an amino acid and a derivative thereof, and a peptide;
the B comprises at least one linking group formed by an amino acid selected from a natural amino acid or a non-natural a amino acid, preferably comprises a peptide linking group formed by 1-20 amino acids, and more preferably, comprises at least a linking group of a dipeptide, a tripeptide, a tetrapeptide, a pentapeptide, a hexapeptide, a heptapeptide, an octapeptide, a nonapeptide, a decapeptide, an undecapeptide, and a dodecapeptide formed by aspartic acid, arginine, cysteine, citrulline, valine, glycine, phenylalanine, alanine, methionine, lysine, and a combination thereof; or
the B is a linking group of a cleavable bond under a physiological condition (a pH labile bond, an acid labile bond, an oxidation labile bond, or an enzyme labile bond). As described in the present disclosure, the cleavable bond may link two adjacent atoms within a releasable linking group, and/or link other linking groups and/or the drug Y at either end or both ends of the releasable linking group. When such cleavable bond links two adjacent atoms within the releasable linking group, after the bond is cleaved, the releasable linking group is cleaved into two or more fragments. Instability of the cleavable bond may be adjusted, for example, by substitution changes at or near a cleavable site, including, for example, an a branching adjacent to a cleavable disulfide bond, homologization to form a part of a hydrolyzable ketal or an alkoxy of an acetal, and the like.

Preferably, the B is selected from one of the following structures or a combination thereof:

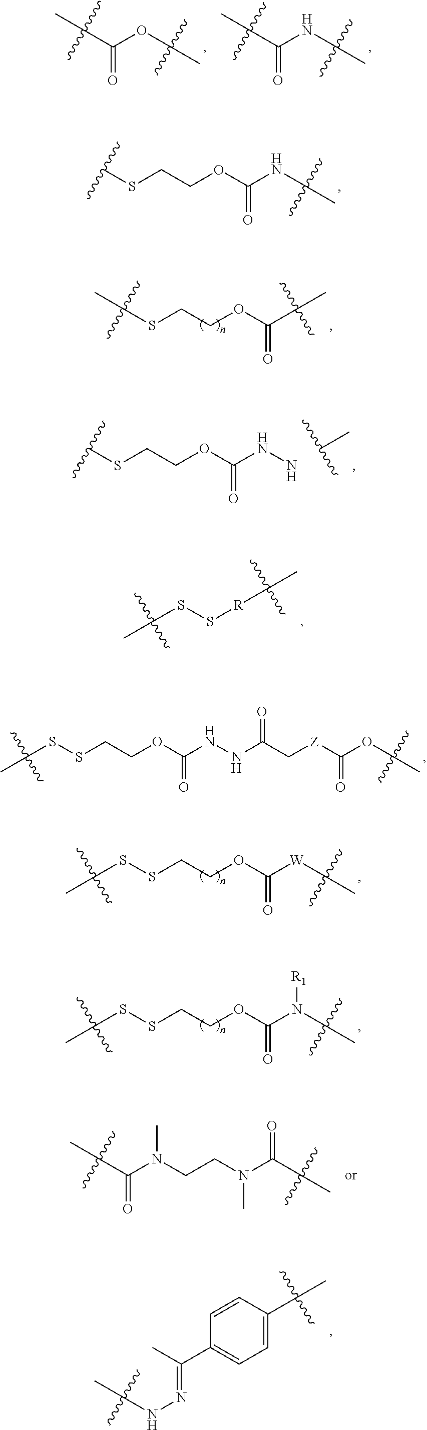

wherein n is 0, 1, 2, 3, or 4; R is —(CH₂)n— and —OOCCH₂—, and n is 1-10;
Z is —O—, —CH₂— or —NH—; W is O or S;
R₁ is hydrogen, C₁-C₁₀ alkyl, and optionally a substituted acyl or an amino protecting group (such as Boc, Fmoc, Cbz, benzyl, triphenylmethyl, etc).

The Y is a chemotherapeutic drug comprising a pharmaceutically active compound and the drug may be linked to the B through an active group such as hydroxyl and amino The pharmaceutically active compound may be a drug known in the art or a derived form thereof. The drug is cytotoxic, increases tumor permeability, inhibits tumor cell proliferation, promotes apoptosis, decreases an anti-apoptotic activity in cells, and enhances cell necrosis. The drug suitable for the present disclosure includes but is not limited to a hormone, an antibiotic, an antimicrobial drug, an antiviral drug, and an anticancer drug. Examples of the cytotoxic drug include: a cyclopropylbenzolelindolone analogue or a derivative thereof, an open-ring-cyclopropyl benzo lel indolone analogue, an O-Ac-open-ring-cyclopropylbenzolelindolone analogue or a derivative thereof, dolastatins, auristatins, tubuyysin, combretastatins, maytansine, DM1, epothilones, paclitaxel and a derivative thereof, vinblastine and an analogue thereof, camptothecin and an analogue thereof, colchicine and an analogue thereof, daunorubicin, rhizomycin, cyclophosphamide, methotrexate, bleomycin, temsirolimus, mitomycins, a microtubule inhibitor, pyrrolobenzodiazepine (PBD) dimers, cyclopropylbenzo[e]indolone, calichemicin, arenobufagin and a derivative thereof, and bufalin and a derivative thereof. Other drugs suitable for the present disclosure include a macrolide antitumor drug, a chemotherapeutic drug such as an alkylating agent of nitrogen mustard, nitrosourea, busulfan, chlorambucil, carboplatin, cisplatin and other platinum compounds, an antimetabolite such as cytarabine, a purine analogue, a pyrimidine analogue and penicillin, cephalosporin, vancomycin, erythromycin, clindamycin, rifampin, chloramphenicol, an aminoglycoside antibiotic, acyclovir, trifluridine, ganciclovir, zidovudine, amantadine, ribavirin, gemcitabine, and any recognized antimicrobial compound in the art.

Further, the Y of the present disclosure is temsirolimus, an open-ring-cyclopropyl benzolelindolone analogue, pyrrolobenzodiazepine (PBD) dimers, calichemicin, camptothecin and an analogue thereof, paclitaxel and a derivative thereof, vinblastine and an analogue thereof, dolastatins, auristatin, tubulysin, combretastatin, maytansine, DM1, epothilones, mitomycins, daunorubicin compounds, arenobufagin and a derivative thereof, or bufalin and a derivative thereof.

Furthermore, the Y is the open-ring-cyclopropyl benzolelindolone analogue, the pyrrolobenzodiazepine (PBD) dimers, the calichemicin, the camptothecin, 7-ethyl-10-hydroxycamptothecin (SN-38), exatecan and a derivative thereof, 7-cyclohexyl-21-fluorocamptothecin, DAVLBH, tubulysin B, MMAE, MMAF, an MMAF derivative, DM1, the paclitaxel and a derivative thereof, epothilone B, mitomycin C, the arenobufagin and a derivative thereof, the bufalin and a derivative thereof, the vincristine, daunorubicin, doxorubicin or epirubicin.

Furthermore, preferably, an A-B is selected from one of the following structures:

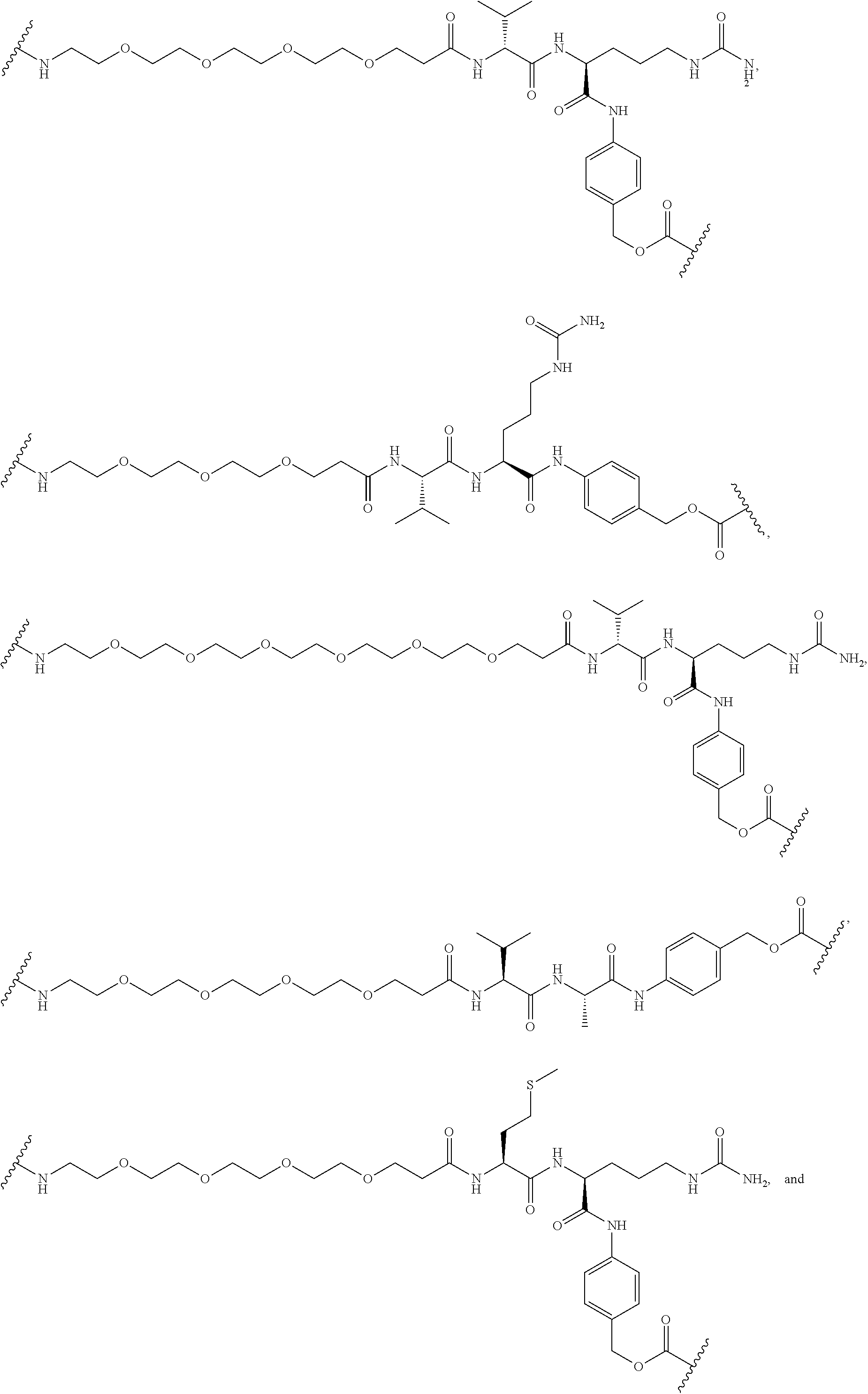

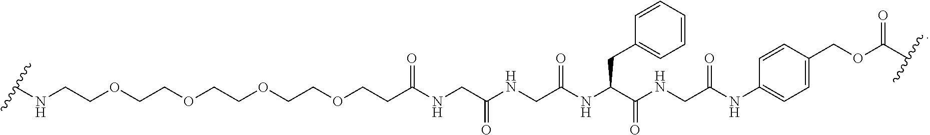 [SMILES: *NCCOCCOCCOCCOCCC(=O)NCC(=O)NCC(=O)N[C@@H](Cc1ccccc1)C(=O)NCC(=O)Nc1ccc(COC(*)=O)cc1]

Preferably, the small-molecule conjugate is selected from one of the following structures:

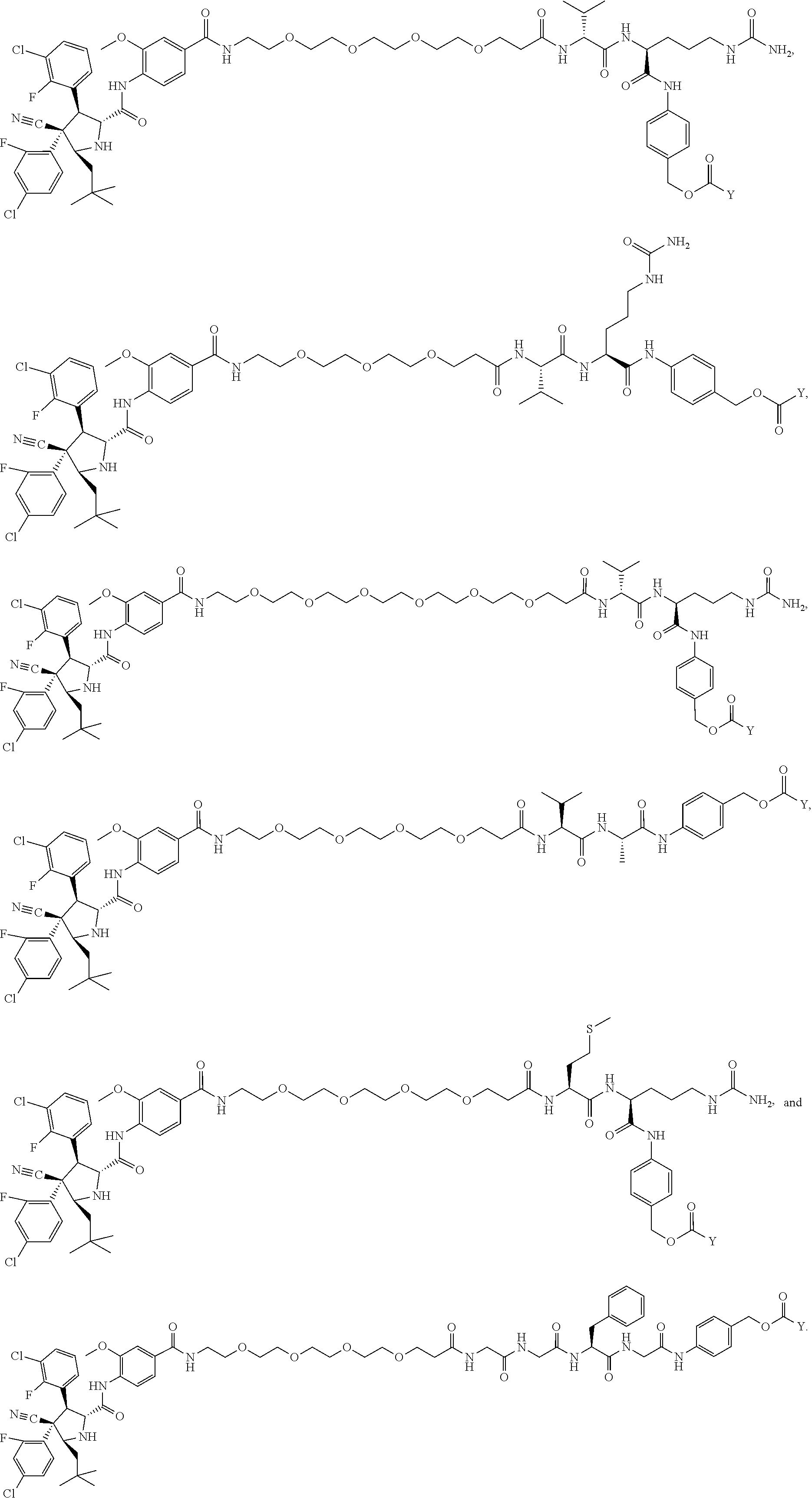

The Y is selected from

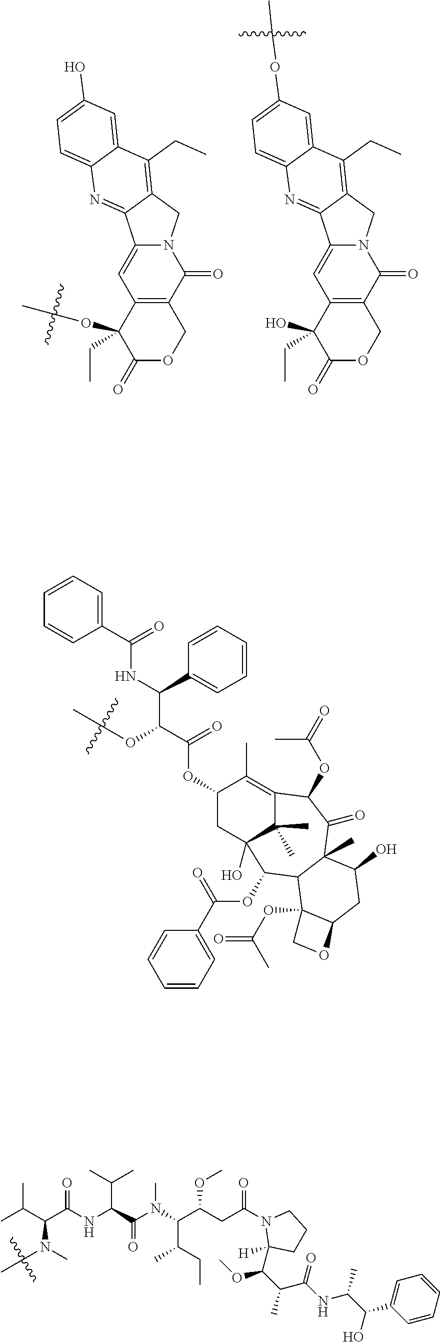

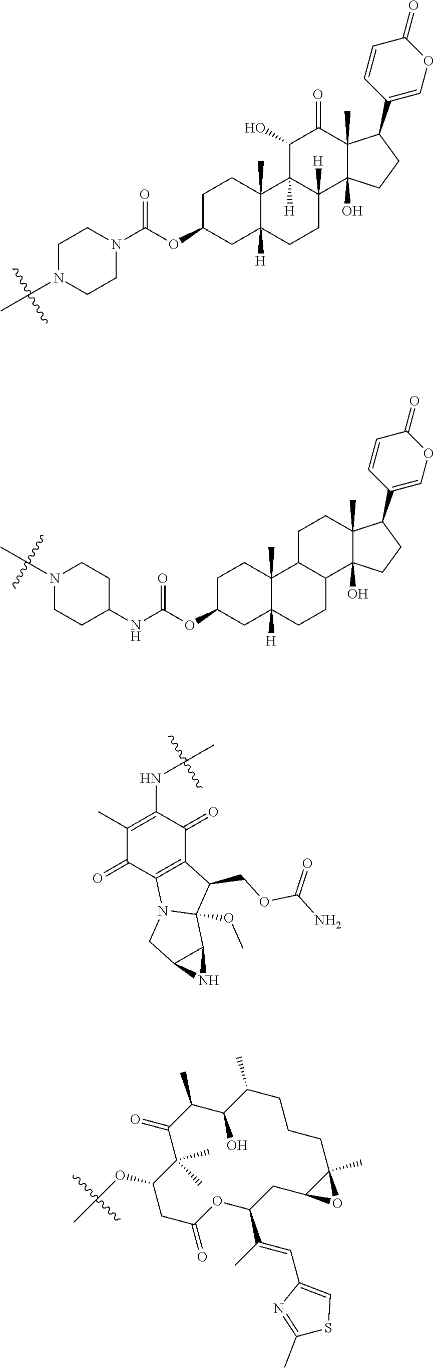

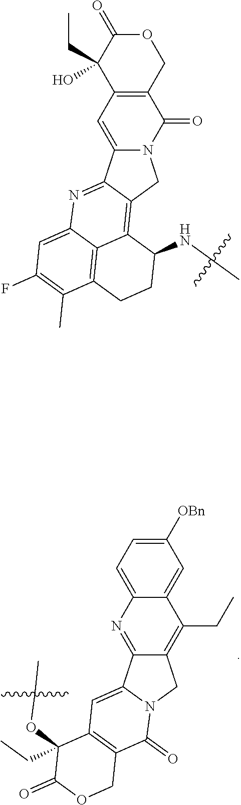 [SMILES: *N[C@H]1CCc2c(C)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccc(OCc3ccccc3)cc1c2CC]

Most preferably, the small-molecule conjugate is selected from one of the following structures:

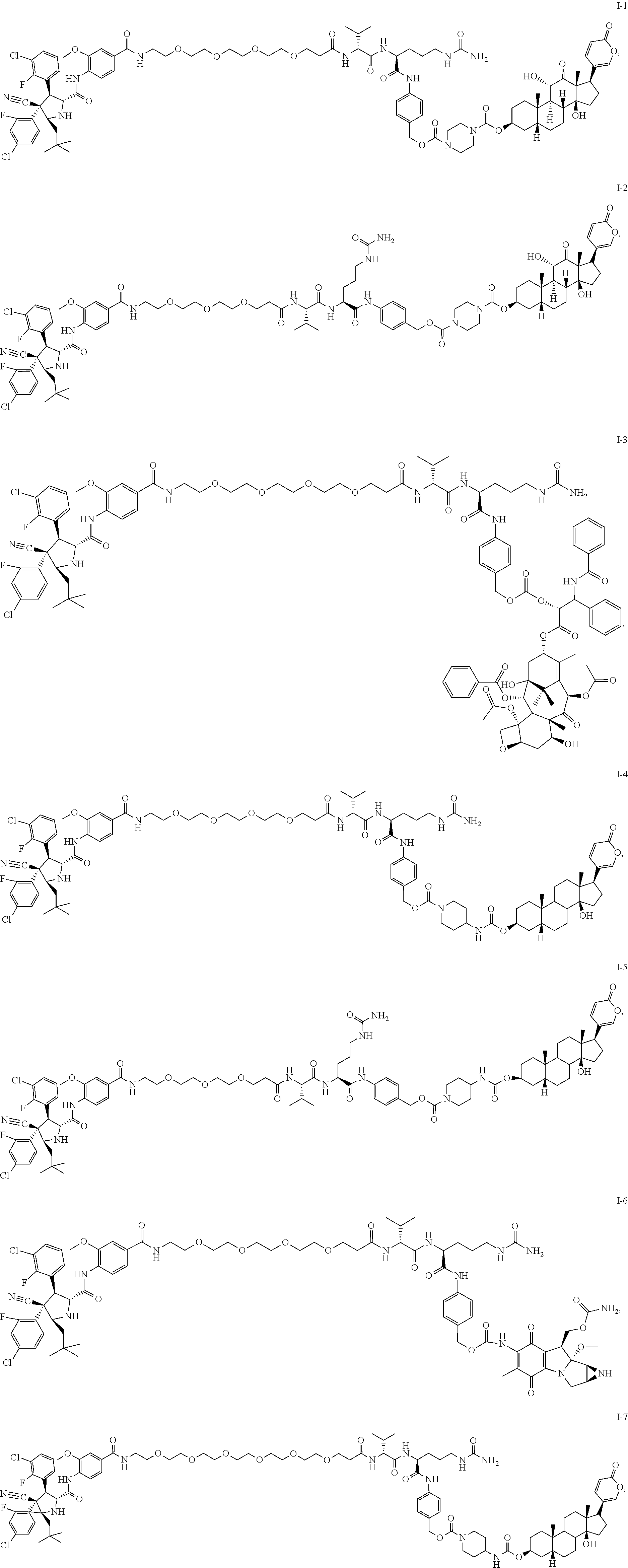

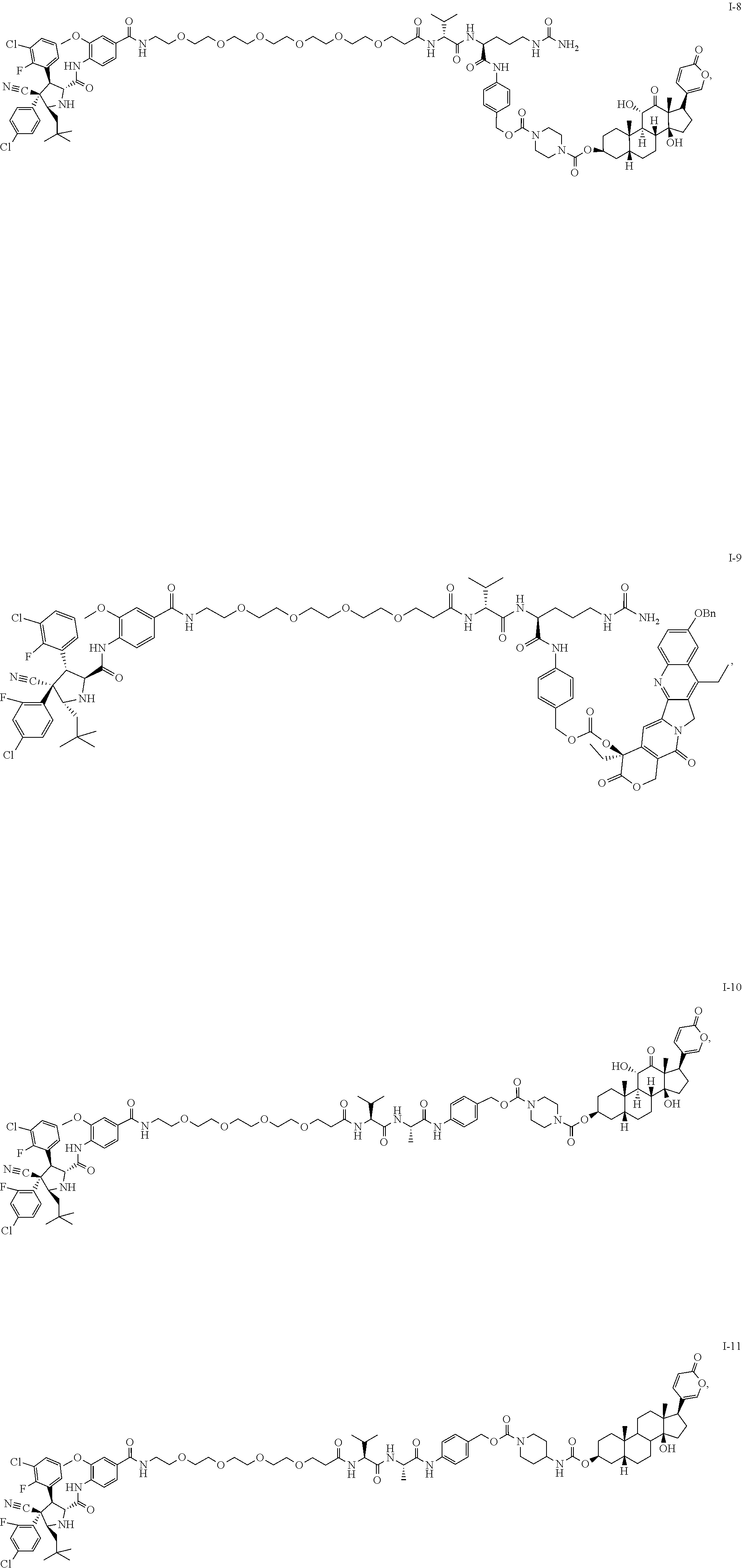

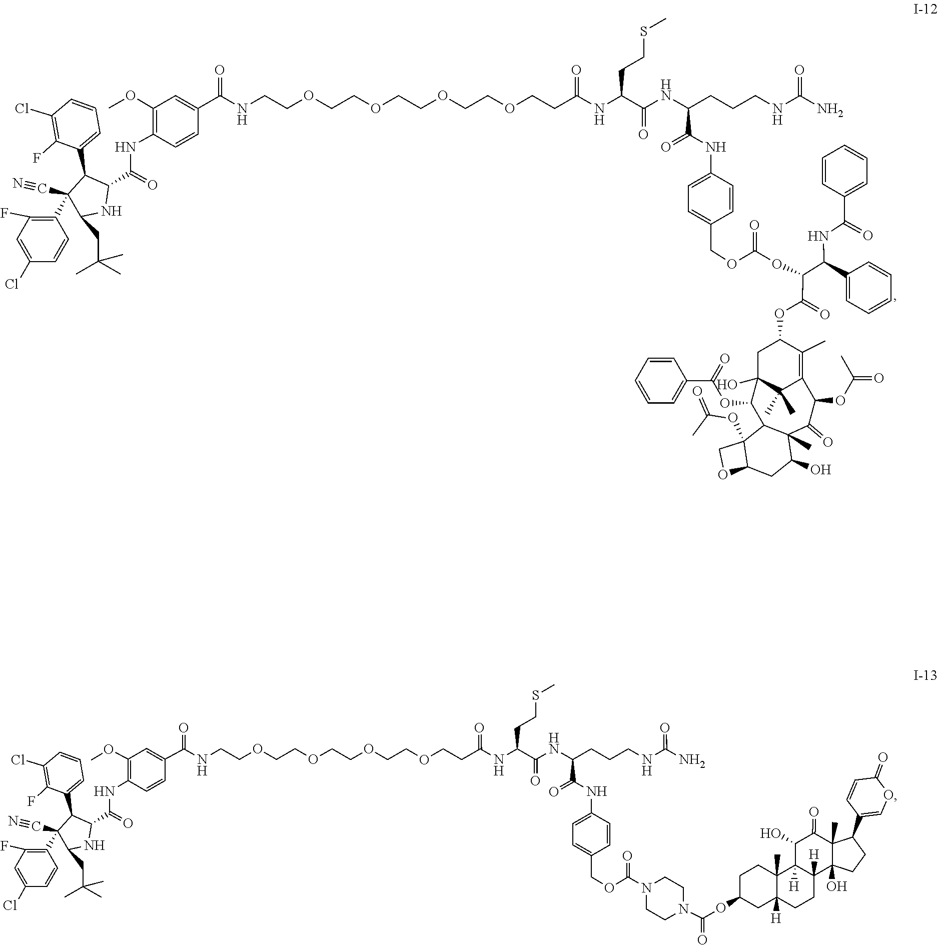

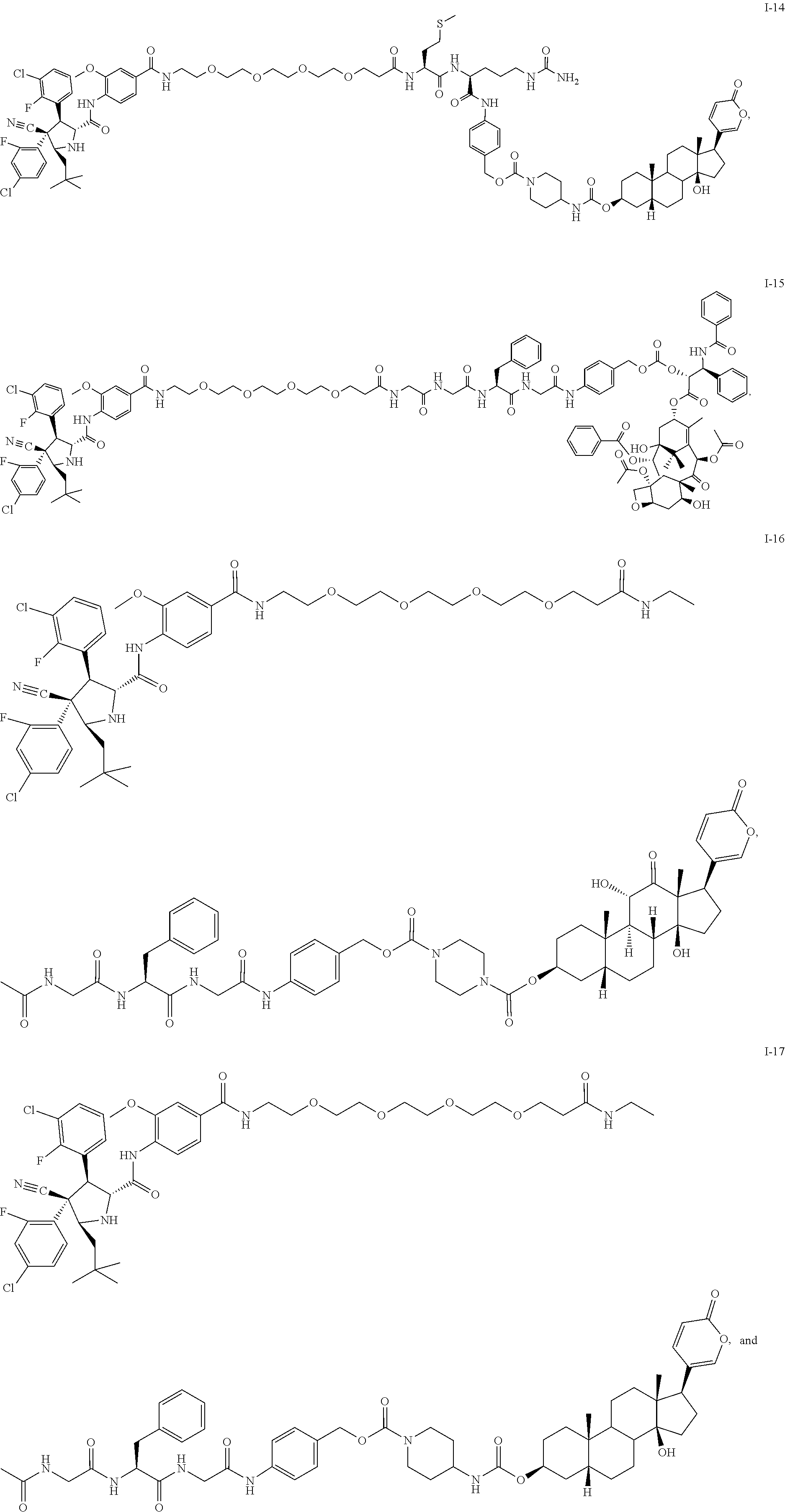

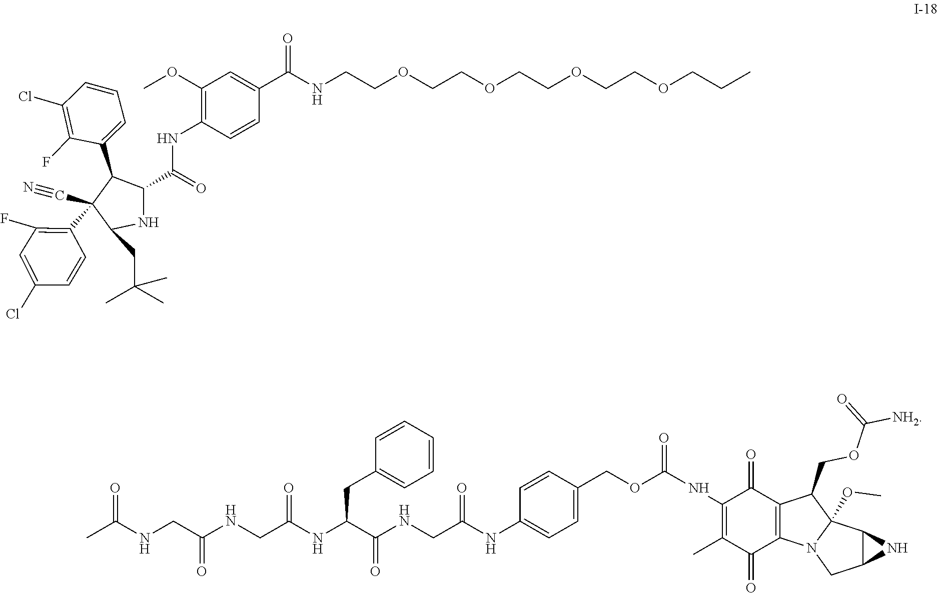

The small-molecule conjugate of the present disclosure may be prepared into a pharmaceutical salt form according to a conventional method.

The pharmaceutical salt of the small-molecule conjugate is a salt formed by a pharmaceutically acceptable inorganic acid and organic acid, wherein the preferred inorganic acid comprises: hydrochloric acid, hydrobromic acid, phosphoric acid, nitric acid, and sulfuric acid; the preferred organic acid comprises: formic acid, acetic acid, propionic acid, succinic acid, naphthalenedisulfonic acid (1, 5), asiatic acid, carbenoxolone, glycyrrhetinic acid, oleanolic acid, maslinic acid, ursolic acid, corosolic acid, betulinic acid, masticinic acid, oxalic acid, tartaric acid, lactic acid, salicylic acid, benzoic acid, valeric acid, diethylacetic acid, malonic acid, succinic acid, fumaric acid, pimelic acid, adipic acid, maleic acid, malic acid, sulfamic acid, phenylpropionic acid, gluconic acid, ascorbic acid, nicotinic acid, isonicotinic acid, methanesulfonic acid, ethanesulfonic acid, p-toluenesulfonic acid, citric acid, and amino acids.

A second aspect of the present disclosure provides use of the small-molecule conjugate or the pharmaceutical salt thereof in the preparation of an antitumor drug.

A pharmacological activity of the compound of the present disclosure enables the compound to be used in the preparation of drugs for resisting tumors, treating cardiovascular diseases, and resisting inflammation and nervous system diseases (Rialdi A, et al, Science, 2016, 352, 6289; Pan P, J Med Chem, 2018, 61, 8613).

The small-molecule conjugate of the present disclosure has an antitumor activity. The tumor includes cancers occurring in esophagus, stomach, intestine, rectum, oral cavity, pharynx, larynx, lungs, colon, breast, uterus, endometrium, ovary, prostate, testis, bladder, kidneys, liver, pancreas, bone, connective tissue, skin, eyes, brain, and central nervous system, thyroid cancer, leukemia, Hodgkin's disease, lymphoma, myeloma, and the like, especially esophageal cancer, gastric cancer, colon cancer, lung cancer, breast cancer, osteosarcoma, hepatocarcinoma, and brain glioma.

A third aspect of the present disclosure provides a pharmaceutical composition comprising the small-molecule conjugate or the pharmaceutical salt thereof as a pharmaceutical active ingredient; or the pharmaceutical composition further comprises at least one therapeutic agent, such as a chemotherapeutic agent, an immune checkpoint inhibitor, an inflammation regulator, an anti-hypercholesteremia agent, an anti-infection agent, or a radiotherapy drug and the like (Fang D., et al J Immunother Cancer, 2019, 7,327; Yi H. et al. J Exp Clin Cancer Res, 2018, 37, 97).

The pharmaceutical composition may be in a solid form or in a liquid form, and may further be used for the preparation of the following drugs: drugs for treating cardiovascular and cerebrovascular diseases, inflammation, and nervous system diseases.

A fourth aspect of the present disclosure provides a pharmaceutical preparation comprising the small-molecule conjugate or the pharmaceutical salt thereof.

The small-molecule conjugate of the present disclosure may be prepared into a pharmaceutical preparation together with a conventional pharmaceutic adjuvant in pharmaceutics.

The pharmaceutical preparation comprises a small-volume injection, a medium-volume injection, a large-volume injection, a powder injection, an emulsion for injection, a tablet, a pill, a capsule, an unguent, a cream, a patch, a liniment, a powder, a spray, an implant, a drop, a suppository, an ointment, various nano-preparations, and a liposome; and the corresponding liposome is mainly prepared into the above-mentioned injection.

The term “alkyl” used in the present disclosure refers to a saturated straight-chain or branched-chain monovalent hydrocarbyl having one to twelve carbon atoms, wherein the alkyl may be optionally substituted independently with one or more substituents described below. Examples of the alkyl include but are not limited to methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, cyclopropyl, cyclobutyl, cyclopentyl, cyclohexyl, cycloheptyl, and cyclooctyl.

The term “cycloalkyl” used in the present disclosure refers to a monovalent non-aromatic saturated or partially saturated cyclic hydrocarbon atomic group having three to ten carbon atoms. Examples of the cycloalkyl include but are not limited to cyclopropyl, cyclobutyl, cyclopentyl, 1 -cyclopentyl- 1 -enyl, 1 -cyclopentyl-2-enyl, 1 -cyclopentyl-3 -enyl, cyclohexyl, 1-cyclohexyl-1-enyl, 1-cyclohexyl-2-enyl, 1-cyclohexyl-3-enyl, cyclohexadienyl, cycloheptyl, cyclooctyl, cyclononyl, cycloundecyl, and cyclododecyl. The term “cycloalkyl” also includes a polycyclic (e.g., bicyclic and tricyclic) cycloalkyl structure, wherein the polycyclic structure optionally includes a saturated or partially unsaturated cycloalkyl or heterocyclyl or aryl or heteroaryl ring fused saturated or partially unsaturated cycloalkyl. Bicyclic carbocycles having 7 to 12 atoms may be arranged, for example, as bicyclo [4, 5], [5, 5], [5, 6] or [6, 6] systems or as bridged systems, for example bicyclo[2.2.1]heptane, bicyclo [2.2.2]heptaoctane, and bicyclo [3 .2.2]nonane.

The term “heteroalkyl” used in the present disclosure refers to a saturated straight-chain or branched-chain monovalent hydrocarbyl having one to twelve carbon atoms, wherein at least one carbon atom is substituted with a heteroatom selected from nitrogen, oxygen, and sulfur, and the group may be a carbon group or a heteroatom group (i.e., the heteroatom may occur in a middle or at an end of the group). The heteroalkyl may be optionally substituted independently with one or more substituents described in the present disclosure. The term “heteroalkyl” also includes alkoxy and heteroalkoxy.

The term “heterocyclyl” used in the present disclosure refers to a saturated or partially unsaturated carbocyclic group having 3 to 8 ring atoms, wherein at least one ring atom is a heteroatom independently selected from nitrogen, oxygen, and sulfur, the remaining ring atoms are carbon atoms, wherein one or more ring atoms may be optionally independently substituted with one or more substituents as described below. The group may be a carbon group or a heteroatom group. The term “heterocyclyl” further includes heterocyclylalkoxy and further includes a group in which the heterocyclyl is fused to a saturated, partially unsaturated, or fully unsaturated (i.e., aromatic) carbocyclic or heterocyclic ring. Examples of the heterocyclyl include but are not limited to pyrrolidinyl, tetrahydrofuryl, dihydrofuranyl, tetrahydrothienyl, tetrahydropyranyl, dihydropyranyl, tetrahydrothiopyranyl, piperidinyl, morpholinyl, 4-thiomorpholinyl, thioxanyl, piperazinyl, homopiperazinyl, azacyclobutyl, oxaclobutyl, thiacyclobutyl, homopiperidinyl, oxacycloheptyl, thiepanyl, oxazepinyl, diazepinyl, thiazepinyl, 2-pyrrolinyl, 3 -pyrrolinyl, indolinyl, 2H-pyranyl, 4H-pyranyl, dioxacyclohexyl, 1,3 -dioxacyclopentyl, pyrazolinyl, dithiacyclohexyl, dihydropyranyl, dihydrothienyl, dihydrofuranyl, pyrazolidinyl, imidazolinyl, imidazolidinyl, 3-azabicyclo[3.1.0]hexane, 3-azabicyclo [4.1.0]heptanyl, azabicyclo[2.2.2]hexyl, 3H-indolyl, quinolizinyl, and N-pyridylurea. A Spiro moiety is also included within the scope of the definition. The heterocyclyl may be C-linked or N-linked as long as it is possible. For example, a group derived from pyrrole may be pyrrol-1-yl(N-linked) or pyrrol-3-yl (C-linked). In addition, a group derived from imidazole may be imidazol-1-yl(N-linked) or imidazol-3-yl(C-linked). Examples of the heterocyclyl wherein 2 ring carbon atoms are partially substituted with an oxo moiety (C═O) are isoindoline-1,3-diketo and 1,1-dioxothiomorpholinyl. The heterocyclyl of the present disclosure may be unsubstituted or substituted with various groups at one or more substitutable positions as specified.

The term “aryl” used in the present disclosure refers to an optionally substituted monocyclic or polycyclic group or ring system containing at least one aromatic hydrocarbon ring, such as, but is not limited to phenyl, naphthyl, fluorenyl, azulenyl, anthracenyl, phenanthryl, pyrenyl, biphenyl, and terphenyl.

The term “heteroaryl” used in the present disclosure refers to an optionally substituted monocyclic or polycyclic group or ring system containing at least one aromatic ring having one or more heteroatoms independently selected from nitrogen, oxygen, and sulfur. Examples of the monocyclic heteroaryl, such as, but are not limited to furyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of the bicyclic heteroaryl, such as, but are not limited to benzofuranyl, benzimidazolyl, benzisoxazolyl, benzopyranyl, benzothiadiazolyl, benzothiazolyl, benzothienyl, benzotriazolyl, benzoxazolyl, furopyridinyl, imidazopyridinyl, imidazothiazolyl, indolizinyl, indolyl, indazolyl, isobenzofuranyl, isobenzothienyl, isoindolyl, isoquinolyl, isothiazolyl, naphthyridinyl, oxazolopyridyl, phthalazinyl, pteridinyl, purinyl, pyridopyridinyl, pyrrolopyridinyl, quinolinyl, quinoxalinyl, quinazolinyl, thiadiazolopyrimidyl, and thienopyridinyl. Examples of the tricyclic heteroaryl, such as, but are not limited to acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl.

The term “arylalkyl” used in the present disclosure refers to the alkyl (as defined above) substituted with one or more aryl moieties (as defined above). Examples of the arylalkyl include aryl-C₁₋₃-alkyl, such as, but are not limited to benzyl, phenylethyl, and the like.

The term “heteroarylalkyl” used in the present disclosure refers to an alkyl moiety (as defined above) substituted with a heteroaryl moiety (as defined above). Examples of the heteroarylalkyl include 5- or 6-membered heteroaryl-C₁₋₃-alkyl such as, but are not limited to oxazolylmethyl, pyridylethyl and the like.

The term “heterocyclylalkyl” used in the present disclosure refers to an alkyl moiety (as defined above) substituted with a heterocyclyl moiety (as defined above). Examples of the heterocyclylalkyl include 5- or 6-membered heteroaryl-C₁₋₃-alkyl, such as, but are not limited to tetrahydropyranylmethyl.

The term “substituted alkyl” used in the present disclosure refers to an alkyl group wherein one or more hydrogen atoms are each independently substituted with a substituent. Typical substituents include but are not limited to, F, Cl, Br, I, CN, CF₃, OR, R, ═O, ═S, ═NR, ═N⁺(O)(R), ═N(OR), ═N⁺(O)(OR), ═N(OR), ═N⁺(O)(OR), ═N—NRR′, —C(═O)R, —C(═O)OR, —C(═O)NRR′, —NRR′, —N⁺RR′R″, —N(R)C(═O)R′, —N(R)C(═O)OR′, —N(R)C(═O)NR′R″, —SR, —OC(═O)R, —OC(═O)OR, —OC(═O)NR′R″, —OS(O)₂OR, —OP(═O)(OR)₂, —OP(OR)₂, —P(═O)(OR)₂, —P(═O)(OR)NR′R″, —S(O)R, —S(O)₂R, —S(O)₂NR, —S(O)(OR), —S(O)₂(OR), —SC(═O)R, —SC(═O)OR, ═O, and —SC(═O)NR′R″, wherein each of R, R′, and R″ is independently selected from hydrogen, deuterium, alkyl, alkenyl, alkynyl, aryl, and heterocyclyl. The alkenyl, alkynyl, allyl, saturated or partially unsaturated cycloalkyl, heteroalkyl, heterocyclyl, arylalkyl, heteroarylalkyl, heterocyclylalkyl, cycloalkylalkyl, aryl, and heteroaryl as defined above may also be similarly substituted.

The term “halogen” used herein includes fluorine, bromine, chlorine, and iodine.

By adopting the foregoing technical solutions, the present disclosure achieves the following advantages and beneficial effects.

After the compounds of the present disclosure are selectively metabolized by an enzyme highly expressed by a tumor cell, a cytotoxic drug and a p53-MDM2 small-molecule inhibitor are released, and thus a dual anti-tumor effect is achieved.

### DETAILED DESCRIPTION OF EMBODIMENTS

To more clearly illustrate the present disclosure, the present disclosure is further illustrated below in combination with the preferred examples. A person skilled in the art should understand that the content specifically described below is illustrative rather than restrictive, and should not be used to limit the protection scope of the present disclosure.

### EXAMPLE 1

### Preparation of Compound I-1

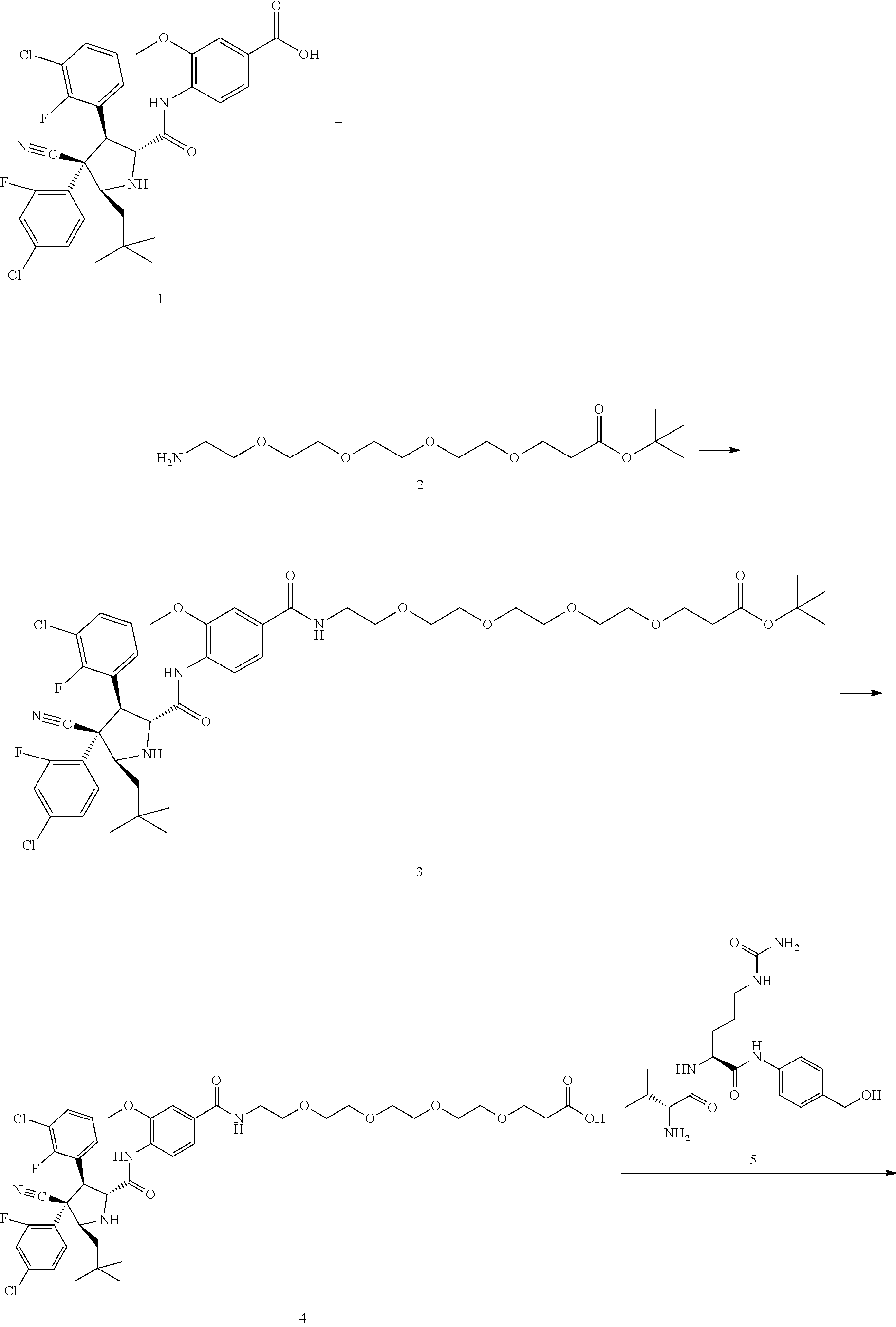

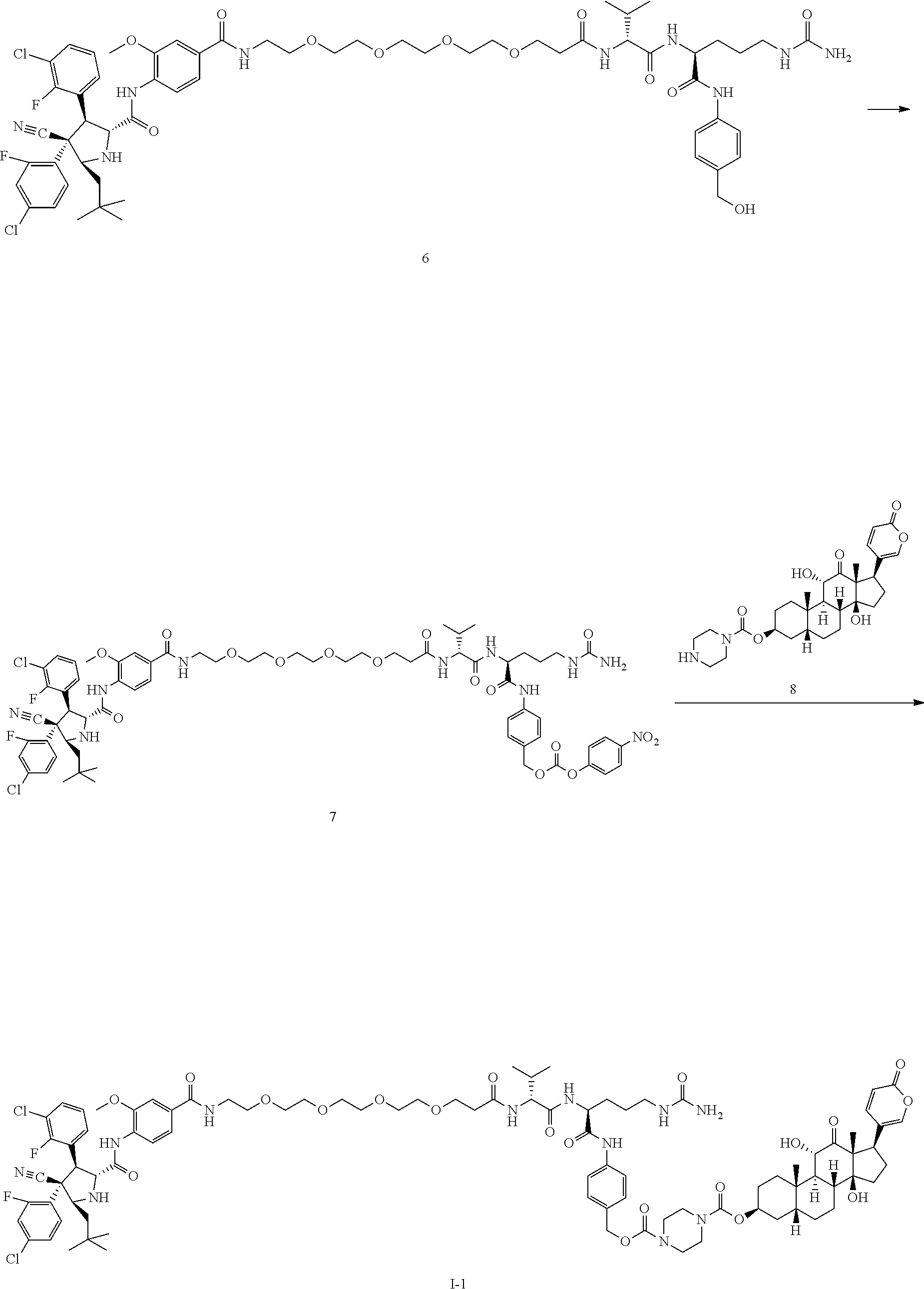

A compound 1 (250 mg, purchased from Pinghu Zhengyuan company) and 5 mL of dry DMF were added into a 25-mL flask, dissolved with stirring, and EDCI (85 mg) and HOBt (60 mg) were added, and the mixture reacted at room temperature for 30 minutes. A compound 2 (196 mg) was then added and reacted overnight at room temperature. The reaction solution was poured into 50 mL of ice water, extracted with ethyl acetate (20 mL×3), and dried over anhydrous sodium sulfate. A solvent was evaporated at a reduced pressure. The residue was separated by a column chromatography to obtain 360 mg of a colorless liquid compound 3 (dichloromethane:methanol=100:2) with a yield of 98.1%. ¹H NMR (600 MHz, DMSO-d6) δ: 10.41 (s, 1H), 8.48 (t, J=5.5 Hz, 1H), 8.32 (d, J=8.4 Hz, 1H), 7.73 (t, J=7.0 Hz, 1H), 7.61-7.51 (m, 3H), 7.49 (d, J=8.5 Hz, 1H), 7.37 (dt, J=10.3, 7.3 Hz, 3H), 4.62-4.56 (m, 2H), 4.37 (s, 1H), 3.99-3.89 (m, 4H), 3.60-3.44 (m, 16H), 3.44-3.38 (m, 2H), 2.40 (t, J=6.2 Hz, 2H), 1.68-1.61 (m, 1H), 1.38 (s, 9H), 1.27 (d, J=13.9 Hz, 1H), 0.98 (s, 9H).

The compound 3 (140 mg), 5 mL of dichloromethane, and 300 μL of trifluoroacetic acid were added to a 5-mL flask, and reacted overnight at room temperature. A solvent was evaporated at a reduced pressure to obtain 100 mg of a white solid compound 4 with a yield of 76.3%. ¹H NMR (600 MHz, DMSO-d6) δ: 12.16 (s, 1H), 10.42 (s, 1H), 8.49 (t, J=5.5 Hz, 1H), 8.33 (d, J=8.3 Hz, 1H), 7.74 (t, J=7.0 Hz, 1H), 7.61-7.53 (m, 3H), 7.50 (d, J=8.5 Hz, 1H), 7.38 (dt, J=8.9, 6.9 Hz, 3H), 4.61 (s, 2H), 4.38 (s, 1H), 4.01-3.89 (m, 4H), 3.59 (t, J=6.3 Hz, 2H), 3.47 (ddd, J=19.9, 17.4, 14.8 Hz, 17H), 2.44 (t, J=6.4 Hz, 2H), 1.66 (dd, J=13.7, 10.1 Hz, 1H), 0.99 (s, 9H).

The compound 4 (400 mg), 5 mL of DMF, and 242 μL of DIPEA were added to a 25-mL flask, and stirred at room temperature for 30 minutes. HATU (350 mg) and a compound 5 (211 mg, prepared by a reference: Wei B., et al. J Med Chem, 2018, 61, 989) were added sequentially, and reacted overnight at room temperature. The reaction solution was poured into 50 mL of ice water, extracted with ethyl acetate (20 mL×3), washed with 20 mL of a saturated saline solution, and dried over anhydrous sodium sulfate. A solvent was evaporated at a reduced pressure. The residue was separated by a column chromatography to obtain 420 mg of a white solid compound 6 (dichloromethane:methanol=100:5) with a yield of 82.0%. ¹H NMR (600 MHz, DMSO-d6) δ: 10.42 (s, 1H), 9.91 (s, 1H), 8.51 (t, J=5.8 Hz, 1H), 8.32 (d, J=8.4 Hz, 1H), 8.11 (d, J=7.8 Hz, 1H), 7.89 (d, J=8.7 Hz, 1H), 7.74 (t, J=6.9 Hz, 1H), 7.65-7.46 (m, 6H), 7.43-7.32 (m, 3H), 7.23 (d, J=8.5 Hz, 2H), 5.99 (s, 1H), 5.42 (s, 2H), 5.10 (t, J=5.7 Hz, 1H), 4.59 (d, J=5.9 Hz, 2H), 4.46-4.32 (m, 4H), 4.26-4.19 (m, 1H), 4.11 (q, J=5.3 Hz, 2H), 4.00-3.91 (m, 4H), 3.64-3.39 (m, 18H), 2.98 (ddd, J=46.1, 13.1, 6.5 Hz, 2H), 2.47 (t, J=6.8 Hz, 1H), 2.41-2.35 (m, 1H), 2.03-1.89 (m, 2H), 1.06 (t, J=7.0 Hz, 1H), 0.98 (s, 9H), 0.89-0.76 (m, 8H). ESI HRMS calcd C₆₀H₇₇C₁₂F₂N₉O₁₂ [M+H]⁺ m/z, 1124.5037; found 1124.5105.

The compound 6 (420 mg), 10 mL of THF, and 118 μL of pyridine were added to a 25-mL flask, and stirred for 15 minutes in an ice-water bath. Phenyl p-nitrochloroformate (137 mg) was then added and the mixture was slowly heated to room temperature to react overnight. A solvent was evaporated at a reduced pressure. The residue was separated by a column chromatography to obtain 250 mg of a white solid compound 7 (dichloromethane:methanol=100:10) with a yield of 52.5%. ESI HRMS calcd C₆₇H₈₀Cₗ₂F₂N₁₀O₁₆ [M+H]⁺ m/z, 1389.5099; found 1389.5178.

The compound 7 (50 mg), 2 mL of DMF, and the compound 8 (23 mg, prepared according to a method of example 7 in a patent application with a publication No. CN 110483608 A) were added to a 5-mL flask and stirred at room temperature for 30 minutes. HOBt (1 mg) and 29 μL of pyridine were sequentially added and the mixture was reacted overnight at room temperature. The reaction solution was poured into 50 mL of ice water, extracted with ethyl acetate (20 mL of ×3), washed with 20 mL of a saturated saline solution, and dried over anhydrous sodium sulfate, a solvent was evaporated at a reduced pressure, and the residue was separated by a column chromatography to obtain 28 mg of a white solid compound I-1 (dichloromethane:methanol=100:10) with a yield of 43.7%. ¹H NMR (600 MHz, DMSO) δ10.44 (s, 1H), 10.03 (s, 1H), 8.53 (s, 1H), 8.34 (d, J=8.4 Hz, 1H), 8.15 (d, J=7.2 Hz, 1H), 7.90 (d, J=8.6 Hz, 1H), 7.84 (d, J=9.6 Hz, 1H), 7.76 (t, J=6.8 Hz, 1H), 7.56 (ddd, J=38.3, 22.5, 8.3 Hz, 8H), 7.45-7.29 (m, 6H), 6.35 (d, J=9.8 Hz, 1H), 6.01 (s, 1H), 5.77 (s, 1H), 5.39 (d, J=63.3 Hz, 2H), 5.02 (d, J=16.9 Hz, 3H), 4.88 (s, 1H), 4.62 (s, 3H), 4.41 (s, 2H), 4.33-4.22 (m, 2H), 4.03-3.90 (m, 6H), 3.61 (d, J=6.2 Hz, 2H), 3.58-3.46 (m, 16H), 3.46-3.42 (m, 4H), 3.00 (dd, J=47.7, 6.4 Hz, 2H), 2.48 (d, J=7.3 Hz, 2H), 2.43-2.35 (m, 1H), 2.00 (dd, J=21.4, 9.2 Hz, 3H), 1.93-1.81 (m, 4H), 1.81-1.57 (m, 9H), 1.46 (s, 2H), 1.42-1.18 (m, 13H), 1.12 (s, 4H), 0.99 (s, 10H), 0.91-0.78 (m, 11H). ¹³C NMR (151 MHz, DMSO-d₆) δ213.74, 171.60, 171.50, 171.05, 170.78, 166.03, 161.62, 159.35, 154.91, 154.63, 150.69, 147.96, 147.64, 139.14, 131.90, 131.43, 130.47, 130.07, 129.75, 129.07, 128.97, 126.09, 125.75, 121.33, 120.52, 119.67, 119.49, 117.78, 117.49, 115.10, 110.07, 84.62, 73.62, 71.47, 70.23, 70.18, 70.15, 70.09, 69.93, 69.45, 67.37, 66.65, 65.09, 63.91, 63.71, 62.64, 57.96, 56.27, 55.36, 53.58, 50.62, 44.36, 43.66, 39.10, 36.97, 36.38, 33.12, 32.37, 31.05, 30.55, 29.96, 29.74, 29.48, 28.13, 27.28, 26.73, 25.93, 23.84, 21.74, 19.63, 18.56, 17.75. ESI HRMS calcd C₉₀H₁₁₅Cl₂F₂N₁₁O₂₀ [M+H]⁺ m/z, 1778.7655; found 1778.7738.

### EXAMPLE 2

### Preparation of Compound I-2

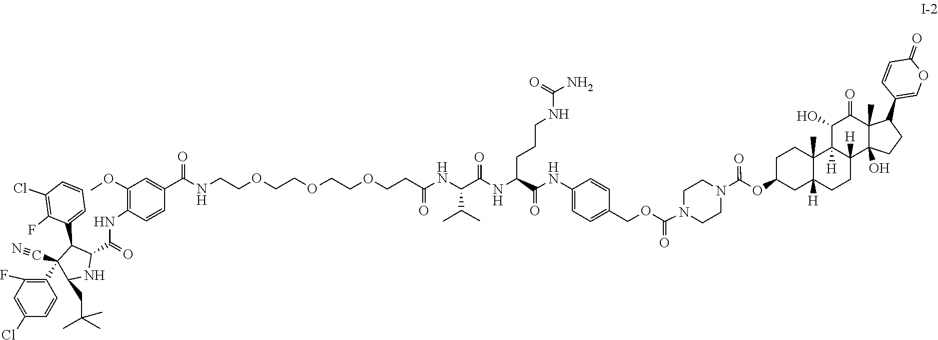

Following the method of example 1, a compound I-2 was obtained by substituting the compound 2 (1-amino-3,6,9,12-tetraoxapentadecan-15-oic acid) with 3-(2-(2-(2-aminoethoxy)ethoxy)ethoxy)propionic acid. ¹H NMR (600 MHz, DMSO) δ10.41 (s, 1H), 9.98 (s, 1H), 8.48 (t, J=5.6 Hz, 1H), 8.32 (d, J=8.5 Hz, 1H), 8.10 (d, J=7.4 Hz, 1H), 7.88-7.80 (m, 2H), 7.73 (t, J=6.9 Hz, 1H), 7.59 (dd, J=12.6, 9.3 Hz, 5H), 7.53 (t, J=7.6 Hz, 1H), 7.49 (d, J=8.4 Hz, 1H), 7.39 (t, J=8.5 Hz, 1H), 7.37-7.33 (m, 2H), 7.30 (d, J=8.6 Hz, 2H), 6.33 (d, J=9.7 Hz, 1H), 5.97 (s, 1H), 5.40 (s, 2H), 5.02 (s, 2H), 4.96 (s, 1H), 4.86 (s, 1H), 4.62-4.57 (m, 2H), 4.53 (d, J=4.8 Hz, 1H), 4.41-4.34 (m, 2H), 4.28 (dd, J=10.9, 4.8 Hz, 1H), 4.25-4.20 (m, 1H), 4.00-3.90 (m, 5H), 3.59 (dd, J=11.6, 6.1 Hz, 2H), 3.55-3.45 (m, 11H), 3.41 (dd, J=17.8, 12.1 Hz, 10H), 3.02 (dd, J=13.5, 6.6 Hz, 1H), 2.94 (dd, J=13.2, 6.7 Hz, 1H), 2.48-2.43 (m, 2H), 2.41-2.34 (m, 1H), 1.97 (dd, J=13.5, 6.7 Hz, 2H), 1.87 (dd, J=28.6, 14.7 Hz, 4H), 1.73 (t, J=11.2 Hz, 3H), 1.68-1.57 (m, 5H), 1.43 (d, J=10.4 Hz, 2H), 1.33 (d, J=17.6 Hz, 2H), 1.27-1.19 (m, 4H), 1.10 (s, 3H), 0.98 (s, 9H), 0.86 (d, J=6.8 Hz, 3H), 0.83 (d, J=6.8 Hz, 3H), 0.81 (s, 3H). HRMS (ESI, positive) m/z calcd for C₈₈H₁₁₁Cl₂F₂N₁₁O₁₉ [M+H]⁺: 1734.7481; found 1734.7400.

### EXAMPLE 3

### Preparation of Compound I-3

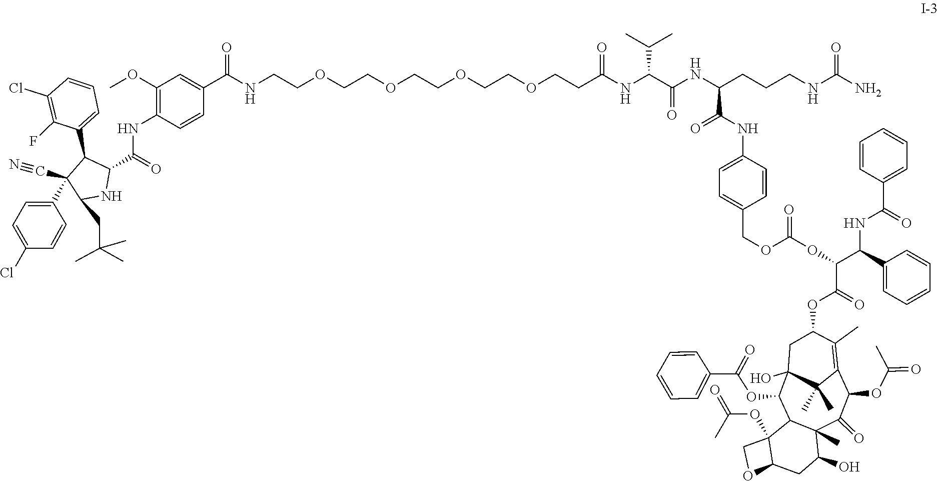

Following the method of example 1, 25 mg of a white solid compound I-3 was obtained by substituting the

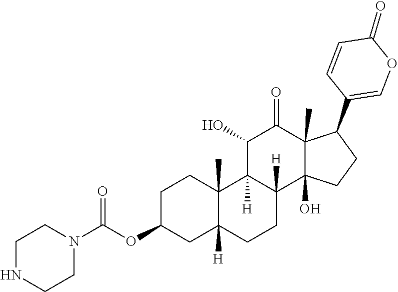

with paclitaxel. ¹H NMR (600 MHz, DMSO) δ10.41 (s, 1H), 10.04 (s, 1H), 9.27 (d, J=8.5 Hz, 1H), 8.50 (t, J=5.6 Hz, 1H), 8.32 (d, J=8.4 Hz, 1H), 8.14 (d, J=7.4 Hz, 1H), 8.02-7.96 (m, 2H), 7.87 (d, J=8.7 Hz, 1H), 7.84-7.80 (m, 2H), 7.73 (dd, J=13.3, 6.9 Hz, 2H), 7.64 (t, J=7.7 Hz, 2H), 7.62-7.56 (m, 4H), 7.56-7.52 (m, 2H), 7.51-7.42 (m, 7H), 7.39 (t, J=8.5 Hz, 1H), 7.37-7.33 (m, 2H), 7.31 (d, J=8.7 Hz, 2H), 7.19 (dd, J=7.4, 4.8 Hz, 1H), 6.31 (s, 1H), 5.98 (s, 1H), 5.84 (t, J=9.1 Hz, 1H), 5.54 (t, J=8.7 Hz, 1H), 5.42 (d, J=7.2 Hz, 3H), 5.36 (d, J=8.9 Hz, 1H), 5.14 (s, 2H), 4.92 (t, J=8.9 Hz, 2H), 4.65 (s, 1H), 4.59 (d, J=5.9 Hz, 2H), 4.39 (d, J=5.7 Hz, 2H), 4.26-4.22 (m, 1H), 4.13 (dd, J=17.8, 7.0 Hz, 1H), 4.02 (dd, J=16.9, 8.3 Hz, 2H), 3.98-3.90 (m, 4H), 3.63-3.56 (m, 3H), 3.48 (dddd, J=21.1, 16.4, 9.0, 3.9 Hz, 17H), 2.99 (ddd, J=19.4, 13.3, 6.5 Hz, 2H), 2.47 (t, J=7.0 Hz, 1H), 2.41-2.30 (m, 2H), 2.26 (s, 3H), 2.12 (s, 3H), 1.97 (dq, J=13.3, 6.7 Hz, 1H), 1.82 (s, 3H), 1.75-1.57 (m, 4H), 1.56-1.49 (m, 4H), 1.45 (s, 1H), 1.36 (d, J=5.0 Hz, 1H), 1.27 (d, J=13.5 Hz, 1H), 1.04 (s, 3H), 1.01 (s, 3H), 0.97 (s, 9H), 0.86 (d, J=6.7 Hz, 3H), 0.83 (d, J=6.8 Hz, 3H). ¹³C NMR (151 MHz, DMSO-d₆) δ171.63, 171.49, 170.76, 170.15, 169.41, 169.23, 166.84, 166.02, 159.35, 139.64, 134.53, 133.96, 132.01, 131.44, 130.47, 130.41, 130.04, 129.76, 129.21, 129.14, 128.79, 128.05, 127.86, 126.09, 125.74, 120.51, 119.99, 119.49, 117.77, 117.49, 110.08, 84.09, 80.75, 77.62, 77.16, 75.76, 75.19, 74.96, 70.87, 70.23, 70.18, 70.15, 70.09, 69.92, 69.44, 67.37, 65.08, 63.67, 57.94, 57.87, 56.27, 54.40, 53.62, 50.62, 46.56, 44.36, 43.42, 36.99, 36.38, 34.84, 31.04, 30.54, 29.96, 29.69, 27.29, 26.80, 22.99, 21.82, 21.13, 19.63, 18.56, 14.38, 10.23. ESI HRMS calcd C₁₀₈H₁₂₉Cl₂F₂N₁₀O₂₇ [M+H]⁺ m/z, 2103.8139; found 2103.8212.

### EXAMPLE 4

### Preparation of Compound I-4

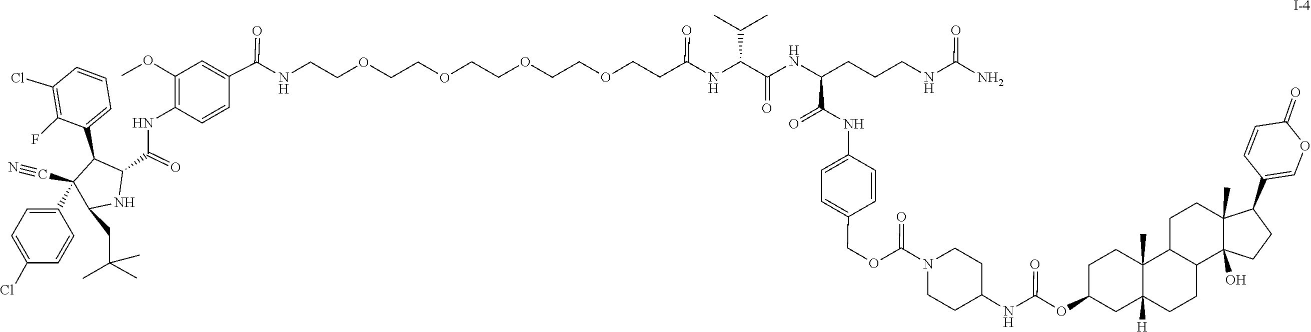

Following the method of example 1, 30 mg of a pale green solid compound I-4 was obtained by substituting the

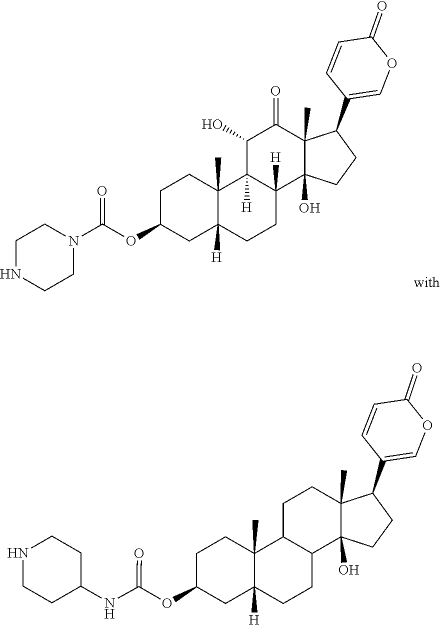

(prepared according to a method of example 15 in a patent application with a publication No. CN102532235A). ¹H NMR (600 MHz, DMSO) δ10.41 (s, 1H), 9.98 (s, 1H), 8.50 (t, J=5.6 Hz, 1H), 8.32 (d, J=8.4 Hz, 1H), 8.11 (d, J=7.5 Hz, 1H), 7.94 (dd, J=9.7, 2.5 Hz, 1H), 7.87 (d, J=8.7 Hz, 1H), 7.73 (t, J=6.9 Hz, 1H), 7.58 (dd, J=11.2, 5.1 Hz, 4H), 7.55-7.51 (m, 2H), 7.50-7.47 (m, 1H), 7.39 (t, J=8.4 Hz, 1H), 7.37-7.33 (m, 2H), 7.27 (dd, J=14.6, 8.4 Hz, 3H), 6.29 (d, J=9.9 Hz, 1H), 5.97 (s, 1H), 5.41 (s, 2H), 4.94 (s, 2H), 4.84 (s, 1H), 4.59 (d, J=6.1 Hz, 2H), 4.42-4.34 (m, 2H), 4.25-4.20 (m, 1H), 4.16 (s, 1H), 3.98-3.91 (m, 4H), 3.88 (d, J=13.3 Hz, 2H), 3.58 (dt, J=9.4, 4.8 Hz, 2H), 3.55-3.45 (m, 14H), 3.41 (dd, J=11.7, 5.9 Hz, 2H), 3.06-2.90 (m, 3H), 2.47 (d, J=7.2 Hz, 2H), 2.38 (dd, J=13.4, 7.1 Hz, 1H), 2.11-1.88 (m, 4H), 1.83-1.67 (m, 5H), 1.61 (dt, J=17.2, 11.8 Hz, 7H), 1.54-1.29 (m, 10H), 1.29-1.15 (m, 6H), 1.06 (s, 2H), 0.98 (s, 9H), 0.89 (s, 3H), 0.86 (d, J=6.8 Hz, 3H), 0.82 (d, J=6.8 Hz, 3H), 0.60 (s, 3H). ¹³C NMR (151 MHz, DMSO-d₆) δ171.03, 170.92, 170.45, 170.19, 165.43, 158.76, 155.22, 153.91, 149.12, 147.39, 147.32, 130.87, 129.91, 129.50, 128.58, 125.52, 125.19, 122.65, 119.95, 118.81, 116.92, 114.09, 109.51, 83.31, 70.41, 69.66, 69.62, 69.58, 69.52, 69.36, 68.89, 66.81, 64.88, 64.52, 63.15, 57.39, 55.70, 52.99, 50.05 48.49, 47.95, 47.41, 43.79, 42.12, 41.11, 36.87, 35.82, 34.76, 31.91, 30.46, 30.17, 29.98, 29.40, 29.17, 28.38, 26.73, 26.23, 24.63, 23.69, 21.01, 20.92, 19.07, 17.99, 16.56. ESI HRMS calcd C₉₁H₁₁₉Cl₂F₂N₁₁O₁₈ [M+H]⁺ m/z, 1762.8080; found 1762.8152.

### EXAMPLE 5

### Preparation of Compound I-5

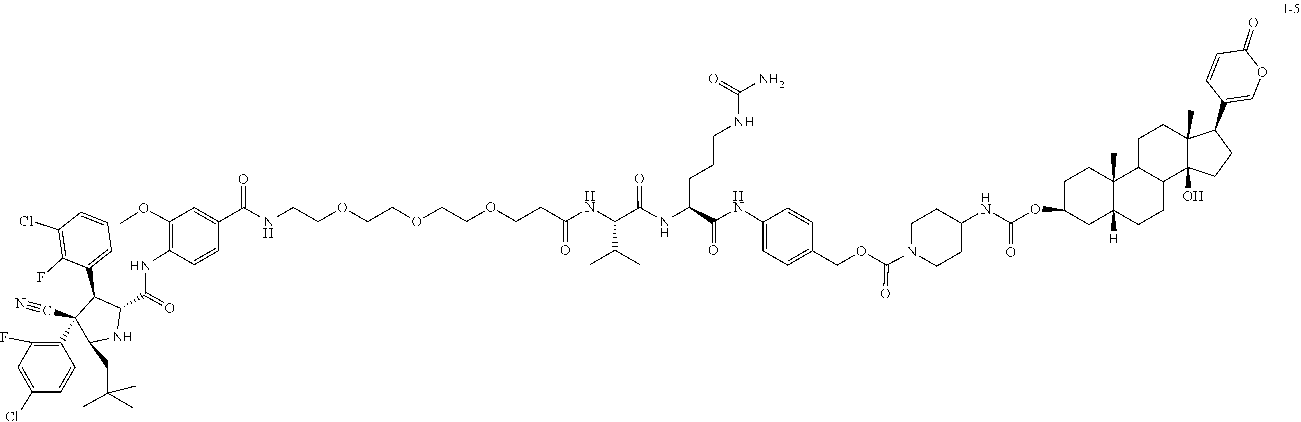 [SMILES: COc1cc(C(=O)NCCOCCOCCOCCC(=O)N[C@H](C(=O)N[C@@H](CCCNC(N)=O)C(=O)Nc2ccc(COC(=O)N3CCC(NC(=O)O[C@H]4CC[C@]5(C)C6CC[C@]7(C)[C@@H](c8ccc(=O)oc8)CC[C@]7(O)C6CC[C@@H]5C4)CC3)cc2)C(C)C)ccc1NC(=O)[C@@H]1N[C@@H](CC(C)(C)C)[C@](C#N)(c2ccc(Cl)cc2F)[C@H]1c1cccc(Cl)c1F]

Following the method of example 1, a compound I-5 was obtained by respectively substituting the

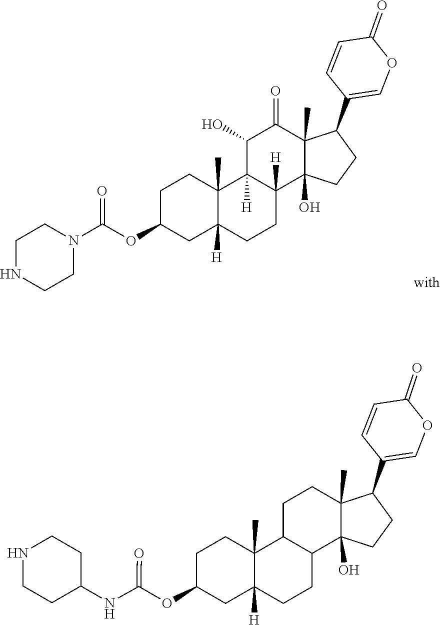

and substituting the compound 2 (1-amino-3,6,9,12-tetraoxapentadecan-15-oic acid) with 3-(2-(2-(2-aminoethoxy)ethoxy)ethoxy)propionic acid. ¹H NMR (600 MHz, DMSO) δ10.40 (s, 1H), 9.96 (s, 1H), 8.48 (s, 1H), 8.31 (d, J=8.3 Hz, 1H), 8.08 (d, J=7.8 Hz, 1H), 7.95-7.91 (m, 1H), 7.85 (d, J=8.6 Hz, 1H), 7.72 (d, J=7.2 Hz, 1H), 7.60-7.56 (m, 4H), 7.55-7.51 (m, 2H), 7.48 (d, J=6.6 Hz, 1H), 7.39 (t, J=8.5 Hz, 1H), 7.36-7.33 (m, 2H), 7.28 (d, J=8.8 Hz, 2H), 7.23 (s, 1H), 6.28 (d, J=9.3 Hz, 1H), 5.96 (s, 1H), 5.40 (s, 2H), 4.93 (s, 2H), 4.84 (s, 1H), 4.59 (d, J=5.9 Hz, 2H), 4.38 (s, 2H), 4.24-4.20 (m, 1H), 4.14 (s, 1H), 3.98-3.91 (m, 4H), 3.86 (s, 2H), 3.61-3.56 (m, 2H), 3.49 (tt, J=11.7, 6.2 Hz, 11H), 3.41 (d, J=5.9 Hz, 2H), 3.01 (s, 1H), 2.94 (s, 1H), 2.45 (d, J=6.7 Hz, 2H), 2.37 (d, J=14.8 Hz, 2H), 2.07 (s, 2H), 1.95 (dd, J=18.6, 12.2 Hz, 2H), 1.78 (s, 5H), 1.61 (d, J=27.6 Hz, 7H), 1.49 (s, 4H), 1.36 (d, J=29.9 Hz, 6H), 1.23 (t, J=15.4 Hz, 7H), 0.97 (s, 9H), 0.89 (d, J=9.2 Hz, 3H), 0.85 (d, J=6.7 Hz, 3H), 0.82 (d, J=6.8 Hz, 3H), 0.59 (s, 3H). HRMS (ESI, positive) m/z calcd for C₈₉H₁₁₅Cl₂F₂N₁₁O₁₇ [M+H]⁺: 1718.7896; found 1718.7890.

### EXAMPLE 6

### Preparation of Compound I-6

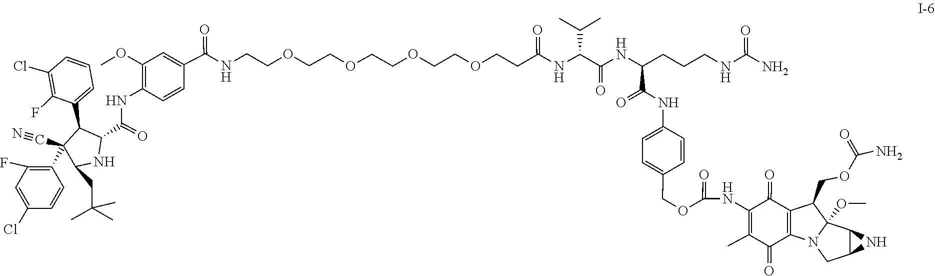

Following the method of example 1, 25 mg of a purple solid compound I-6 was obtained by substituting

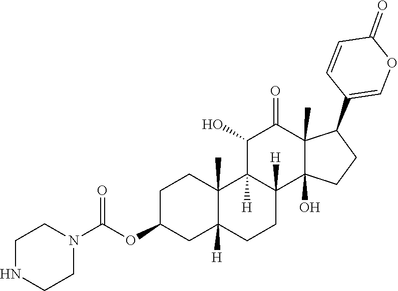

with mitomycin C. ¹H NMR (600 MHz, DMSO) δ10.41 (s, 1H), 9.99 (s, 1H), 8.50 (t, J=5.5 Hz, 1H), 8.32 (d, J=8.4 Hz, 1H), 8.12 (d, J=7.4 Hz, 1H), 7.87 (d, J=8.7 Hz, 1H), 7.74 (t, J=6.8 Hz, 1H), 7.63-7.52 (m, 5H), 7.49 (dd, J=8.4, 1.5 Hz, 1H), 7.40 (t, J=8.5 Hz, 1H), 7.37-7.34 (m, 2H), 7.31 (d, J=8.6 Hz, 2H), 7.06 (s, 1H), 6.64 (d, J=95.7 Hz, 2H), 5.97 (s, 1H), 5.41 (s, 2H), 4.96 (dd, J=34.0, 12.4 Hz, 2H), 4.72 (dd, J=10.5, 4.5 Hz, 1H), 4.59 (d, J=5.7 Hz, 2H), 4.41-4.35 (m, 2H), 4.23 (dd, J=7.7, 6.0 Hz, 2H), 3.99-3.89 (m, 5H), 3.63-3.56 (m, 3H), 3.55-3.37 (m, 20H), 3.13 (s, 3H), 3.05-2.91 (m, 2H), 2.47 (dd, J=14.3, 7.1 Hz, 1H), 2.37 (dd, J=13.6, 7.2 Hz, 1H), 1.96 (dd, J=13.5, 6.7 Hz, 1H), 1.78-1.62 (m, 5H), 1.58 (d, J=9.4 Hz, 1H), 1.39 (d, J=48.5 Hz, 2H), 1.27 (d, J=13.5 Hz, 1H), 0.98 (s, 9H), 0.84 (dd, J=20.0, 6.7 Hz, 6H). ¹³C NMR (151 MHz, DMSO-d₆) δ177.09, 176.26, 171.58, 171.50, 171.04, 170.73, 166.01, 159.33, 159.14, 157.03, 154.94, 149.25, 147.96, 130.47, 130.06, 129.74, 129.45, 129.06, 126.44, 126.08, 125.74, 120.51, 119.39, 118.12, 117.93, 117.77, 117.48, 110.07, 110.02, 105.78, 103.36, 70.22, 70.17, 70.14 70.08, 69.91, 69.44, 67.93, 67.37, 65.08, 63.86, 63.71, 61.00, 57.91, 56.27, 53.56, 50.63 49.75, 48.98, 44.35, 43.39, 42.28, 36.38, 31.05, 30.54, 29.96, 29.75, 27.25, 19.63, 18.55, 8.79. ESI HRMS calcd C₇₆H₉₃Cl₂F₂N₁₃O₁₈ [M+H]⁺ m/z, 1584.6107; found 1584.6179.

### EXAMPLE 7

### Preparation of Compound I-7

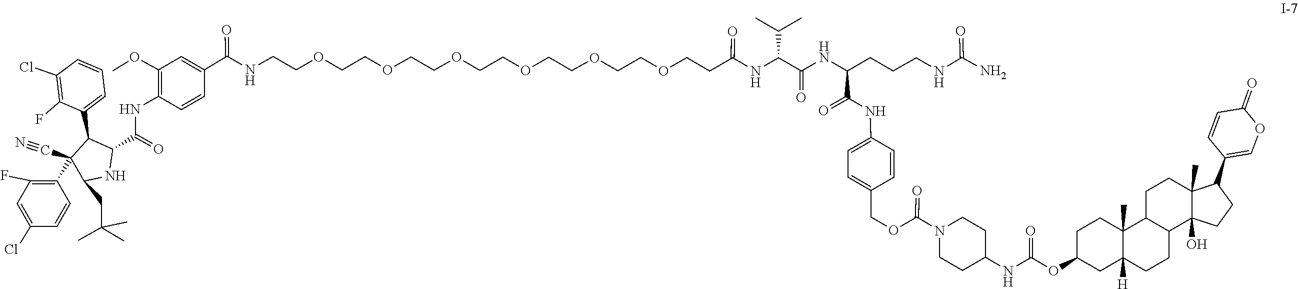

Following the method of example 1, a compound I-7 was obtained by respectively substituting the

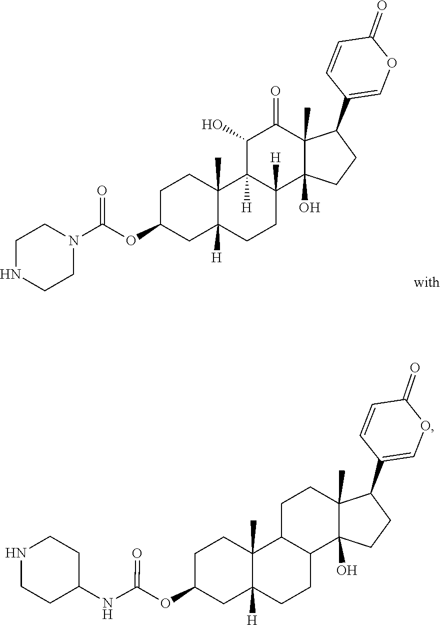

and substituting the compound 2 (1-amino-3,6,9,12-tetraoxapentadecan-15-oic acid) with 1-amino-3,6,9,12,15,18-hexaoxahenicosan-21-oic acid. ¹H NMR (600 MHz, DMSO) δ10.41 (s, 1H), 9.97 (s, 1H), 8.48 (t, J=5.8 Hz, 1H), 8.32 (d, J=8.4 Hz, 1H), 8.10 (d, J=7.3 Hz, 1H), 7.93 (dd, J=9.7, 2.4 Hz, 1H), 7.86 (d, J=8.5 Hz, 1H), 7.73 (t, J=6.6 Hz, 1H), 7.58 (dd, J=11.3, 5.1 Hz, 4H), 7.56-7.51 (m, 2H), 7.49 (dd, J=8.5, 1.7 Hz, 1H), 7.39 (t, J=8.5 Hz, 1H), 7.37-7.33 (m, 2H), 7.26 (dd, J=18.5, 8.2 Hz, 3H), 6.29 (d, J=9.7 Hz, 1H), 5.97 (s, 1H), 5.40 (s, 2H), 4.94 (s, 2H), 4.84 (s, 1H), 4.61-4.57 (m, 2H), 4.37 (s, 2H), 4.25-4.19 (m, 1H), 4.15 (s, 1H), 3.99-3.91 (m, 4H), 3.88 (d, J=12.6 Hz, 2H), 3.62-3.57 (m, 2H), 3.55-3.46 (m, 22H), 3.44-3.39 (m, 2H), 3.02 (dd, J=13.6, 6.7 Hz, 1H), 2.94 (dd, J=13.0, 6.3 Hz, 1H), 2.48-2.44 (m, 2H), 2.38 (t, J=6.3 Hz, 1H), 2.10-2.01 (m, 2H), 1.99-1.89 (m, 2H), 1.75 (d, J=32.6 Hz, 5H), 1.65-1.55 (m, 6H), 1.50 (t, J=18.9 Hz, 4H), 1.36 (dd, J=43.6, 13.5 Hz, 6H), 1.29-1.14 (m, 8H), 1.08 (s, 2H), 0.98 (s, 9H), 0.89 (s, 3H), 0.86 (d, J=6.8 Hz, 3H), 0.82 (d, J=6.7 Hz, 3H), 0.60 (s, 3H). HRMS (ESI, positive) m/z calcd for C₉₅H₁₂₇Cl₂F₂N₁₁O₂₀ [M+H]⁺: 1850.8682; found 1850.8677.

### EXAMPLE 8

### Preparation of Compound I-8

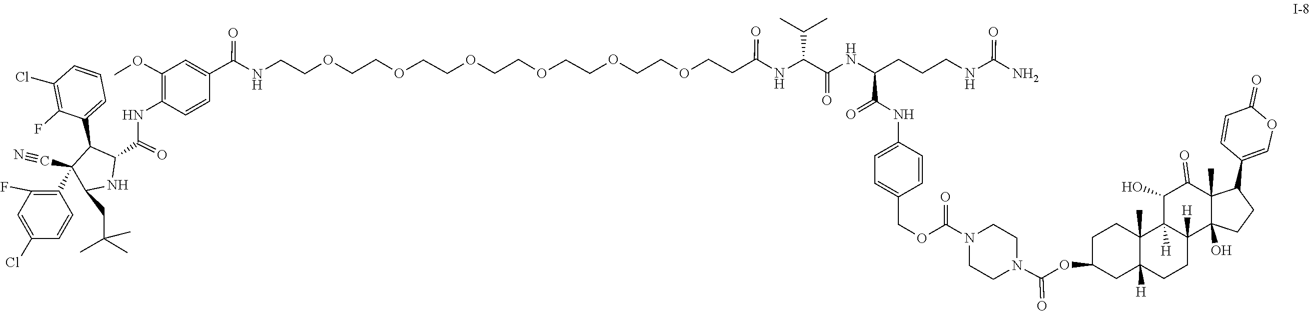

Following the method of example 1, a compound I-8 was prepared by substituting the compound 2 (1-amino-3,6,9,12-tetraoxapentadecan-15-oic acid) with 1-amino-3,6,9,12,15,18-hexaoxahenicosan-21-oic acid. ¹H NMR (600 MHz, DMSO) δ10.40 (s, 1H), 9.98 (s, 1H), 8.47 (t, J=5.6 Hz, 1H), 8.32 (d, J=8.4 Hz, 1H), 8.10 (d, J=7.5 Hz, 1H), 7.87-7.80 (m, 2H), 7.73 (t, J=6.8 Hz, 1H), 7.61-7.56 (m, 5H), 7.53 (t, J=7.0 Hz, 1H), 7.51-7.47 (m, 1H), 7.39 (t, J=8.4 Hz, 1H), 7.37-7.33 (m, 2H), 7.31 (d, J=8.6 Hz, 2H), 6.33 (d, J=9.7 Hz, 1H), 5.96 (d, J=5.8 Hz, 1H), 5.40 (s, 2H), 5.02 (s, 2H), 4.96 (s, 1H), 4.86 (s, 1H), 4.61-4.57 (m, 2H), 4.53 (d, J=4.8 Hz, 1H), 4.39 (dd, J=13.5, 8.0 Hz, 2H), 4.28 (dd, J=11.1, 4.9 Hz, 1H), 4.23 (dd, J=8.5, 6.8 Hz, 1H), 4.00-3.90 (m, 5H), 3.63-3.57 (m, 2H), 3.56-3.46 (m, 23H), 3.44-3.36 (m, 8H), 3.02 (dd, J=13.3, 6.6 Hz, 1H), 2.94 (dd, J=12.9, 6.4 Hz, 1H), 2.47 (t, J=7.0 Hz, 2H), 2.41-2.35 (m, 1H), 2.03-1.94 (m, 2H), 1.92-1.82 (m, 4H), 1.73 (t, J=11.5 Hz, 3H), 1.63 (ddd, J=24.4, 14.0, 9.7 Hz, 5H), 1.43 (d, J=9.8 Hz, 2H), 1.35 (dd, J=24.4, 11.4 Hz, 2H), 1.30-1.19 (m, 5H), 1.10 (s, 3H), 0.98 (s, 9H), 0.86 (d, J=6.8 Hz, 3H), 0.83 (d, J=6.8 Hz, 3H), 0.81 (s, 3H). HRMS (ESI, positive) m/z calcd for C₉₄H₁₂₃Cl₂F₂N11O₂₂ [M+H]⁺: 1866.8268; found 1186.8262.

### EXAMPLE 9

### Preparation of Compound I-9

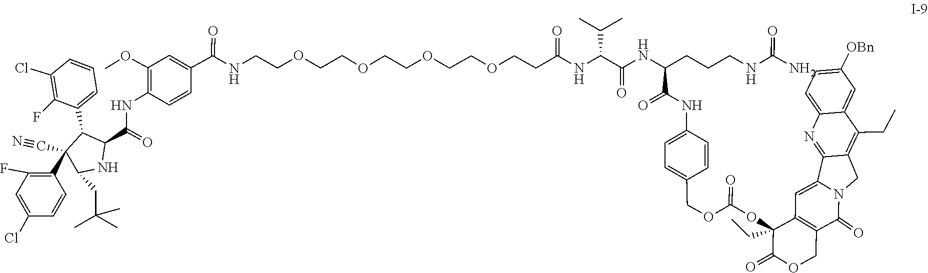 [SMILES: CCc1c2c(nc3ccc(OCc4ccccc4)cc13)-c1cc3c(c(=O)n1C2)COC(=O)[C@@]3(CC)OC(=O)OCc1ccc(NC(=O)[C@H](CCCNC(N)=O)NC(=O)[C@H](NC(=O)CCOCCOCCOCCOCCNC(=O)c2ccc(NC(=O)[C@H]3N[C@H](CC(C)(C)C)[C@@](C#N)(c4ccc(Cl)cc4F)[C@@H]3c3cccc(Cl)c3F)c(OC)c2)C(C)C)cc1]

Following the method of example 1, a compound I-9 was obtained by substituting the

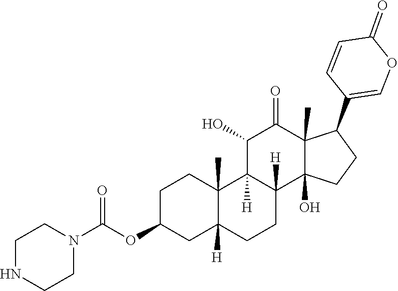

with 10-benzyl-7-ethylcamptothecin. ¹H NMR (300 MHz, DMSO) δ10.41 (s, 1H), 10.03 (s, 1H), 8.50 (s, 1H), 8.31 (d, J=8.5 Hz, 1H), 8.12 (d, J=9.4 Hz, 2H), 7.88 (d, J=8.9 Hz, 1H), 7.73 (s, 1H), 7.58 (d, J=10.3 Hz, 9H), 7.48 (d, J=8.6 Hz, 2H), 7.45-7.27 (m, 10H), 7.18 (s, 1H), 6.97 (s, 1H), 6.00 (s, 1H), 5.51 (s, 2H), 5.44-5.30 (m, 6H), 5.09 (d, J=5.5 Hz, 2H), 4.60 (s, 2H), 4.37 (s, 2H), 4.24 (s, 1H), 3.99-3.89 (m, 4H), 3.51 (dd, J=25.2, 12.1 Hz, 18H), 3.18 (d, J=6.6 Hz, 2H), 2.98 (d, J=8.1 Hz, 2H), 2.17 (d, J=7.0 Hz, 2H), 2.00 (d, J=7.3 Hz, 4H), 1.64 (s, 3H), 1.30 (s, 7H), 0.97 (s, 9H), 0.86 (s, 3H). ESI HRMS calcd C₉₀H₁₀₁Cl₂F₂N₁₁O₁₈ [M+Na]⁺ m/z, 1754.6569; found 1754.6572.

### EXAMPLE 10

### Preparation of Compound I-10

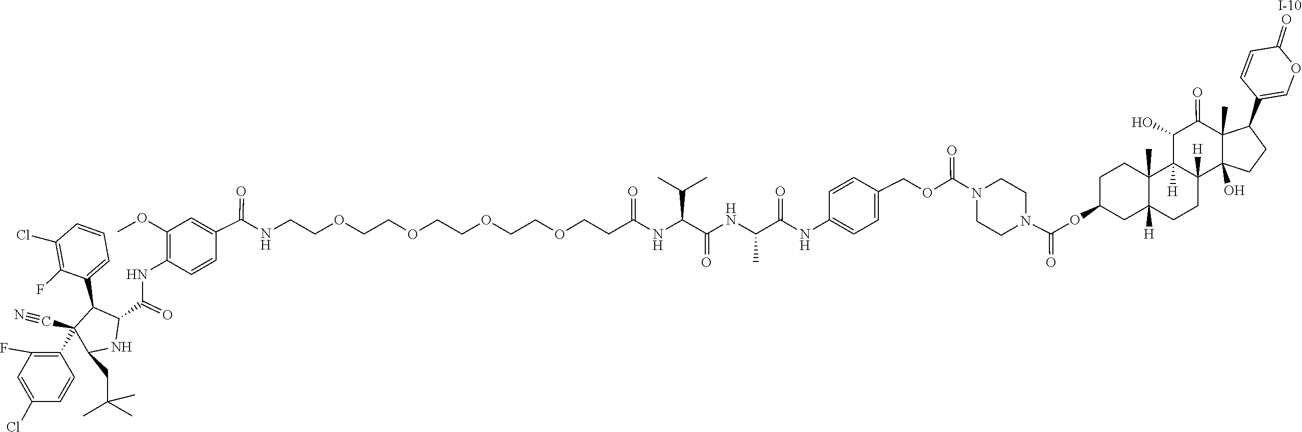

Following the method of example 1, a compound I-10 was obtained by substituting the Val-Cit with Val-Ala. ¹H NMR (600 MHz, DMSO) δ10.40 (s, 1H), 9.99 (s, 1H), 8.48 (s, 1H), 8.37 (d, J=6.6 Hz, 1H), 8.31 (d, J=8.3 Hz, 1H), 7.93 (d, J=9.5 Hz, 1H), 7.76-7.66 (m, 8H), 7.57 (d, J=8.0 Hz, 4H), 7.55-7.50 (m, 2H), 7.46 (dd, J=23.9, 8.7 Hz, 2H), 7.42-7.32 (m, 3H), 7.26 (dd, J=21.8, 7.9 Hz, 3H), 6.28 (d, J=9.7 Hz, 1H), 5.59 (s, 1H), 4.95 (d, J=22.1 Hz, 2H), 4.84 (s, 1H), 4.62-4.57 (m, 2H), 4.42-4.35 (m, 2H), 4.30-4.27 (m, 1H), 4.17-4.10 (m, 8H), 4.04 (t, J=6.5 Hz, 2H), 4.01-3.90 (m, 10H), 3.87 (d, J=13.2 Hz, 2H), 3.63-3.48 (m, 12H), 3.40 (d, J=5.9 Hz, 3H), 2.89-2.84 (m, 3H), 2.72 (d, J=15.1 Hz, 2H), 2.45 (s, 1H), 1.62 (dd, J=15.4, 9.3 Hz, 7H), 1.53 (ddd, J=20.4, 14.2, 6.2 Hz, 10H), 1.33-1.25 (m, 38H), 0.97 (s, 9H), 0.59 (s, 3H).

### EXAMPLE 11

### Preparation of Compound I-11

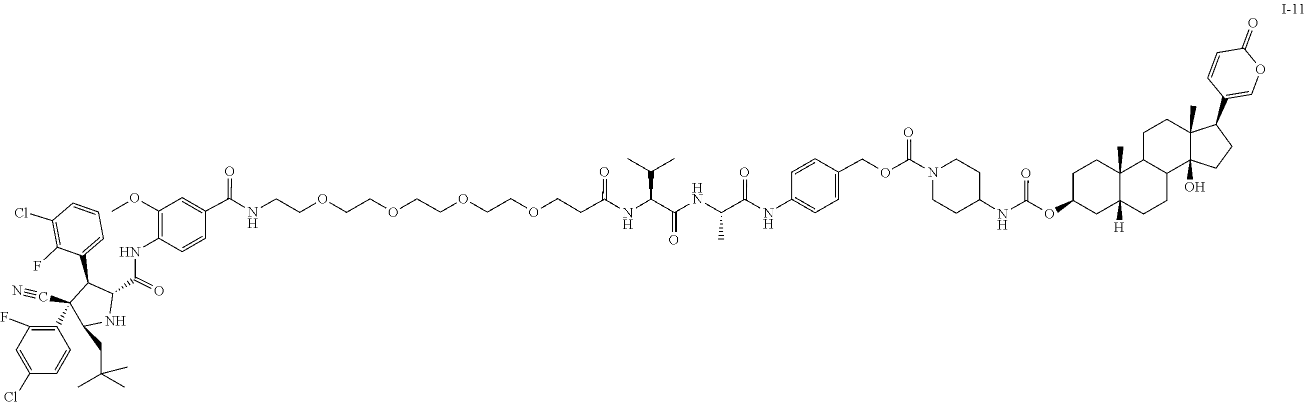 [SMILES: COc1cc(C(=O)NCCOCCOCCOCCOCCC(=O)N[C@H](C(=O)N[C@@H](C)C(=O)Nc2ccc(COC(=O)N3CCC(NC(=O)O[C@H]4CC[C@]5(C)C6CC[C@]7(C)[C@@H](c8ccc(=O)oc8)CC[C@]7(O)C6CC[C@@H]5C4)CC3)cc2)C(C)C)ccc1NC(=O)[C@@H]1N[C@@H](CC(C)(C)C)[C@](C#N)(c2ccc(Cl)cc2F)[C@H]1c1cccc(Cl)c1F]

Following the method of example 1, a compound I-11 was obtained by respectively substituting the

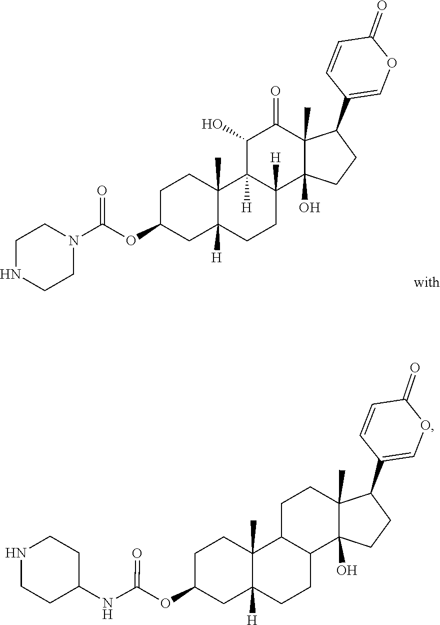

and the Val-Cit with Val-Ala. ¹H NMR (600 MHz, DMSO) δ10.40 (s, 1H), 10.09 (s, 1H), 8.52 (t, J=5.6 Hz, 1H), 8.40 (d, J=7.0 Hz, 1H), 8.31 (d, J=8.4 Hz, 1H), 7.81 (dd, J=9.8, 2.3 Hz, 1H), 7.73 (t, J=6.8 Hz, 1H), 7.58 (dd, J=14.1, 11.2 Hz, 5H), 7.53 (t, J=7.6 Hz, 1H), 7.47 (dd, J=18.0, 8.8 Hz, 2H), 7.39 (t, J=8.5 Hz, 1H), 7.36-7.33 (m, 2H), 7.29 (d, J=8.5 Hz, 2H), 6.32 (d, J=9.7 Hz, 1H), 5.00 (d, J=10.3 Hz, 3H), 4.85 (s, 1H), 4.59 (s, 2H), 4.53 (d, J=4.7 Hz, 1H), 4.40 (dd, J=14.3, 7.2 Hz, 2H), 4.28 (dt, J=11.9, 5.9 Hz, 2H), 3.99-3.89 (m, 8H), 3.61-3.49 (m, 11H), 3.40 (dd, J=15.7, 10.0 Hz, 9H), 2.45 (d, J=12.8 Hz, 1H), 2.00 (dd, J=13.4, 6.9 Hz, 2H), 1.86 (dd, J=26.6, 15.9 Hz, 4H), 1.72 (t, J=11.3 Hz, 2H), 1.68-1.59 (m, 4H), 1.52 (dd, J=14.6, 7.4 Hz, 1H), 1.43 (d, J=13.4 Hz, 1H), 1.33-1.27 (m, 7H), 1.22 (dd, J=19.4, 13.7 Hz, 7H), 1.09 (s, 3H), 0.97 (s, 9H), 0.87 (dd, J=8.0, 5.1 Hz, 6H), 0.83-0.79 (m, 6H).

### EXAMPLE 12

### Preparation of Compound I-12

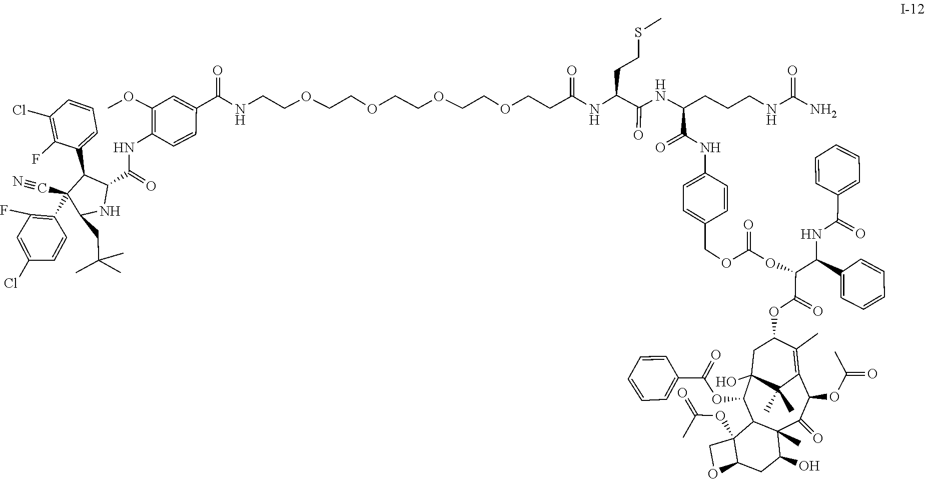

Following the method of example 1, a compound I-12 was obtained by respectively substituting the Val-Cit with Met-Cit, and the

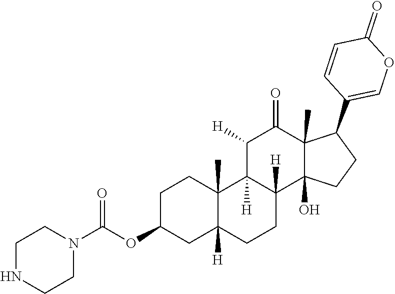

with paclitaxel. ¹H NMR (600 MHz, DMSO) δ10.40 (s, 1H), 10.08 (s, 1H), 9.28 (d, J=8.7 Hz, 1H), 8.50 (s, 1H), 8.32 (d, J=8.4 Hz, 1H), 8.14 (d, J=7.7 Hz, 1H), 8.08 (d, J=7.9 Hz, 1H), 7.99 (d, J=7.2 Hz, 2H), 7.82 (d, J=7.5 Hz, 2H), 7.73 (dd, J=13.4, 6.9 Hz, 2H), 7.63 (dd, J=17.3, 8.3 Hz, 4H), 7.59-7.53 (m, 4H), 7.50-7.43 (m, 7H), 7.39 (t, J=8.6 Hz, 1H), 7.35 (dd, J=7.3, 3.6 Hz, 2H), 7.31 (d, J=8.5 Hz, 2H), 7.20 (d, J=4.1 Hz, 1H), 6.31 (s, 1H), 6.02 (s, 1H), 5.84 (s, 1H), 5.54 (t, J=8.6 Hz, 1H), 5.42 (s, 3H), 5.37 (d, J=8.8 Hz, 1H), 5.14 (s, 2H), 4.92 (d, J=7.7 Hz, 2H), 4.64 (s, 1H), 4.60 (s, 2H), 4.42-4.36 (m, 3H), 4.12 (s, 1H), 4.02 (dd, J=16.9, 8.3 Hz, 2H), 3.96-3.90 (m, 4H), 3.59 (dd, J=10.1, 5.4 Hz, 3H), 3.49 (ddd, J=16.8, 7.7, 4.7 Hz, 16H), 3.41 (d, J=5.7 Hz, 2H), 3.05-3.00 (m, 1H), 2.98-2.94 (m, 1H), 2.46-2.44 (m, 2H), 2.38-2.32 (m, 2H), 2.26 (s, 3H), 2.12 (s, 3H), 2.02 (s, 3H), 1.88 (s, 2H), 1.82 (s, 3H), 1.51 (s, 3H), 1.26-1.23 (m, 6H), 1.02 (d, J=16.1 Hz, 6H), 0.97 (s, 9H). ESI HRMS calcd C₁₀₈H₁₂₆Cl₂F₂N₁₁O₂₇S [M+H]⁺ m/z, 2135.7938; found 2135.7932

### EXAMPLE 13

### Preparation of Compound I-13

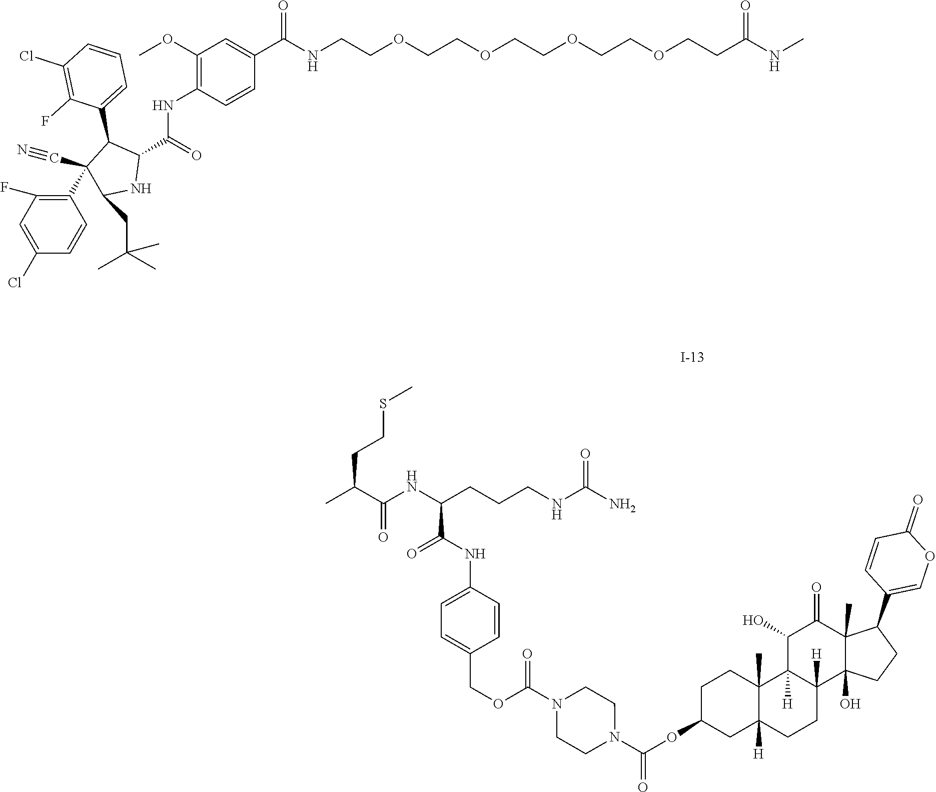

Following the method of example 1, a compound I-13 was obtained by substituting the Val-Cit with Met-Cit. ¹H NMR (600 MHz, DMSO) δ10.40 (s, 1H), 10.01 (s, 1H), 8.48 (s, 1H), 8.32 (d, J=8.4 Hz, 1H), 8.09 (dd, J=26.9, 7.8 Hz, 2H), 7.82 (d, J=9.5 Hz, 1H), 7.73 (t, J=6.6 Hz, 1H), 7.59 (dd, J=16.5, 9.0 Hz, 5H), 7.53 (t, J=7.3 Hz, 1H), 7.49 (d, J=8.3 Hz, 1H), 7.39 (t, J=8.3 Hz, 1H), 7.35 (d, J=5.7 Hz, 2H), 7.31 (d, J=8.5 Hz, 2H), 6.33 (d, J=10.0 Hz, 1H), 5.98 (s, 1H), 5.41 (s, 2H), 5.02 (s, 2H), 4.96 (s, 1H), 4.86 (s, 1H), 4.59 (d, J=6.0 Hz, 2H), 4.53 (d, J=4.6 Hz, 1H), 4.39 (s, 3H), 4.31-4.25 (m, 1H), 4.00-3.90 (m, 5H), 3.59 (dd, J=14.3, 6.8 Hz, 2H), 3.56-3.45 (m, 15H), 3.44-3.35 (m, 9H), 3.06-2.92 (m, 2H), 2.44 (dd, J=15.7, 9.0 Hz, 4H), 2.36 (dd, J=13.8, 7.1 Hz, 1H), 2.02 (s, 3H), 1.86 (d, J=32.6 Hz, 5H), 1.79-1.69 (m, 4H), 1.63 (dd, J=16.1, 9.8 Hz, 4H), 1.43 (d, J=13.5 Hz, 2H), 1.32-1.20 (m, 9H), 1.10 (s, 3H), 0.98 (s, 9H), 0.80 (s, 3H).

### EXAMPLE 14

### Preparation of Compound I-14

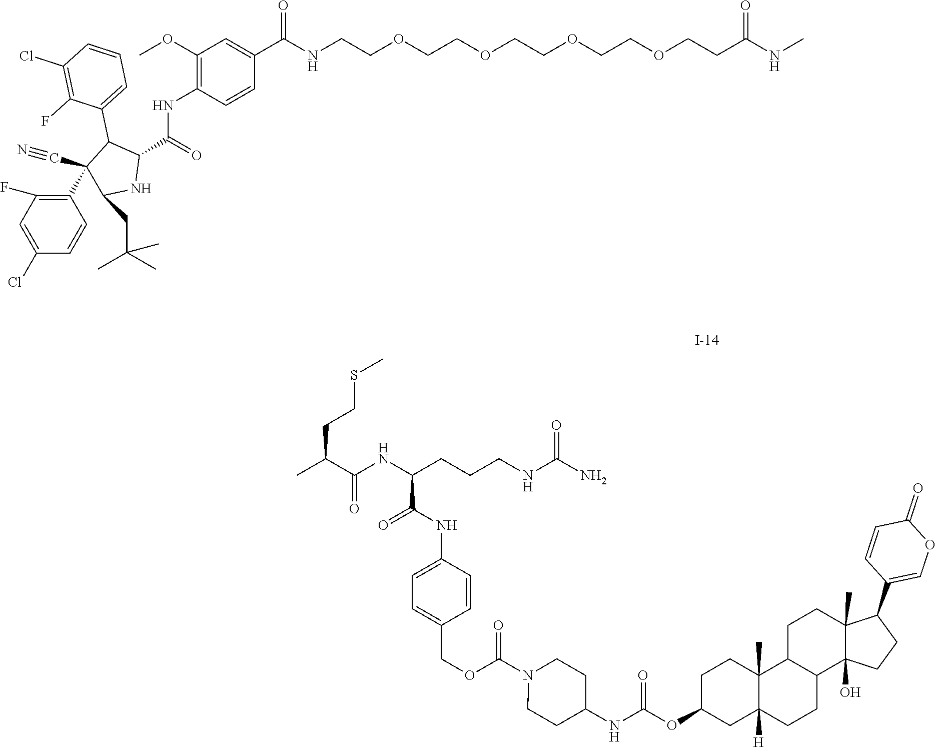

Following the method of example 1, a compound I-14 was obtained by respectively substituting the

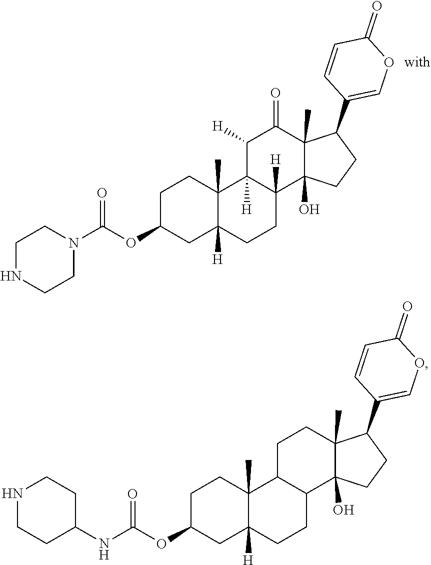

and the Val-Cit with Met-Cit. ¹H NMR (600 MHz, DMSO) δ10.41 (s, 1H), 10.00 (s, 1H), 8.49 (t, J=5.7 Hz, 1H), 8.32 (d, J=8.4 Hz, 1H), 8.08 (dd, J=21.1, 7.7 Hz, 2H), 7.93 (dd, J=9.7, 2.5 Hz, 1H), 7.73 (t, J=7.2 Hz, 1H), 7.58 (dd, J=15.0, 5.2 Hz, 4H), 7.55-7.51 (m, 2H), 7.50-7.47 (m, 1H), 7.39 (t, J=8.5 Hz, 1H), 7.37-7.33 (m, 2H), 7.28 (t, J=10.0 Hz, 2H), 6.29 (d, J=10.0 Hz, 1H), 5.99 (s, 1H), 5.41 (s, 2H), 4.96 (d, J=23.2 Hz, 2H), 4.84 (s, 1H), 4.62-4.56 (m, 2H), 4.39 (dt, J=13.4, 6.8 Hz, 3H), 4.14 (s, 1H), 3.99-3.85 (m, 6H), 3.63-3.57 (m, 2H), 3.56-3.44 (m, 16H), 3.41 (dd, J=11.7, 5.9 Hz, 2H), 3.02 (dd, J=13.4, 6.5 Hz, 1H), 2.94 (dd, J=13.1, 6.7 Hz, 1H), 2.44 (ddd, J=21.6, 10.2, 5.1 Hz, 4H), 2.36 (dd, J=13.5, 7.3 Hz, 1H), 2.08-1.98 (m, 5H), 1.90 (t, J=14.4 Hz, 2H), 1.83-1.67 (m, 6H), 1.61 (td, J=18.5, 7.5 Hz, 6H), 1.52-1.41 (m, 4H), 1.35 (dd, J=28.5, 14.5 Hz, 4H), 1.29-1.18 (m, 10H), 1.10 (dd, J=28.8, 11.6 Hz, 2H), 0.98 (s, 9H), 0.89 (s, 3H), 0.60 (s, 3H). ESI HRMS calcd C₉₁H₁₁₉Cl₂F₂N₁₁O₁₈S [M+H]⁺ m/z, 1794.7879; found 1795.7864

### EXAMPLE 15

### Preparation of Compound I-15

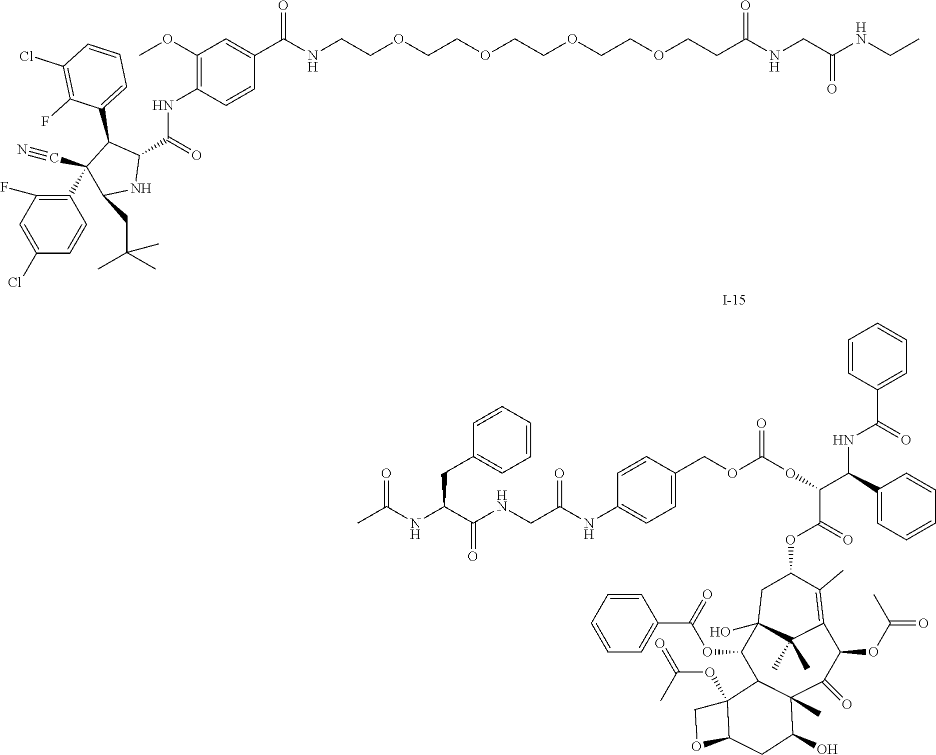

Following the method of example 1, 30 mg of a white solid compound I-15 was obtained by respectively substituting the Val-Cit with Gly-Gly-Phe-Gly, and the

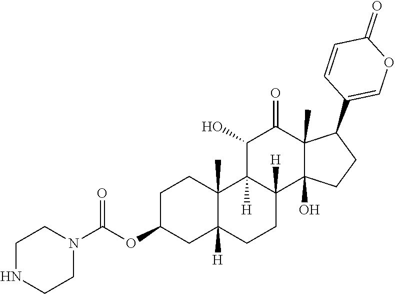

with paclitaxel. ¹H NMR (600 MHz, DMSO) δ10.42 (s, 1H), 9.95 (s, 1H), 9.29 (d, J=8.3 Hz, 1H), 8.51 (s, 1H), 8.41 (s, 1H), 8.32 (d, J=8.3 Hz, 1H), 8.17 (d, J=10.4 Hz, 2H), 8.04 (s, 1H), 7.99 (d, J=7.7 Hz, 2H), 7.83 (d, J=7.2 Hz, 2H), 7.73 (s, 2H), 7.63 (dt, J=16.7, 8.0 Hz, 5H), 7.55 (dd, J=17.7, 10.1 Hz, 4H), 7.49-7.43 (m, 6H), 7.39-7.31 (m, 5H), 7.26 (d, J=3.6 Hz, 4H), 7.19 (s, 2H), 6.31 (s, 1H), 5.83 (s, 1H), 5.54 (t, J=8.6 Hz, 1H), 5.42 (d, J=6.9 Hz, 1H), 5.37 (d, J=8.6 Hz, 1H), 5.14 (s, 2H), 4.97-4.89 (m, 2H), 4.66 (s, 1H), 4.60 (s, 2H), 4.52 (s, 1H), 4.39 (s, 1H), 4.12 (s, 1H), 4.02 (dd, J=15.6, 8.2 Hz, 2H), 3.92 (s, 4H), 3.81-3.74 (m, 1H), 3.70 (d, J=5.3 Hz, 2H), 3.60 (dd, J=17.4, 11.0 Hz, 4H), 3.52 (d, J=5.1 Hz, 5H), 3.49-3.44 (m, 8H), 3.41 (d, J=5.7 Hz, 2H), 3.08 (d, J=10.1 Hz, 1H), 2.87-2.80 (m, 1H), 2.39 (t, J=6.3 Hz, 2H), 2.27 (s, 3H), 2.12 (s, 3H), 2.00 (d, J=15.3 Hz, 1H), 1.81 (s, 4H), 1.68-1.61 (m, 2H), 1.51 (s, 4H), 1.27 (dd, J=21.0, 9.2 Hz, 5H), 1.03 (s, 3H), 1.00 (s, 3H), 0.98 (s, 9H). ¹³C NMR (151 MHz, DMSO-d₆) δ202.80, 171.91, 171.48, 171.18, 170.13, 169.73, 169.42, 169.34, 169.22, 168.08, 166.81, 165.99, 165.68, 154.27, 147.95, 139.64, 138.30, 137.44, 134.50, 133.95, 132.01, 131.41, 130.46, 130.40, 130.04, 129.96, 129.81, 129.74, 129.60, 129.20, 129.13, 129.05, 128.78, 128.54, 128.03, 127.86, 126.74, 126.08, 125.72, 120.51, 119.46, 117.77, 117.47, 110.06, 84.07, 80.73, 77.60, 77.14, 75.75, 75.17, 74.95, 71.63, 70.86, 70.19, 70.15, 70.07, 69.95, 69.43, 67.10, 65.07, 63.70, 57.86, 56.26, 54.71, 54.38, 50.62, 46.54, 44.34, 43.40, 43.18, 42.58, 42.29, 37.85, 36.99, 36.32, 34.83, 31.60, 30.53, 30.28, 29.95, 29.47, 29.27, 26.80, 22.99, 21.81, 21.12, 14.36, 10.22. ESI HRMS calcd C₁₁₂H₁₂₄Cl₂F₂N₁₀O₂₈ [M+H]⁺ m/z, 2165.7932; found 2165.8004.

### EXAMPLE 16

### Preparation of Compound I-16

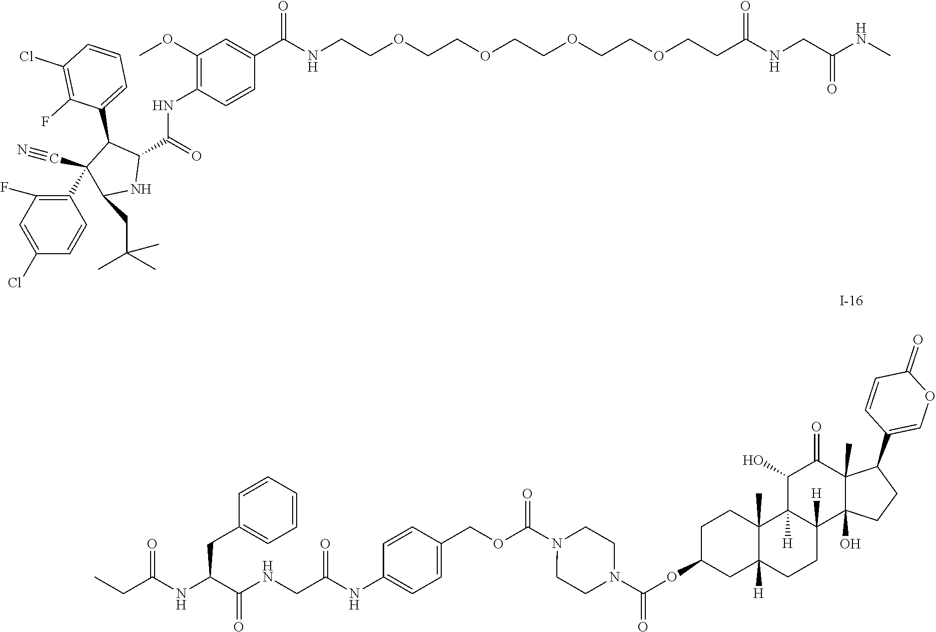

Following the method of example 1, 29 mg of a white solid compound I-16 was obtained by substituting the Val-Cit with Gly-Gly-Phe-Gly. ¹H NMR (600 MHz, DMSO) δ10.40 (s, 1H), 9.85 (s, 1H), 8.48 (t, J=5.6 Hz, 1H), 8.38 (t, J=5.8 Hz, 1H), 8.31 (d, J=8.4 Hz, 1H), 8.18-8.13 (m, 2H), 8.02 (t, J=5.6 Hz, 1H), 7.81 (dd, J=9.9, 2.5 Hz, 1H), 7.73 (t, J=6.9 Hz, 1H), 7.59 (dd, J=20.0, 9.9 Hz, 5H), 7.53 (t, J=7.7 Hz, 1H), 7.49 (d, J=8.5 Hz, 1H), 7.39 (t, J=8.5 Hz, 1H), 7.37-7.30 (m, 4H), 7.26 (d, J=4.3 Hz, 4H), 7.18 (dd, J=8.5, 4.4 Hz, 1H), 6.33 (d, J=9.9 Hz, 1H), 5.03 (s, 2H), 4.96 (s, 1H), 4.86 (s, 1H), 4.62-4.56 (m, 2H), 4.55-4.48 (m, 2H), 4.36 (s, 1H), 4.28 (dd, J=10.9, 4.8 Hz, 1H), 4.00-3.89 (m, 6H), 3.81 (ddd, J=22.9, 16.6, 5.8 Hz, 2H), 3.69 (dd, J=5.5, 2.3 Hz, 2H), 3.61 (dt, J=12.9, 6.0 Hz, 3H), 3.53 (t, J=5.8 Hz, 6H), 3.50-3.44 (m, 8H), 3.41 (dd, J=15.6, 9.8 Hz, 10H), 3.07 (dd, J=13.7, 4.6 Hz, 1H), 2.83 (dd, J=13.6, 9.7 Hz, 1H), 2.45 (d, J=13.7 Hz, 1H), 2.38 (t, J=6.6 Hz, 2H), 2.04-1.95 (m, 1H), 1.87 (dd, J=29.5, 14.3 Hz, 4H), 1.73 (t, J=11.5 Hz, 2H), 1.69-1.58 (m, 4H), 1.43 (d, J=14.5 Hz, 1H), 1.28 (t, J=9.2 Hz, 2H), 1.22 (d, J=17.9 Hz, 4H), 1.10 (s, 3H), 0.98 (s, 9H), 0.80 (s, 3H). ¹³C NMR (151 MHz, DMSO-d₆) δ213.75, 171.89, 171.19, 169.75, 166.02, 150.70, 147.99, 138.98, 138.30, 131.43, 130.48, 129.77, 129.61, 129.07, 128.92, 128.56, 126.75, 125.75, 121.33, 120.52, 119.53, 117.78, 117.52, 115.09, 110.10, 84.64, 73.62, 71.48, 70.22, 70.18, 70.10, 69.97, 69.46, 67.13, 66.65, 65.09, 62.64, 56.28, 54.80, 50.66, 44.38, 43.20, 42.61, 42.33, 37.81, 36.98, 36.35, 33.13, 32.39, 31.09, 30.55, 29.97, 29.47, 28.12, 26.74, 25.93, 23.85, 21.74, 17.74. ESI HRMS calcd C₉₄H₁₁₃Cl₂F₂N₁₁O₂₁ [M+H]⁺ m/z, 1840.7536; found 1840.7520

### EXAMPLE 17

### Preparation of Compound I-17

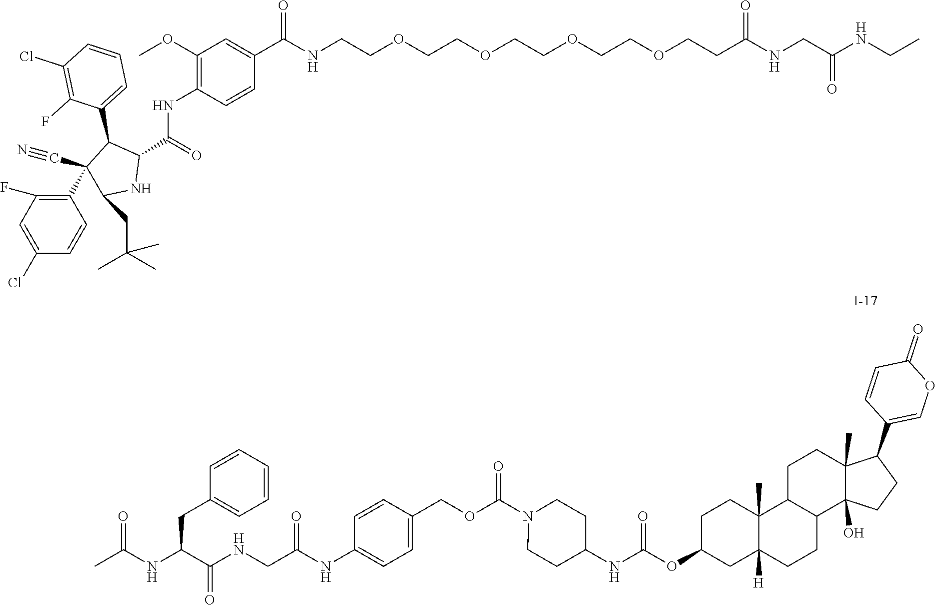

Following the method of example 1, 27 mg of a light green solid compound I-17 was obtained by respectively substituting the

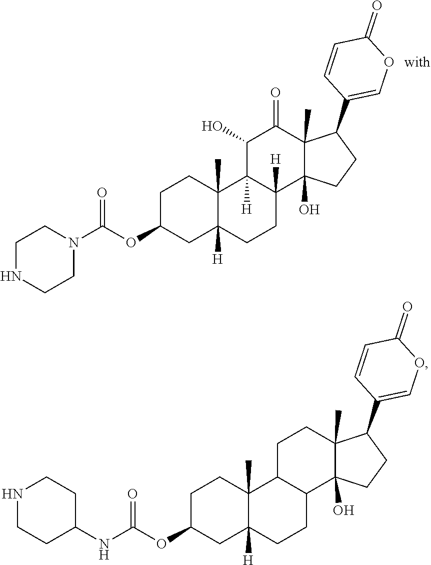

and the Val-Cit with Gly-Gly-Phe-Gly. ¹H NMR (600 MHz, DMSO) δ10.41 (s, 1H), 9.86 (s, 1H), 8.50 (s, 1H), 8.40 (s, 1H), 8.32 (d, J=8.4 Hz, 1H), 8.17 (d, J=6.8 Hz, 2H), 8.04 (s, 1H), 7.94 (d, J=9.9 Hz, 1H), 7.74 (t, J=6.9 Hz, 1H), 7.63-7.56 (m, 4H), 7.56-7.47 (m, 3H), 7.42-7.33 (m, 3H), 7.30 (d, J=8.3 Hz, 2H), 7.26 (d, J=3.8 Hz, 5H), 7.19 (d, J=3.6 Hz, 1H), 6.29 (d, J=9.6 Hz, 1H), 4.94 (s, 2H), 4.84 (s, 1H), 4.60 (s, 2H), 4.51 (s, 1H), 4.38 (s, 1H), 4.16 (s, 1H), 3.99-3.83 (m, 9H), 3.78 (dd, J=16.8, 5.6 Hz, 1H), 3.70 (d, J=4.6 Hz, 2H), 3.59 (t, J=6.3 Hz, 3H), 3.52 (d, J=4.9 Hz, 7H), 3.50-3.45 (m, 9H), 3.41 (d, J=5.5 Hz, 2H), 3.17 (s, 1H), 3.07 (d, J=10.1 Hz, 1H), 2.86 (dd, J=38.0, 27.4 Hz, 3H), 2.46 (s, 1H), 2.39 (t, J=6.2 Hz, 2H), 2.08-2.00 (m, 2H), 1.92 (t, J=13.0 Hz, 1H), 1.77 (d, J=15.1 Hz, 4H), 1.63 (dd, J=29.2, 12.9 Hz, 5H), 1.50 (t, J=18.2 Hz, 4H), 1.39 (s, 1H), 1.32 (dd, J=27.1, 16.0 Hz, 4H), 1.17 (dd, J=29.7, 13.9 Hz, 3H), 0.98 (s, 9H), 0.89 (s, 3H), 0.60 (s, 3H). ¹³C NMR (151 MHz, DMSO-d₆) δ171.92, 171.20, 169.75, 169.40, 167.97, 166.01, 161.82, 155.80, 154.48, 149.67, 147.89, 138.92, 138.30, 132.26, 131.41, 130.47, 130.06, 129.75, 129.61, 129.21, 129.06, 128.56, 126.75, 126.08, 125.73, 123.21, 120.52, 119.38, 117.78, 117.48, 114.66, 110.07, 83.88, 70.97, 70.21, 70.09, 69.96, 69.45, 67.12, 65.49, 65.09, 63.88, 63.67, 56.26, 55.37, 54.79, 50.62, 48.52, 47.98, 44.36, 43.17, 42.59, 42.30, 41.67, 37.81, 37.43, 36.33, 35.32, 32.48, 32.04, 31.00, 30.73, 30.55, 29.96, 29.48, 28.95, 26.79, 25.19, 24.26, 21.58, 21.49, 17.13. ESI HRMS calcd C₉₅H₁₁₇Cl₂F₂N₁₁O₁₉ [M+H]⁺ m/z, 1824.7872; found 1824.7945.

### EXAMPLE 18

### Preparation of Compound I-18

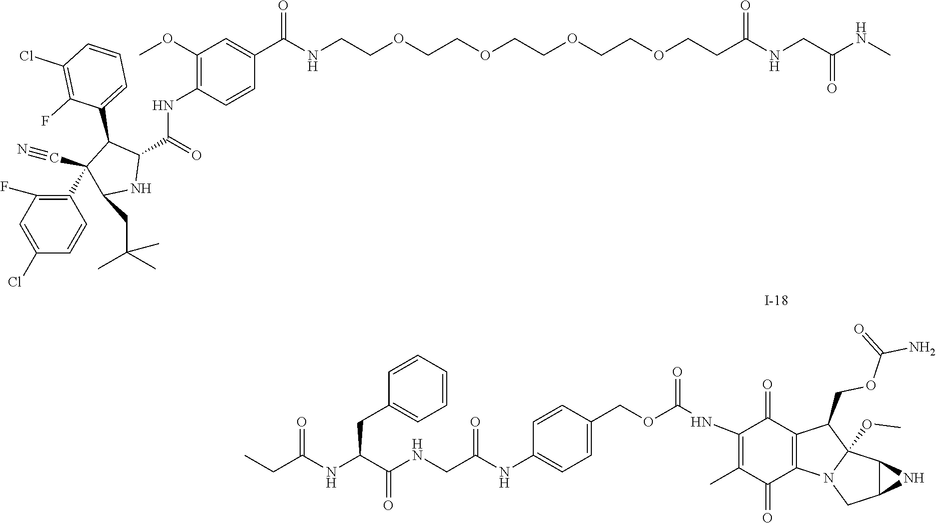

Following the method of example 1, 30 mg of a purple solid I-18 was obtained by respectively substituting the

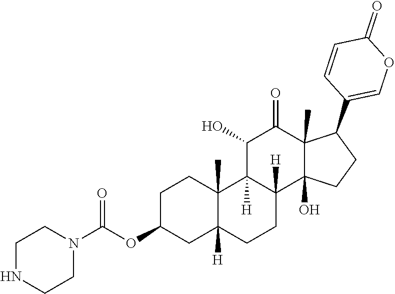

with mitomycin C, and the Val-Cit with Gly-Gly-Phe-Gly. ¹H NMR (600 MHz, DMSO) δ10.41 (s, 1H), 9.88 (s, 1H), 8.50 (t, J=5.5 Hz, 1H), 8.39 (t, J=5.8 Hz, 1H), 8.32 (d, J=8.4 Hz, 1H), 8.16 (dd, J=10.6, 5.1 Hz, 2H), 8.03 (t, J=5.7 Hz, 1H), 7.74 (t, J=6.9 Hz, 1H), 7.59 (dd, J=9.0, 5.4 Hz, 4H), 7.54 (t, J=7.6 Hz, 1H), 7.49 (dd, J=8.4, 1.6 Hz, 1H), 7.40 (t, J=8.5 Hz, 1H), 7.37-7.30 (m, 4H), 7.28-7.23 (m, 4H), 7.18 (dq, J=8.7, 4.3 Hz, 1H), 7.06 (s, 2H), 6.66 (s, 2H), 4.97 (dd, J=36.3, 12.3 Hz, 2H), 4.73 (dd, J=10.6, 4.6 Hz, 1H), 4.62-4.56 (m, 2H), 4.54-4.48 (m, 1H), 4.38 (s, 1H), 4.24 (d, J=13.5 Hz, 1H), 3.99-3.89 (m, 6H), 3.85 (dd, J=16.5, 5.7 Hz, 1H), 3.77 (dd, J=16.8, 6.0 Hz, 1H), 3.70 (d, J=5.7 Hz, 2H), 3.64-3.56 (m, 4H), 3.52 (dd, J=10.7, 5.5 Hz, 8H), 3.49-3.44 (m, 8H), 3.41 (dd, J=12.4, 6.5 Hz, 3H), 3.13 (s, 3H), 3.07 (dd, J=13.9, 4.4 Hz, 1H), 2.83 (dd, J=13.9, 9.9 Hz, 1H), 2.39 (t, J=6.5 Hz, 2H), 1.68-1.61 (m, 4H), 1.27 (d, J=13.4 Hz, 1H), 0.98 (s, 9H). ¹³C NMR (151 MHz, DMSO-d₆) δ177.09, 176.26, 171.88, 171.49, 171.19, 169.73, 169.35, 167.96, 166.01, 160.69, 157.02, 154.94, 149.24, 147.96, 139.17, 138.30, 131.43, 130.96, 130.47, 130.06, 129.75, 129.60, 129.45, 129.06, 128.54, 126.74, 126.08, 125.73, 120.51, 119.40, 117.93, 117.77, 117.48, 110.07, 110.02, 105.79, 103.36, 70.20, 70.16, 70.08, 69.95, 69.44, 67.94, 67.11, 65.08, 63.67, 61.00, 56.27, 55.36, 54.73, 50.62, 49.75, 48.98, 44.35, 43.40, 43.15, 42.58, 42.28, 37.84, 36.32, 30.54, 29.96, 29.48, 8.79. ESI HRMS calcd C₈₀H₉₁Cl₂F₂N₁₃O₁₉ [M+H]⁺ m/z, 1646.5899; found 1646.5972.

### EXAMPLE 19

### In-Vitro Antitumor Activity Test of Compounds of the Present Disclosure

A part of the compounds prepared in the examples of the present disclosure were subjected to a tumor cell proliferation inhibition test by using a CCK-8 method. A cell line HCT116 (human colon carcinoma cells) and SW1990 (human pancreatic carcinoma cells) were purchased from Shanghai Meixuan Technology Co., Ltd. Samples (a part of the compounds prepared in the examples) were dissolved in DMSO (Merck), prepared into a concentration of 10 mM, and finally triply diluted in DMEM or McCoy's 5A 1640 medium to 8 concentration gradients. A cell culture solution was collected when a cell culture met a test requirement, 10 μL of the cell culture solution was uniformly coated on a cell counting plate, cells were counted for 3 times under a microscope, and a cell density was calculated by taking an average value; and a 96-well plate (Corning, #3599) was taken, 200 μL of the cell culture solution was added into each well for cell inoculation at a cell concentration of 6×10³ cells per well, and the inoculated 96-well plate was incubated in a cell incubator containing 5% CO₂ at 37° C. for 24 h. An old culture medium was removed by suction, 200 μL of the triply diluted sample DMEM or McCoy's 5A 1640 medium at an initial concentration of 10 μmol/L was added to each well, the DMSO content was controlled to be 1%, three duplicate wells were set, PBS was used as a blank control, and the plate was incubated in the incubator for 72 h. The original DMEM or McCoy's 5A 1640 medium in the 96-well plate and a PBS buffer in a final row were sucked out, a prepared CCK-8 solution (90% DMEM or McCoy's 5A 1640 medium+10% CCK-8) was added, the mixture was incubated in an incubator, and an absorbance was read by using a Biotek microplate reader. Cell growth inhibition rate IC %=(blank control well OD value−administration well OD value)/blank control well OD value×100%. According to IC % values of each concentration, a drug concentration at which each compound inhibited a cell growth by 50%, i.e., IC₅₀, was calculated by a linear regression using a GraphPad software.

The test results were shown in Table 1, wherein the samples refer to the compounds prepared in the corresponding examples.

TABLE 1
In-vitro antitumor activity of part
of compounds of examples

| | IC₅₀ (μM) | |
|---|---|---|
| Number | HCT116 | SW1990 |
| Idasanutlin | 1.059 | 2.975 |
| I-1 | 0.034 | 0.007 |
| I-3 | 2.092 | 1.753 |
| I-4 | 0.728 | 0.576 |
| I-6 | 0.542 | >50 |
| I-15 | 6.111 | 7.606 |
| I-16 | 0.210 | 0.022 |
| I-17 | 0.898 | 0.287 |
| I-18 | 0.296 | 1.4027 |

### EXAMPLE 20

### In-Vitro Antitumor Activity Test of Compounds of the Present Disclosure

A part of the compounds of the examples of the present disclosure were subjected to a tumor cell proliferation inhibition test by using an MTT method (e.g. Pharmacological Research Method of New Drug, 2007: 242-243, edited by Lu Qiujun).

A cell strain was selected from KYSE-150 (human esophageal squamous carcinoma cells), MGC803 (human gastric carcinoma cells), and SHG44 (human glioma cells), and frozen and passaged by a pharmacological laboratory of Shanghai Institute of Pharmaceutical Industry. A culture solution was DMEM+10% FBS+double antibody.

In-vitro activity test: 100 μL of a cell suspension at a concentration of 4-5×10⁴/mL was added to each well of a 96-well plate, and placed in a 5% CO₂ incubator at 37° C. After 24 h, a sample solution was added at 10 μL/well, double duplicate wells were set, and a reaction was performed at 37° C. with 5% CO₂ for 72 h. 20 μL of 5 mg/ml of an MTT solution was added into each well and reacted for 4 h, a dissolving solution was added at 100 μL/well, the mixture was placed in an incubator, and after dissolution, an OD value at 570 nm was measured with a full-wavelength multifunctional microplate reader. An inhibition rate (IR) and a median inhibitory concentration (IC₅₀) were calculated by using an Excel software.

The inhibition rate (IR %) of the drug on cell growth was calculated according to the following equation:

$$IR ( \% ) = ( 1 - \frac{Average OD of sample group}{Average OD of control group} ) \times 100 \%$$

The test results were shown in Table 2, wherein the samples refer to the compounds prepared in the corresponding examples.

TABLE 2
In-vitro antitumor activity of part
of compounds of examples

| | IC₅₀ (μM) | | |
|---|---|---|---|
| Number | KYSE-150 | MGC803 | SHG44 |
| I-1 | 0.040 | 0.009 | 0.003 |
| I-3 | 4.15 | 0.705 | 0.831 |
| I-4 | 1.06 | 1.67 | 0.319 |
| I-6 | 19.38 | >50 | >50 |
| I-15 | 0.559 | 3.56 | 0.550 |
| I-16 | 0.243 | 0.402 | 0.041 |
| I-17 | 1.89 | 1.62 | 0.204 |
| I-18 | 5.34 | 2.11 | 6.84 |

### EXAMPLE 21

### Antitumor Activity Test of Compounds of the Present Disclosure

A part of the compounds prepared in the examples of the present disclosure were subjected to a tumor cell proliferation inhibition test by using a CCK-8 method. Cell lines HCT116 (human colon cancer cells), MGC803 (human gastric cancer cells), and MIA PACA-2 (human pancreatic cancer cells) were purchased from Shanghai Meixuan Technology Co., Ltd. Samples (a part of the compounds prepared in the examples) were dissolved in DMSO (Merck), prepared into a concentration of 10 mM, and finally triply diluted in DMEM or McCoy's 5A 1640 medium to 8 concentration gradients. A cell culture solution was collected when a cell culture met a test requirement, 10 μL of the cell culture solution was uniformly coated on a cell counting plate, cells were counted for 3 times under a microscope, and a cell density was calculated by taking an average value; and a 96-well plate (Corning, #3599) was taken, 200 μL of the cell culture solution was added into each well for cell inoculation at a cell concentration of 6×10³ cells per well, and the inoculated 96-well plate was incubated in a cell incubator containing 5% CO₂ at 37° C. for 24 h. An old culture medium was removed by suction, 200 μL of the triply diluted sample DMEM or McCoy's 5A 1640 medium at an initial concentration of 10 μmol/L was added to each well, the DMSO content was controlled to be 1%, three duplicate wells were set, PBS was used as a blank control, and the plate was incubated in the incubator for 72 h. The original DMEM or McCoy's 5A 1640 medium in the 96-well plate and a PBS buffer in a final row were sucked out, a prepared CCK-8 solution (90% DMEM or McCoy's 5A 1640 medium+10% CCK-8) was added, the mixture was incubated in an incubator, and an absorbance was read by using a Biotek microplate reader. Cell growth inhibition rate IC %=(blank control well OD value−administration well OD value)/blank control well OD value×100%. According to IC % values of each concentration, a drug concentration at which each compound inhibited a cell growth by 50%, i.e., IC₅₀, was calculated by a linear regression using a GraphPad software.

The test results were shown in Table 3, wherein the samples refer to the compounds prepared in the corresponding examples.

TABLE 3
In-vitro antitumor activity of part
of compounds of examples

| | IC₅₀ (μM) | | |
|---|---|---|---|
| Number | HCT116 | MGC803 | MIA PACA-2 |
| I-2 | 0.026 | 0.054 | 0.212 |
| I-5 | 0.236 | 0.116 | 0.442 |
| I-7 | 1.287 | 0.292 | 3.534 |
| I-8 | 0.012 | 0.012 | 0.087 |
| I-10 | 0.395 | 0.307 | 0.443 |
| I-11 | 0.027 | 0.010 | 0.068 |
| I-12 | 5.265 | 0.626 | 0.542 |
| I-13 | 0.014 | 0.014 | 0.024 |
| I-14 | 0.483 | 0.249 | 0.670 |
| Idasanutlin | 0.110 | 1.091 | >10 |

The experimental results showed that the compounds of the present disclosure had a good antitumor activity and showed an excellent activity on cell strains such as colon cancer, pancreatic cancer, esophageal squamous carcinoma, gastric cancer, glioma and the like. Proliferation inhibition activities IC₅₀ of the compounds such as I-1, I-2, I-4, I-8, I-11, I-13, I-14, I-15, I-16, I-17 and the like on most tumor cell strains were all between several to hundreds of nanomoles. Therefore, the compounds and salts thereof of the present disclosure may be used in the preparation of an antitumor drug.

### EXAMPLE 22

### In-Vivo Antitumor Activity Test of Compounds I-1, I-4, I-16, and I-17 of the Present Disclosure in Human Stromal Tumor SHG44

Animals: BALB/C nude mice (SPF grade), male, and 18-20 g. The compounds were firstly added with DMSO at a total amount of 4%, then added with Tween-80 at a total amount of 2% for assisting dissolution, then added with PEG300 at a total amount of 5%, and finally added with sterilized normal saline to be prepared into a solution at 1 mg/mL. Idasanutlin was firstly added with DMSO at a total amount of 4%, then added with Tween-80 at a total amount of 2% for assisting dissolution, then added with PEG300 at a total amount of 5%, and finally added with sterilized normal saline to be prepared into a solution at 2.5 mg/mL.

A well-grown human stromal tumor SHG44 mass was taken and aseptically cut into uniform small blocks of about 3 mm in size, and a right axilla of each mouse was subcutaneously inoculated with one block through a trocar. On day 9 after the inoculation, an average volume of the tumor mass was found to be about 130 mm³. The animals were regrouped according to a tumor size. The animals with too large and too small tumors were sifted out. An average tumor volume in each group was approximately the same. An administration was started according to the following solution with an administration volume of 0.2 mL/20 g body weight. A long diameter a (mm) of the tumor and a short diameter b (mm) vertical to the tumor were measured by a digital display electronic caliper 2 times a week from day 9 of the inoculation. A tumor volume was calculated by the formula: TV=ab²/2, and a relative tumor volume was calculated by the formula: RTV=Vt/Vo, wherein Vo was the obtained tumor volume measured at a cage separation (i.e. d1) and Vt was the tumor volume measured at each time. The animals were sacrificed 30 days after the inoculation (d22) and weighed, tumor mass was obtained by dissection and weighed, and the results were determined according to the following formula:

$$Tumor inhibition rate ( \% ) = \frac{\begin{matrix}Average RTV of control group - \\ Average RTV of adminstration group\end{matrix}}{Average RTV of control group} \times 100 \%$$

TABLE 4
In-vivo antitumor activity of compounds I-1, I-4, I-16, and I-17 in human stromal tumor SHG44

| | | | Number of | Body weight | | |
|---|---|---|---|---|---|---|
| | | | animals | of animals | | Tumor |
| | Dose | Administration | Beginning | (After a tumor | RTV | inhibition |
| Groups | (mg/kg) | Solution | Ending | is removed) | (d22) | rate % |
| Control (NS) | 10 mL/kg | | 8 8 | 24.12 ± 1.27 | 4.88 ± 0.95 | |
| I-1 | 10 | Ipx5 each | 6 6 | 24.78 ± 2.38 | 2.34 ± 0.32** | 52.03 |
| | | week for 2 | | | | |
| | | continuous | | | | |
| | | weeks | | | | |
| I-4 | 10 | Ipx5 each | 6 6 | 26.10 ± 2.00 | 2.28 ± 0.31** | 53.31 |
| | | week for 2 | | | | |
| | | continuous | | | | |
| | | weeks | | | | |
| I-16 | 10 | Ipx5 each | 6 6 | 25.13 ± 0.97 | 2.48 ± 0.96** | 49.23 |
| | | week for 2 | | | | |
| | | continuous | | | | |
| | | weeks | | | | |
| I-17 | 10 | Ipx5 each | 6 6 | 25.60 ± 0.98* | 3.11 ± 0.37** | 36.30 |
| | | week for 2 | | | | |
| | | continuous | | | | |
| | | weeks | | | | |
| Idasanutlin | 25 | Igx14 | 6 6 | 24.47 ± 1.66 | 2.95 ± 0.41** | 39.59 |

Compared with control group, **indicates P < 0.01.

The experimental results showed that the compounds I-1, I-4, I-16, and I-17 had a good in-vivo antitumor activity of the human stromal tumor SHG44, such that the compounds and salts thereof of the present disclosure may be used in the preparation of an antitumor drug.

### EXAMPLE 23

### In-Vivo Antitumor Activity Test of COmpounds I-1 and I-4 of the Present Disclosure in Human Gastric Carcinoma MGC803

Animals: BALB/C nude mice (SPF grade), male, and 18-20 g. The compounds were firstly added with DMSO at a total amount of 4%, then added with Tween-80 at a total amount of 2% for assisting dissolution, then added with PEG300 at a total amount of 5%, and finally added with sterilized normal saline to be prepared into a solution at 1 mg/mL. Idasanutlin was firstly added with DMSO at a total amount of 4%, then added with Tween-80 at a total amount of 2% for assisting dissolution, then added with PEG300 at a total amount of 5%, and finally added with sterilized normal saline to be prepared into a solution at 2.5 mg/mL.

A well-grown human gastric carcinoma MGC803 tumor mass was taken and aseptically cut into uniform small blocks of about 3 mm in size, and a right axilla of each mouse was subcutaneously inoculated with one block through a trocar. On day 9 after the inoculation, an average volume of the tumor mass was found to be about 130 mm³. The animals were regrouped according to a tumor size. The animals with too large and too small tumors were sifted out. An average tumor volume in each group was approximately the same. An administration was started according to the following solution with an administration volume of 0.2 mL/20 g body weight. A long diameter a (mm) of the tumor and a short diameter b (mm) vertical to the tumor were measured by a digital display electronic caliper 2 times a week from day 9 of the inoculation. A tumor volume was calculated by the formula: TV=ab²/2, and a relative tumor volume was calculated by the formula: RTV=Vt/Vo, wherein Vo was the obtained tumor volume measured at a cage separation (i.e. d1) and Vt was the tumor volume measured at each time. The animals were sacrificed 30 days after the inoculation (d22) and weighed, tumor mass was obtained by dissection and weighed, and the results were determined according to the following formula:

$$Tumor inhibition rate ( \% ) = \frac{\begin{matrix}Average RTV of control group - \\ Average RTV of adminstration group\end{matrix}}{Average RTV of control group} \times 100 \%$$

TABLE 5
In-vivo antitumor activity of compounds I-1 and I-4 in human gastric carcinoma MGC803

| | | | Number of | Body weight | | |
|---|---|---|---|---|---|---|
| | | | animals | of animals | | Tumor |
| | Dose | Administration | Beginning | (After a tumor | RTV | inhibition |
| Groups | (mg/kg) | Solution | Ending | is removed) | (d22) | rate % |
| Control (NS) | 10 mL/kg | | 8 8 | 25.47 ± 1.69 | 3.99 ± 0.61 | |
| I-1 | 10 | Ipx5 each | 6 6 | 26.15 ± 1.14 | 2.45 ± 0.54** | 38.54 |
| | | week for 2 | | | | |
| | | continuous | | | | |
| | | weeks | | | | |
| I-4 | 10 | Ipx5 each | 6 6 | 24.95 ± 1.62 | 1.82 ± 0.48** | 54.50 |
| | | week for 2 | | | | |
| | | continuous | | | | |
| | | weeks | | | | |
| Idasanutlin | 25 | Igx14 | 6 6 | 23.58 ± 1.56 | 2.74 ± 0.81** | 31.36 |

Compared with control group, **indicates P < 0.01.

The experimental results showed that the compounds I-1 and I-4 had a good in-vivo antitumor activity of the human gastric carcinoma MGC803, such that the compounds and salts thereof of the present disclosure may be used in the preparation of an antitumor drug.

### EXAMPLE 24

### In-Vivo Antitumor Activity Test of Compounds I-1 and I-4 of the Present Disclosure in Human Esophageal Cancer KYSE-150

Animals: BALB/C nude mice (SPF grade), male, and 18-20 g. The compounds were firstly added with DMSO at a total amount of 4%, then added with Tween-80 at a total amount of 2% for assisting dissolution, then added with PEG300 at a total amount of 5%, and finally added with sterilized normal saline to be prepared into a solution at 1 mg/mL. Idasanutlin was firstly added with DMSO at a total amount of 4%, then added with Tween-80 at a total amount of 2% for assisting dissolution, then added with PEG300 at a total amount of 5%, and finally added with sterilized normal saline to be prepared into a solution at 2.5 mg/mL.

A well-grown human esophageal cancer KYSE-150 tumor mass was taken and aseptically cut into uniform small blocks of about 3 mm in size, and a right axilla of each mouse was subcutaneously inoculated with one block through a trocar. On day 9 after the inoculation, an average volume of the tumor mass was found to be about 130 mm³. The animals were regrouped according to a tumor size. The animals with too large and too small tumors were sifted out. An average tumor volume in each group was approximately the same. An administration was started according to the following solution with an administration volume of 0.2 mL/20 g body weight. A long diameter a (mm) of the tumor and a short diameter b (mm) vertical to the tumor were measured by a digital display electronic caliper 2 times a week from day 9 of the inoculation. A tumor volume was calculated by the formula: TV=ab²/2, and a relative tumor volume was calculated by the formula: RTV=Vt/Vo, wherein Vo was the obtained tumor volume measured at a cage separation (i.e. d1) and Vt was the tumor volume measured at each time. The animals were sacrificed 30 days after the inoculation (d22) and weighed, tumor mass was obtained by dissection and weighed, and the results were determined according to the following formula:

$$Tumor inhibition rate ( \% ) = \frac{\begin{matrix}Average RTV of control group - \\ Average RTV of adminstration group\end{matrix}}{Average RTV of control group} \times 100 \%$$

TABLE 6
In-vivo antitumor activity of compounds I-1 and I-4 in human esophageal cancer KYSE-150

| | | | Number of | Body weight | | |
|---|---|---|---|---|---|---|
| | | | animals | of animals | | Tumor |
| | Dose | Administration | Beginning | (After a tumor | RTV | inhibition |
| Groups | (mg/kg) | Solution | Ending | is removed) | (d22) | rate % |
| Control (NS) | 10 mL/kg | | 8 8 | 24.93 ± 1.59 | 9.82 ± 1.55 | |
| I-1 | 10 | Ipx5 each | 6 6 | 24.51 ± 1.40 | 4.87 ± 0.92** | 50.45 |
| | | week for 2 | | | | |
| | | continuous | | | | |
| | | weeks | | | | |
| I-4 | 10 | Ipx5 each | 6 6 | 24.99 ± 1.48 | 4.47 ± 0.86** | 54.48 |
| | | week for 2 | | | | |
| | | continuous | | | | |
| | | weeks | | | | |
| Idasanutlin | 25 | Igx14 | RG7388 | 24.03 ± 0.95 | 4.79 ± 0.41** | 51.17 |

Compared with control group, **indicates P < 0.01.

The experimental results showed that the compounds I-1 and I-4 had a good in-vivo antitumor activity of the human esophageal cancer KYSE-150, such that the compounds and salts thereof of the present disclosure may be used in the preparation of an antitumor drug.

### EXAMPLE 25

### Test of Effect of Compound I-1 of the Present Disclosure on hERG Potassium Channel

CHO-hERG cells were cultured in a 175-cm² culture flask. After a cell density reached a culture solution was removed, and the cells were washed once with 7 mL of phosphate buffered saline (PBS) and digested with 3 mL of detachin. After the complete digestion, 7 mL of a culture solution was added for neutralization, the mixture was centrifuged, a supernatant was sucked away, and then 5 mL of the culture solution was added for resuspension so as to ensure that the cell density was 2-5×10⁶/mL.

Forming processes of single-cell high-impedance sealing and whole-cell mode were both automatically completed by a Qpatch instrument. After a whole-cell recording mode was obtained, cells were clamped at −80 millivolts. Before a depolarization stimulation of +40 millivolt for 5 seconds was given, a preset voltage of −50 millivolts was firstly given for 50 milliseconds, then repolarization was performed to −50 millivolt and maintained for 5 seconds, and then the voltage returned to −80 millivolts. The voltage stimulus was applied every 15 seconds, recording was performed for 2 minutes, extracellular fluid was given, recording was performed for 5 minutes, then an administration process was started, each test concentration was given for 2.5 minutes from a lowest test concentration of a compound concentrations, and after all the concentrations were continuously given, 3 μM of cisapride was given to a positive control compound. At least 3 cells (n≥3) were tested per concentration.

20 mM of a compound stock was diluted in DMSO, and 10 μL of 20 mM of the compound stock was added to 20 μL of a DMSO solution and triply serially diluted to 6 DMSO concentrations. 4 μL of the compounds with 6 DMSO concentrations were respectively taken and added into 396 μL of extracellular fluid, the mixture was diluted 100-fold to 6 intermediate concentrations, and then 80 μL of the compounds with 6 intermediate concentrations were respectively taken and added to 320 μL of the extracellular fluid and diluted 5-fold to a final concentration to be tested. A highest test concentration was 40 μM and the 6 concentrations were 40, 13.33, 4.44, 1.48, 0.49, and 0.16 μM in sequence. In the final concentration, the content of DMSO did not exceed 0.2%. The concentration of DMSO had no effect on an hERG potassium channel. The whole dilution process of the compound was completed by a Bravo instrument. Experimental data was analyzed by a GraphPad Prism 5.0 software.

TABLE 7
Test of hERG potassium channel
activity of compound

| Compound | IC₅₀ (μM) |
|---|---|
| I-1 | >40 |
| Cisapride | 0.042 |

The above experimental results indicated that the compounds of the present disclosure had a lower hERG potassium channel inhibitory activity, indicating a relatively low cardiotoxicity.

The above is only preferred examples of the present disclosure and are not intended to limit the present disclosure in any form. Although the present disclosure has been disclosed in the preferred examples, the preferred examples are not intended to limit the present disclosure. Without departing from the scope of the technical solution of the present disclosure, a technician familiar with the patent may make some changes or modifications to equivalent examples with equivalent changes by using the technical content indicated above. However, any simple modifications, equivalent changes, and modifications made to the above examples based on the technical essence of the present disclosure without departing from the content of the technical solution of the present disclosure, still fall within the scope of the solution of the present disclosure.

1. A small-molecule conjugate or a pharmaceutical salt thereof, wherein a structure of the small-molecule conjugate is shown in a general formula I: 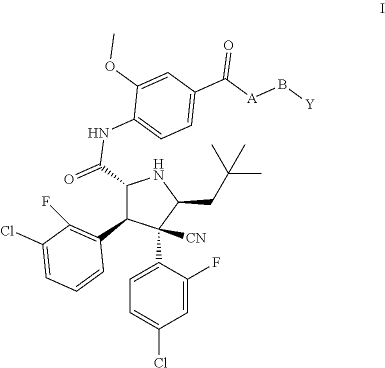
A is a spacer linking group;
B is a releasable linking group; and
Y is a drug;
the A is selected from: 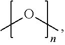
wherein n is 1-10, 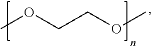
wherein n is 1-10, sugar, alkylene, 1-alkylene succinimide-3-yl, 1-(carbonylalkyl)succinimide-3-yl, or a combination thereof; wherein the A may be substituted by at least one substituent selected from alkyl, alkoxy, alkoxyalkyl, hydroxyl, hydroxyalkyl, amino, aminoalkyl, alkylaminoalkyl, dialkylaminoalkyl, mercaptoalkyl, alkylthioalkyl, aryl, substituted aryl, aralkyl, substituted aralkyl, heteroaryl, substituted heteroaryl, carboxyl, carboxyalkyl, carboxylate alkyl ester, guanidinoalkyl, or carbonyl or acylamino or acylaminoalkyl substituted with an amino acid and a derivative thereof, and a peptide; and
the B comprises at least one linking group formed by an amino acid selected from a natural amino acid or a non-natural a amino acid; or the B is a linking group of a cleavable bond under a physiological condition.

2. The small-molecule conjugate or a pharmaceutical salt thereof according to claim 1, wherein the B is selected from one or a combination of the following structures: 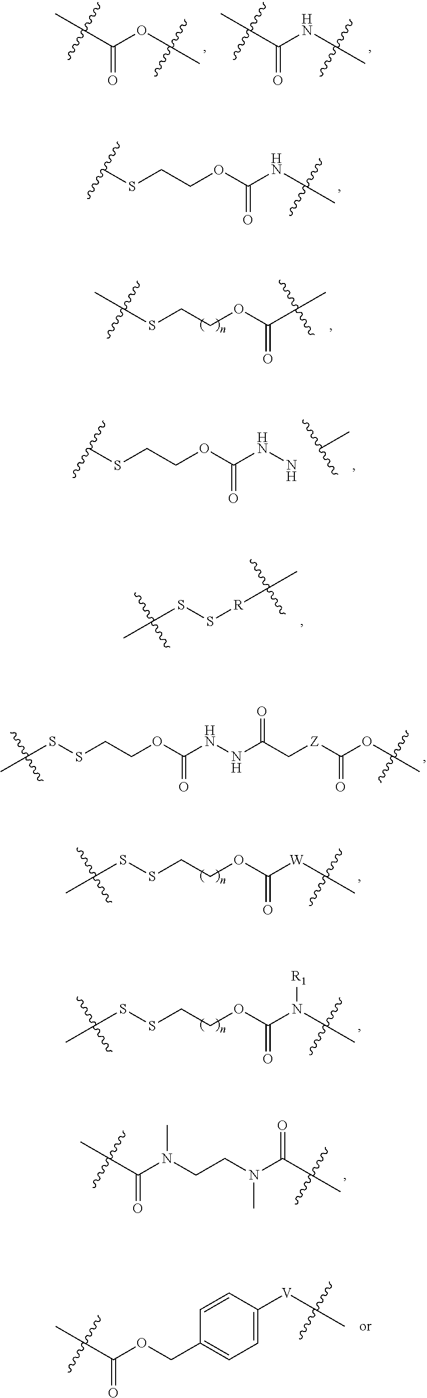 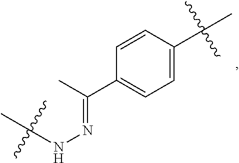
wherein n is 0, 1, 2, 3, or 4; R is —(CH₂)n— and —OOCCH₂—, and n is 1-10;
Z is —O—, —CH₂— or —NH—; W is O or S; and V is —NH—, —O—, or —S—; and
R₁ is hydrogen, C₁-C₁₀ alkyl, and optionally a substituted acyl or an amino protecting group.

3. The small-molecule conjugate or a pharmaceutical salt thereof according to claim 1, wherein the Y is a chemotherapeutic drug comprising a pharmaceutically active compound and the drug may be linked to the B through an active group.

4. The small-molecule conjugate or a pharmaceutical salt thereof according to claim 1, wherein the Y is temsirolimus, an open-ring-cyclopropyl benzo[e]indolone analogue, pyrrolobenzodiazepine dimers, calichemicin, camptothecin and an analogue thereof, paclitaxel and a derivative thereof, vinblastine and an analogue thereof, dolastatins, auristatin, tubulysin, combretastatin, maytansine, DM1, epothilones, mitomycins, daunorubicin compounds, arenobufagin and a derivative thereof, or bufalin and a derivative thereof.

5. The small-molecule conjugate or a pharmaceutical salt thereof according to claim 4, wherein the Y is the open-ring-cyclopropyl benzo[e]indolone analogue, the pyrrolobenzodiazepine dimers, the calichemicin, the camptothecin, 7-ethyl-10-hydroxycamptothecin, exatecan and a derivative thereof, 7-cyclohexyl-21-fluorocamptothecin, DAVLBH, tubulysin B, MMAE, MMAF, an MMAF derivative, DM1, the paclitaxel and a derivative thereof, epothilone B, mitomycin C, the arenobufagin and a derivative thereof, the bufalin and a derivative thereof, the vincristine, daunorubicin, doxorubicin or epirubicin.

6. The small-molecule conjugate or a pharmaceutical salt thereof according to claim 2, wherein an A-B is selected from one of the following structures:

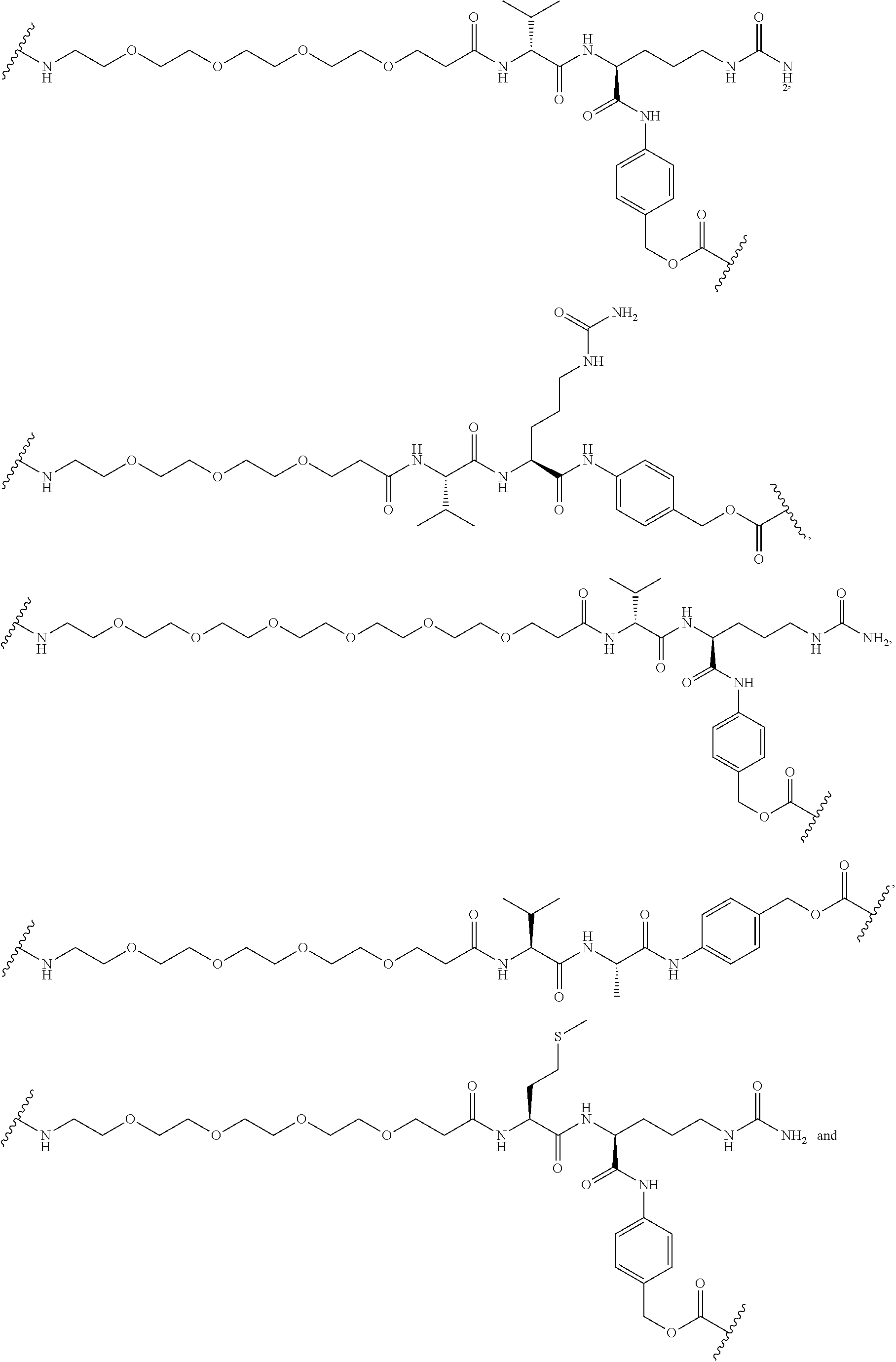

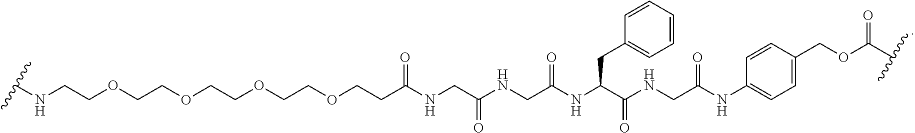 [SMILES: *NCCOCCOCCOCCOCCC(=O)NCC(=O)NCC(=O)N[C@@H](Cc1ccccc1)C(=O)NCC(=O)Nc1ccc(COC(*)=O)cc1]

7. The small-molecule conjugate or a pharmaceutical salt thereof according to claim 6, wherein the structure of the small-molecule conjugate is selected from one of the following structures: 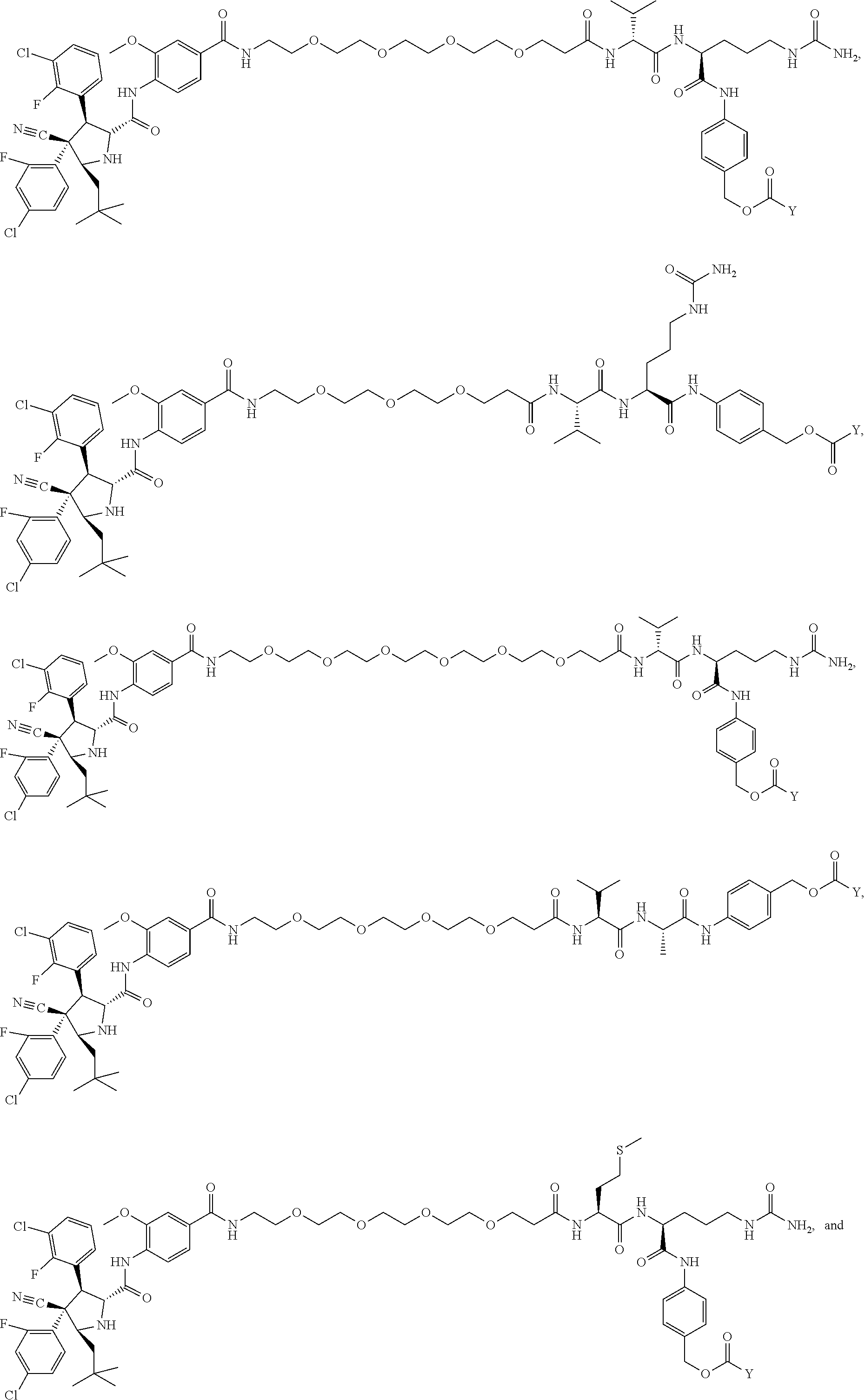 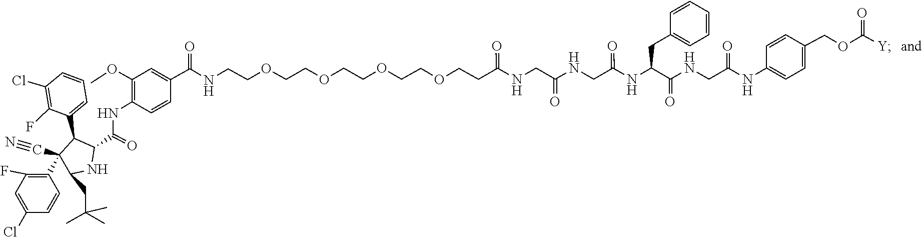
and the Y is selected from one of the following structures:

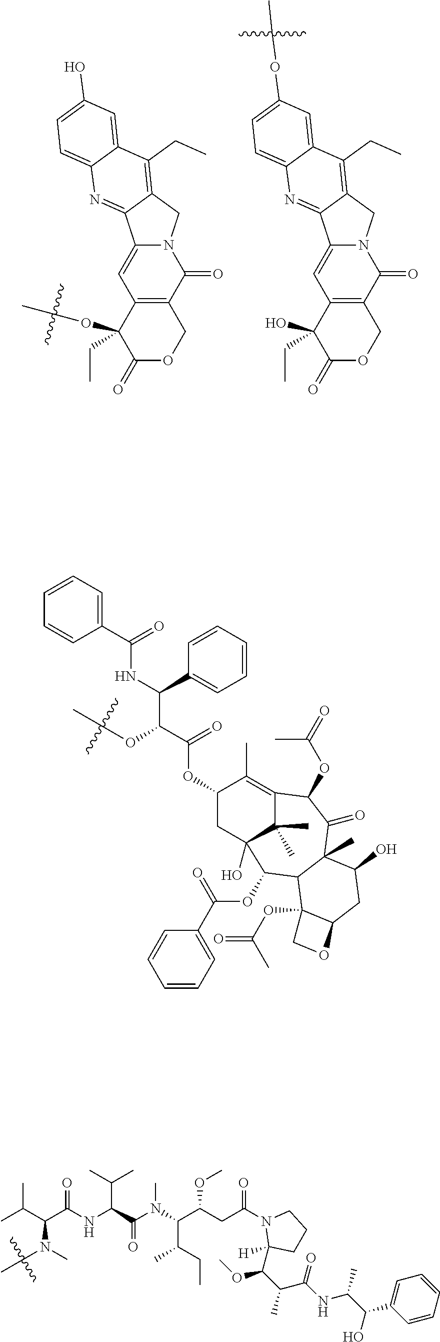

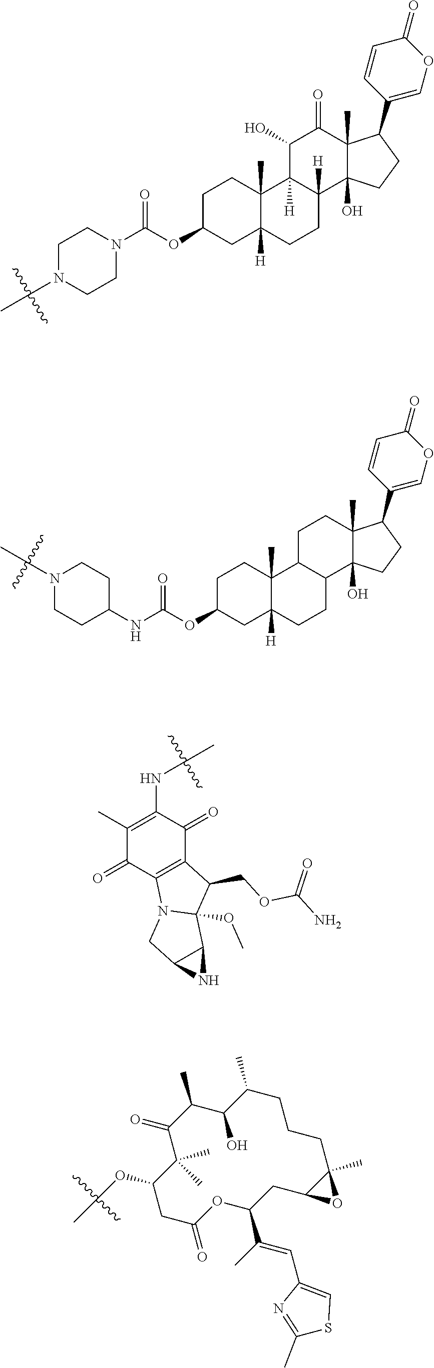

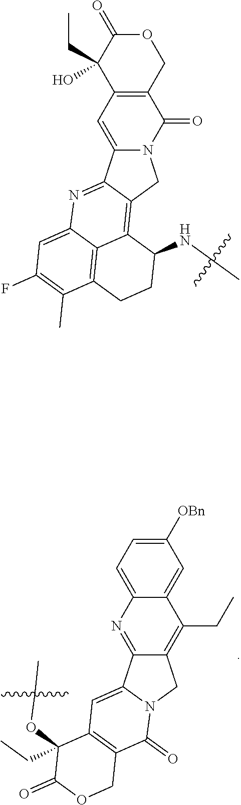 [SMILES: *N[C@H]1CCc2c(C)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccc(OCc3ccccc3)cc1c2CC]

8. The small-molecule conjugate or a pharmaceutical salt thereof according to claim 7, wherein the structure of the small-molecule conjugate is selected from one of the following structures:

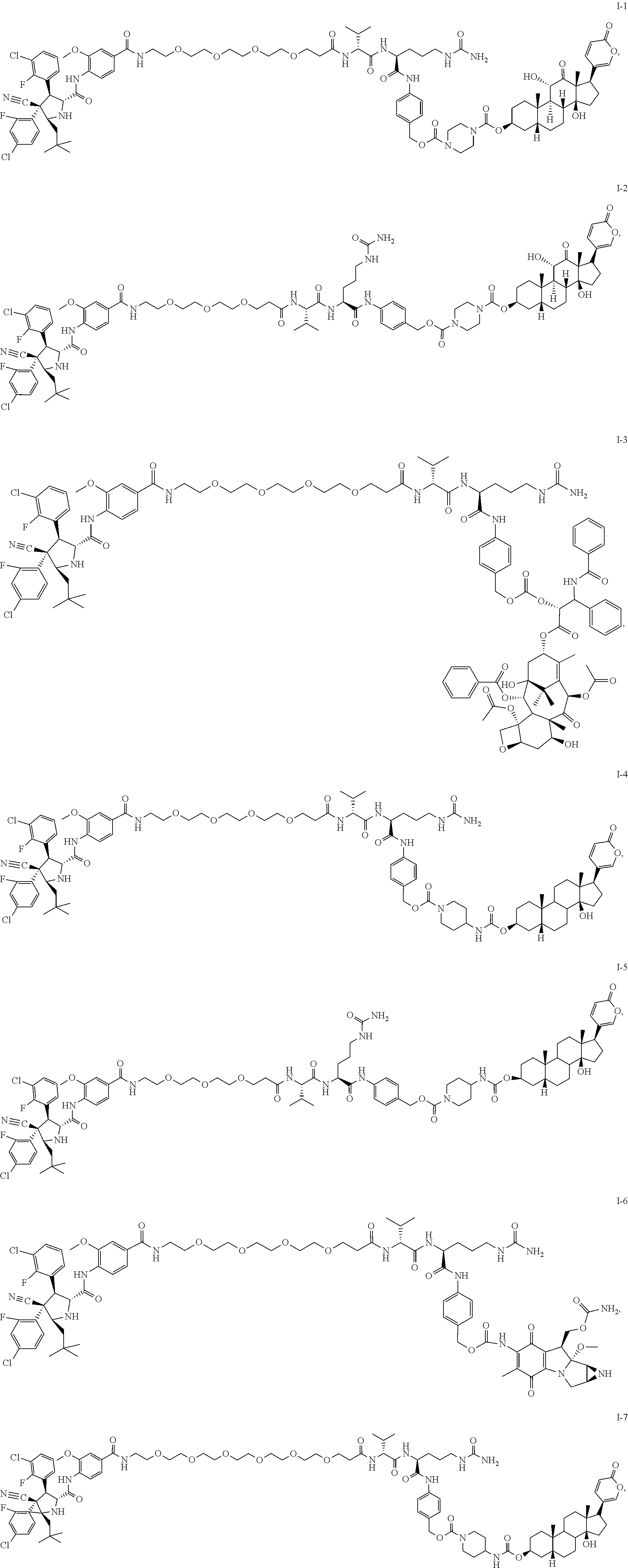

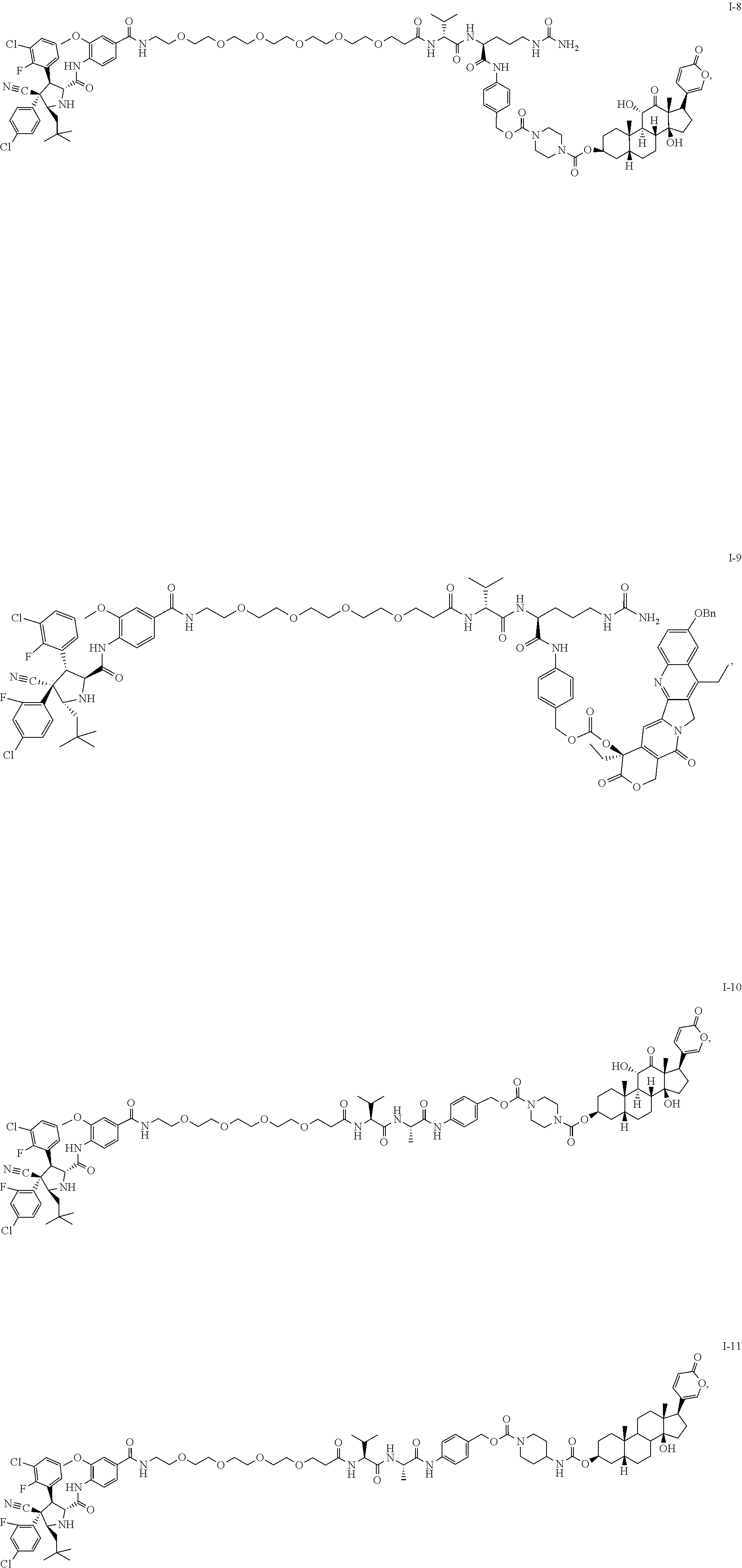

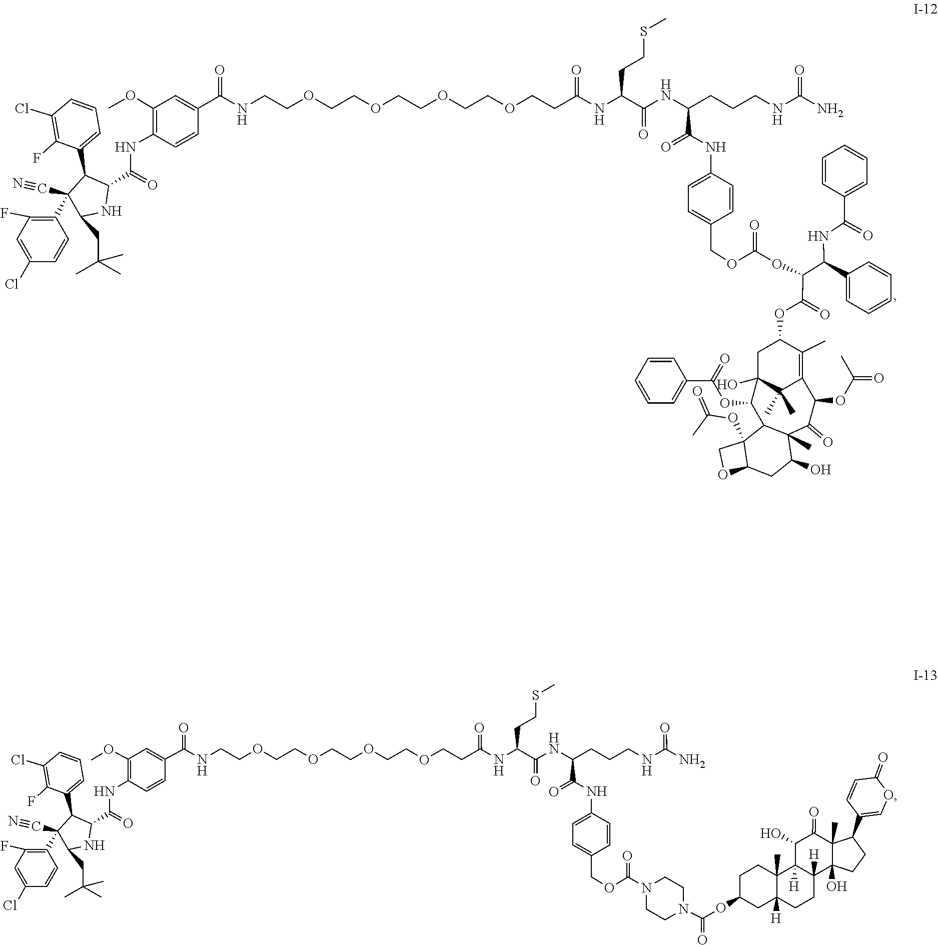

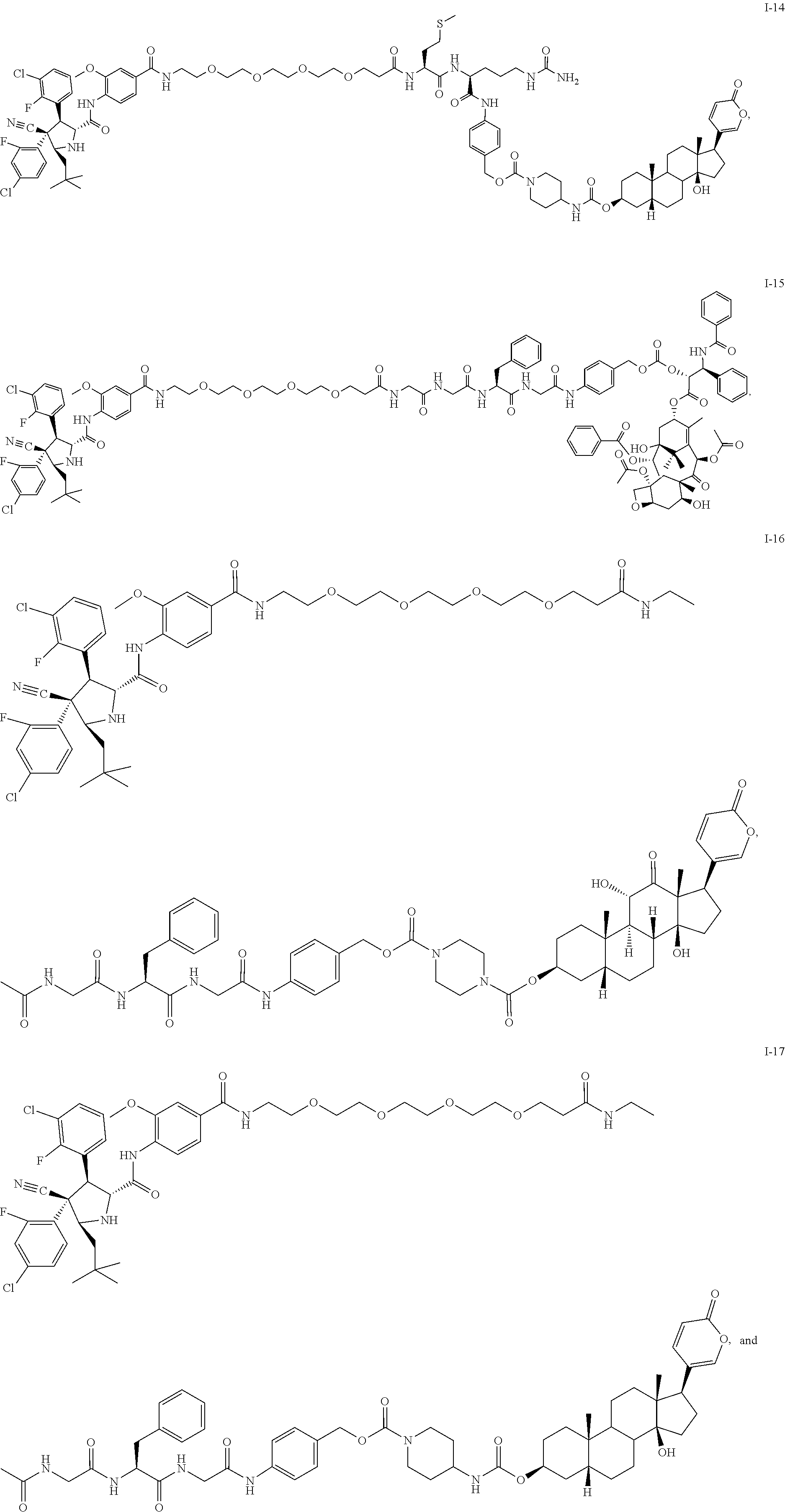

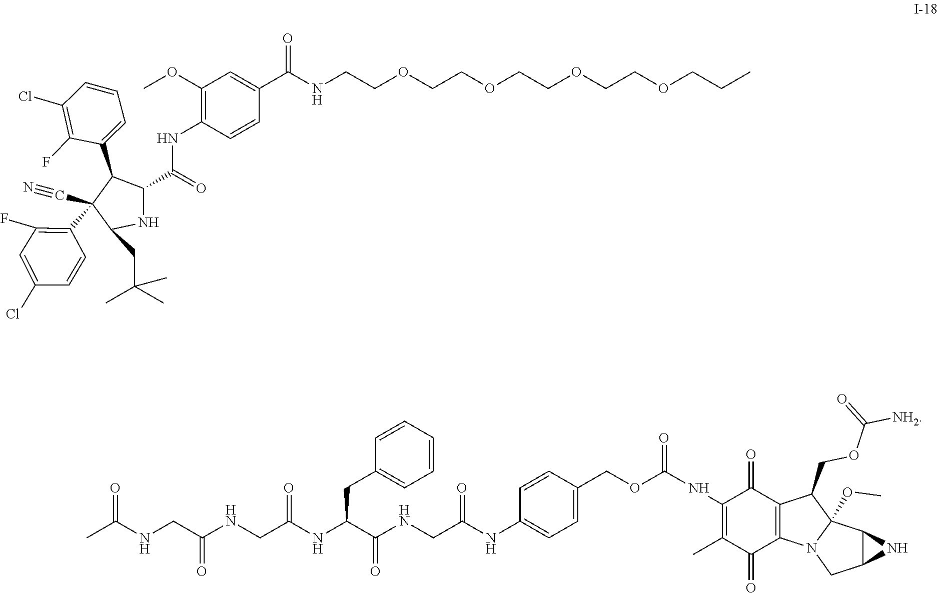

9. Use of the small-molecule conjugate or a pharmaceutical salt thereof according to claim 1 in the preparation of an antitumor drug, an anti-inflammatory drug, a drug for treating cardiovascular diseases, and a drug for resisting nervous system diseases.

10. A pharmaceutical composition comprising the small-molecule conjugate or a pharmaceutical salt thereof according to claim 1, wherein the small-molecule conjugate or the pharmaceutical salt thereof is used as a pharmaceutically active ingredient; or the pharmaceutical composition further comprises at least one therapeutic agent.

11. A pharmaceutical preparation comprising the small-molecule conjugate and a pharmaceutical salt thereof according to claim 1.

12. The use of the small-molecule conjugate or a pharmaceutical salt thereof of claim 9, wherein the B is selected from one or a combination of the following structures: 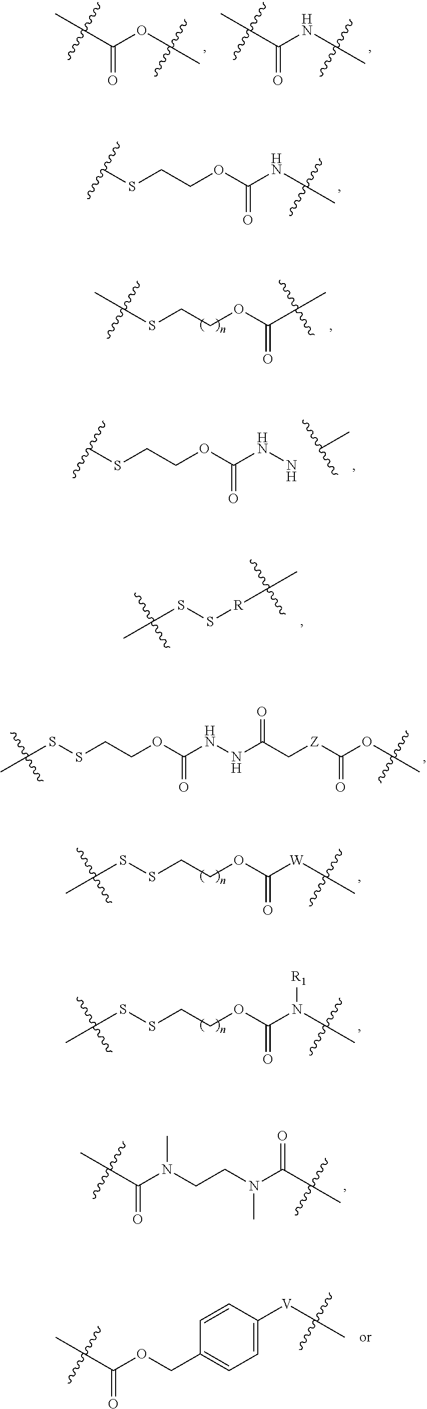 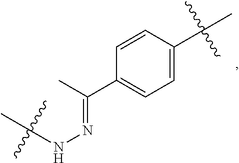
wherein n is 0, 1, 2, 3, or 4; R is —(CH₂)n— and —OOCCH₂—, and n is 1-10;
Z is —O—, —CH₂— or —NH—; W is O or S; and V is —NH—, —O—, or —S—; and
R₁ is hydrogen, C₁-C₁₀ alkyl, and optionally a substituted acyl or an amino protecting group.

13. The use of the small-molecule conjugate or a pharmaceutical salt thereof of claim 9, wherein the Y is a chemotherapeutic drug comprising a pharmaceutically active compound and the drug may be linked to the B through an active group.

14. The use of the small-molecule conjugate or a pharmaceutical salt thereof of claim 9, wherein the Y is temsirolimus, an open-ring-cyclopropyl benzo[e]indolone analogue, pyrrolobenzodiazepine dimers, calichemicin, camptothecin and an analogue thereof, paclitaxel and a derivative thereof, vinblastine and an analogue thereof, dolastatins, auristatin, tubulysin, combretastatin, maytansine, DM1, epothilones, mitomycins, daunorubicin compounds, arenobufagin and a derivative thereof, or bufalin and a derivative thereof.

15. The use of the small-molecule conjugate or a pharmaceutical salt thereof of claim 14, wherein the Y is the open-ring-cyclopropyl benzo[e]indolone analogue, the pyrrolobenzodiazepine dimers, the calichemicin, the camptothecin, 7-ethyl-10-hydroxycamptothecin, exatecan and a derivative thereof, 7-cyclohexyl-21-fluorocamptothecin, DAVLBH, tubulysin B, MMAE, MMAF, an MMAF derivative, DM1, the paclitaxel and a derivative thereof, epothilone B, mitomycin C, the arenobufagin and a derivative thereof, the bufalin and a derivative thereof, the vincristine, daunorubicin, doxorubicin or epirubicin.

16. The use of the small-molecule conjugate or a pharmaceutical salt thereof of claim 12, wherein an A-B is selected from one of the following structures:

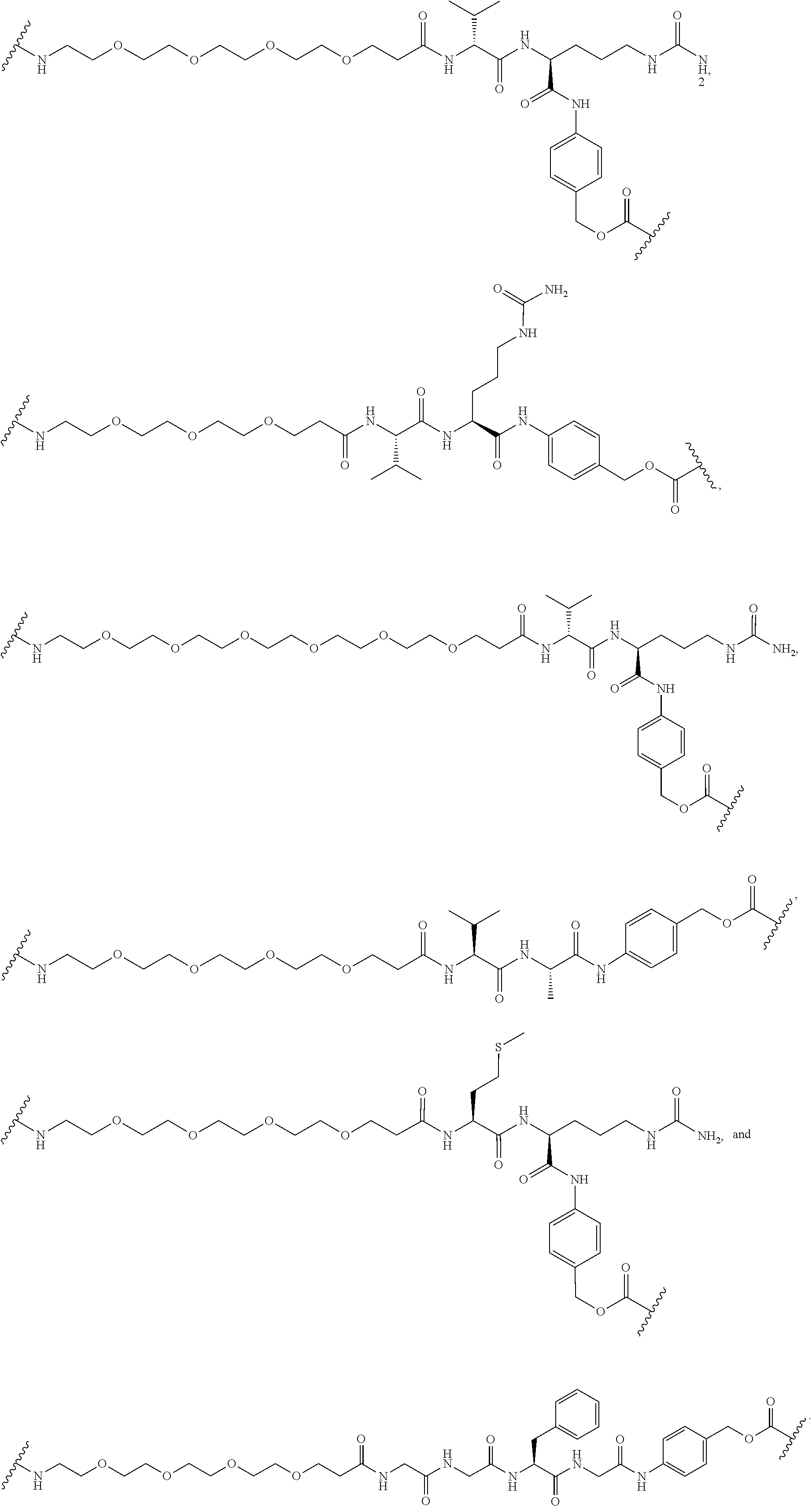

17. The use of the small-molecule conjugate or a pharmaceutical salt thereof of claim 16, wherein the structure of the small-molecule conjugate is selected from one of the following structures: 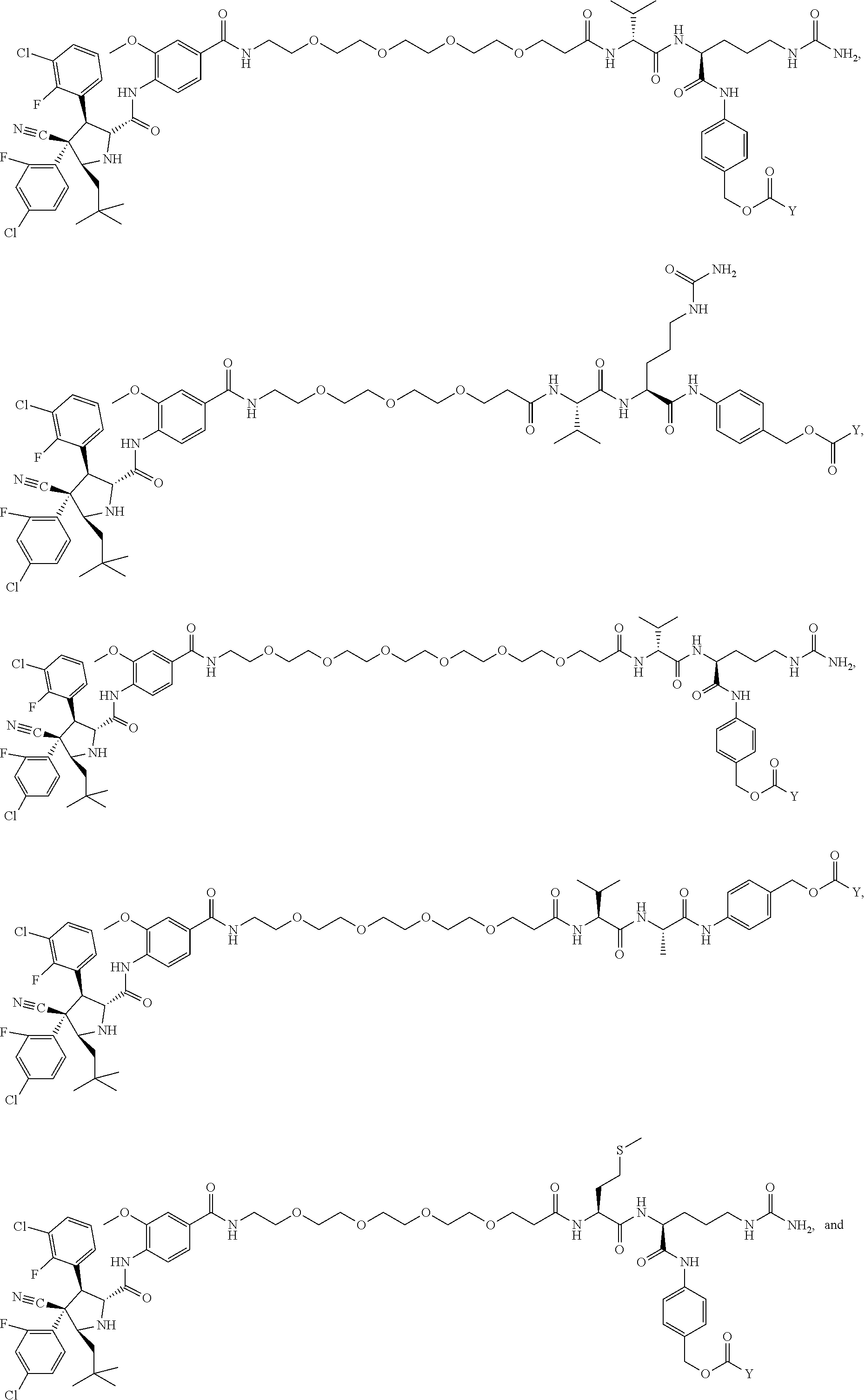 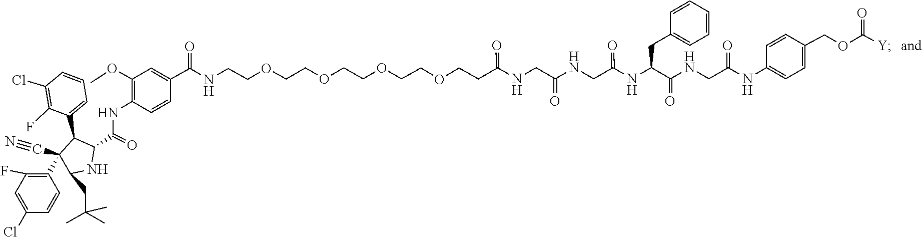
and the Y is selected from one of the following structures:

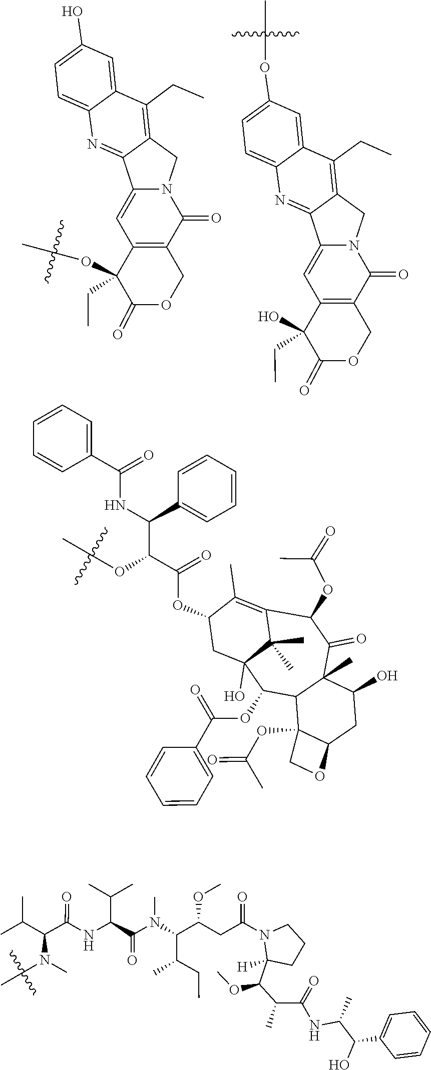

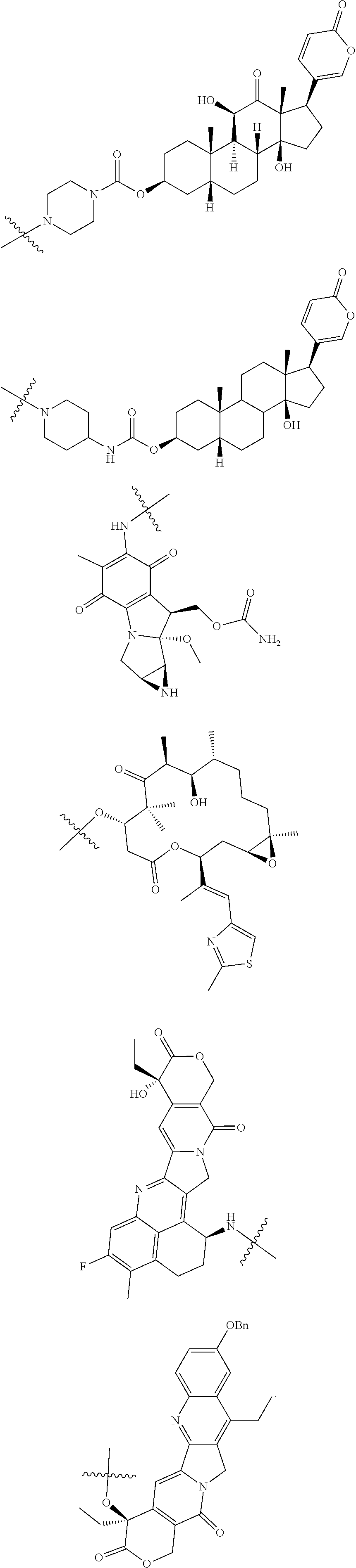

18. The use of the small-molecule conjugate or a pharmaceutical salt thereof of claim 17, wherein the structure of the small-molecule conjugate is selected from one of the following structures:

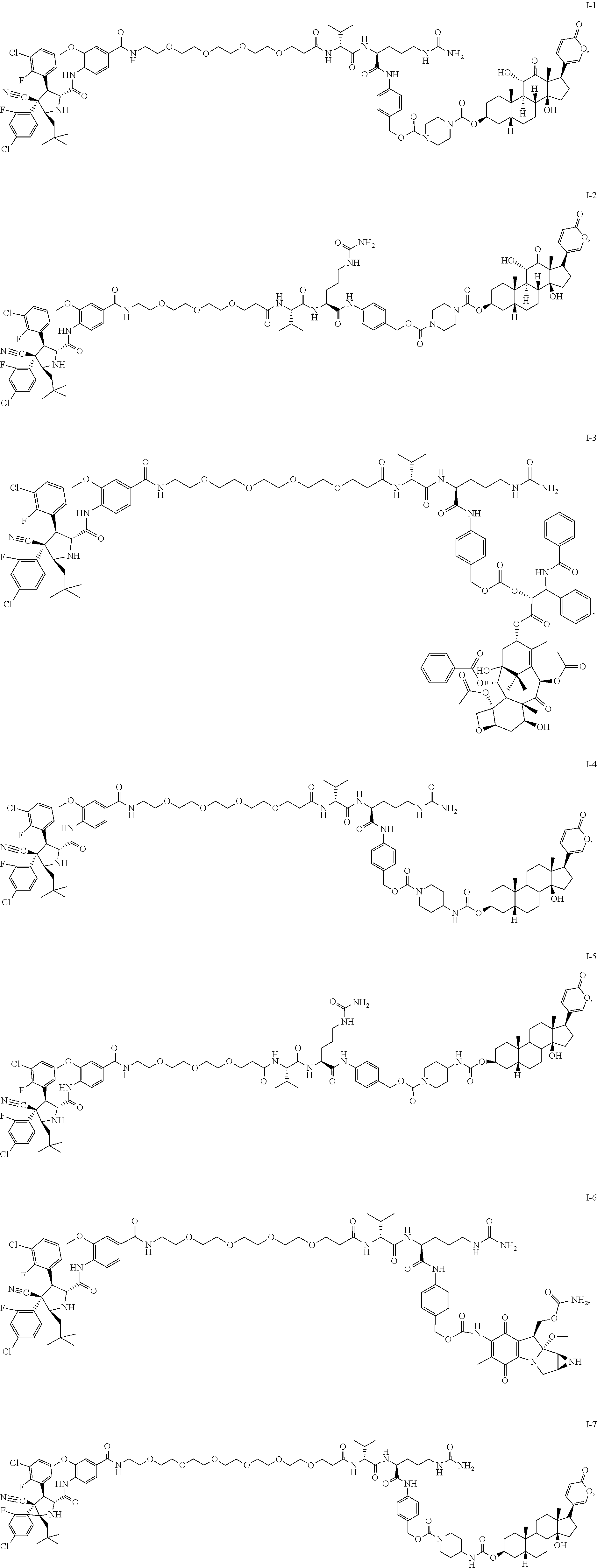

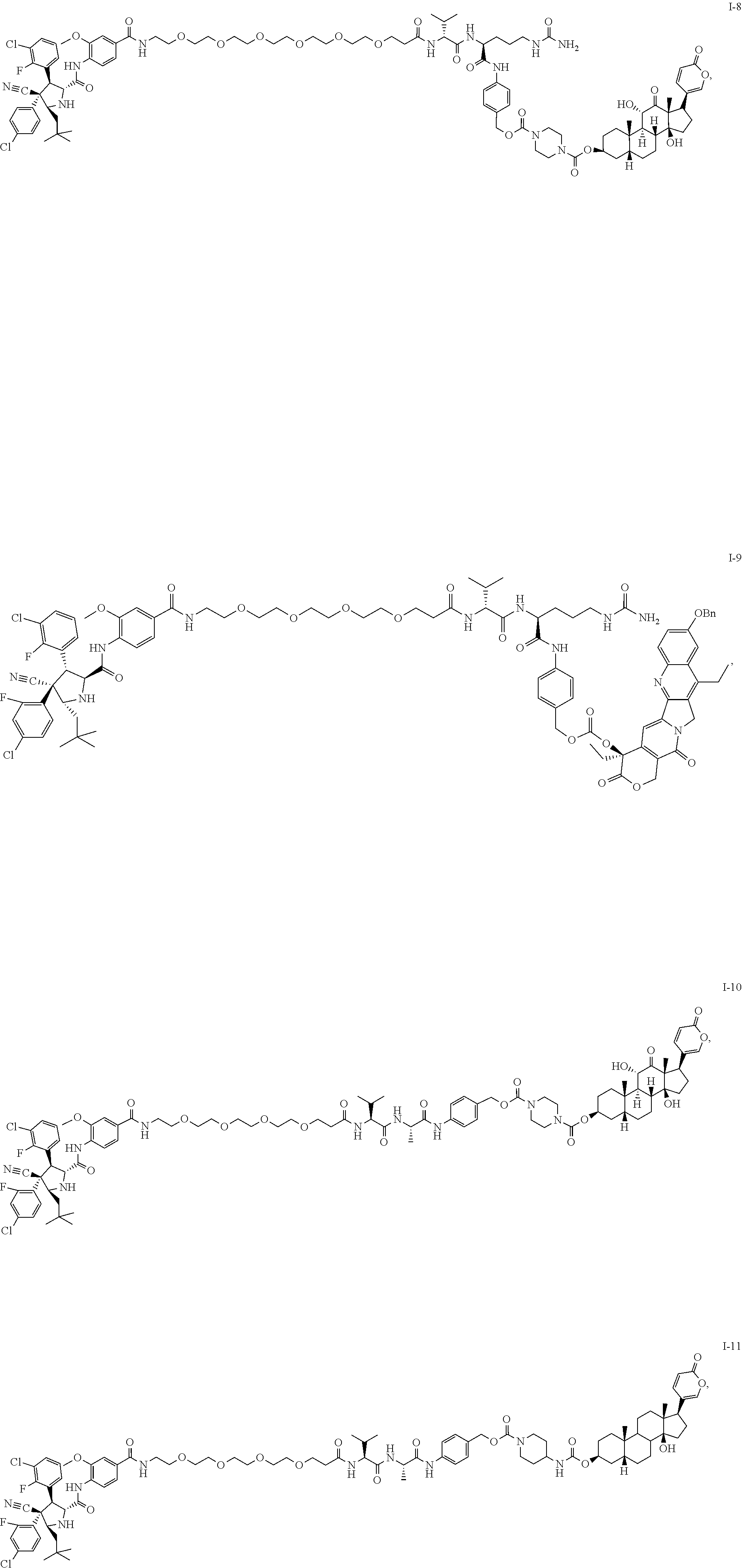

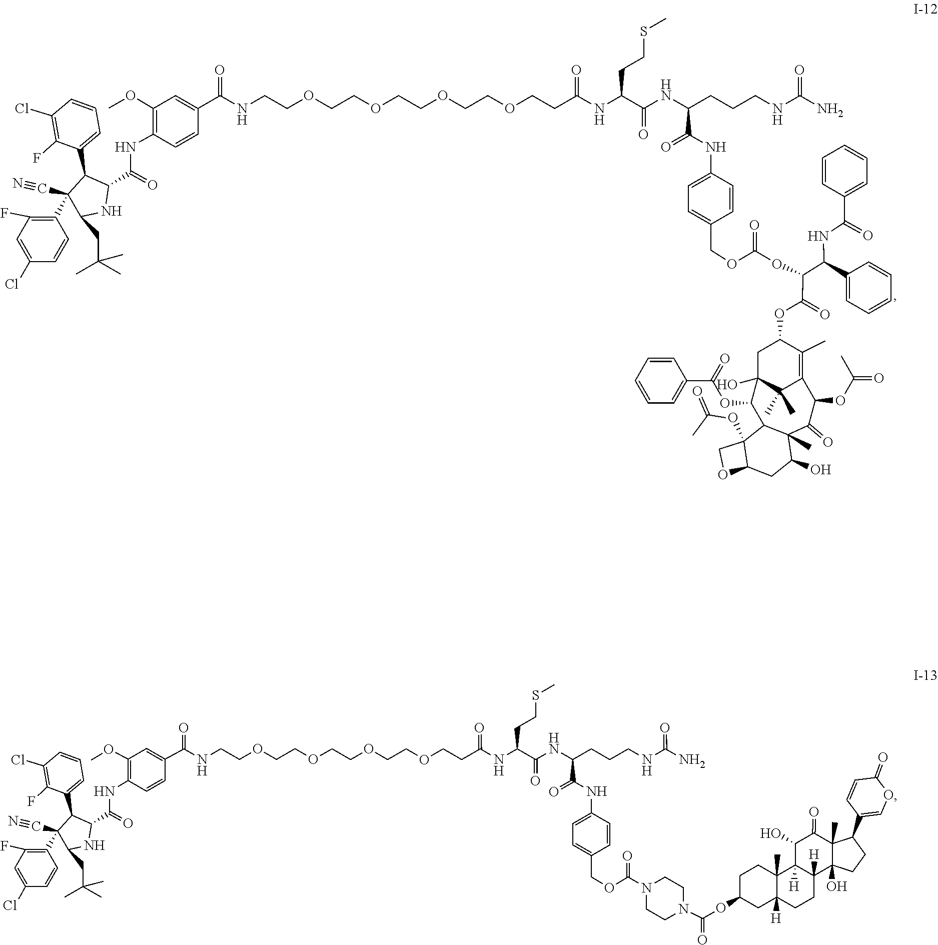

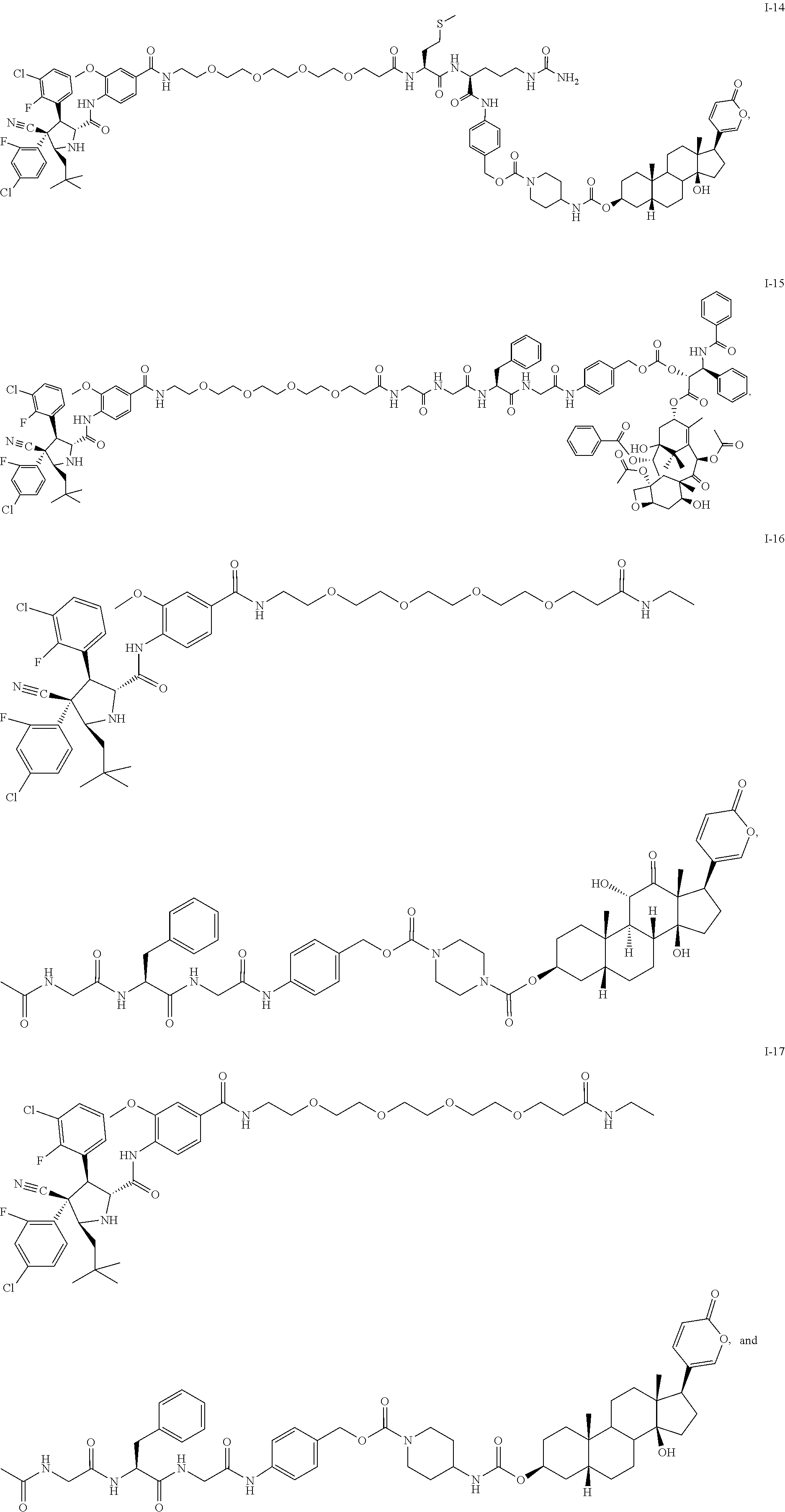

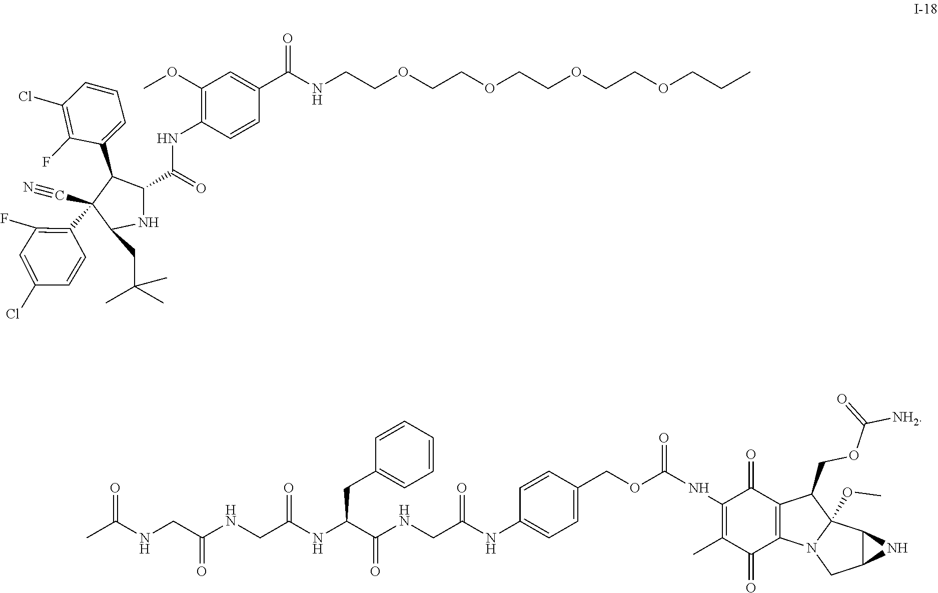

19. The pharmaceutical composition of claim 10, wherein the B is selected from one or a combination of the following structures: 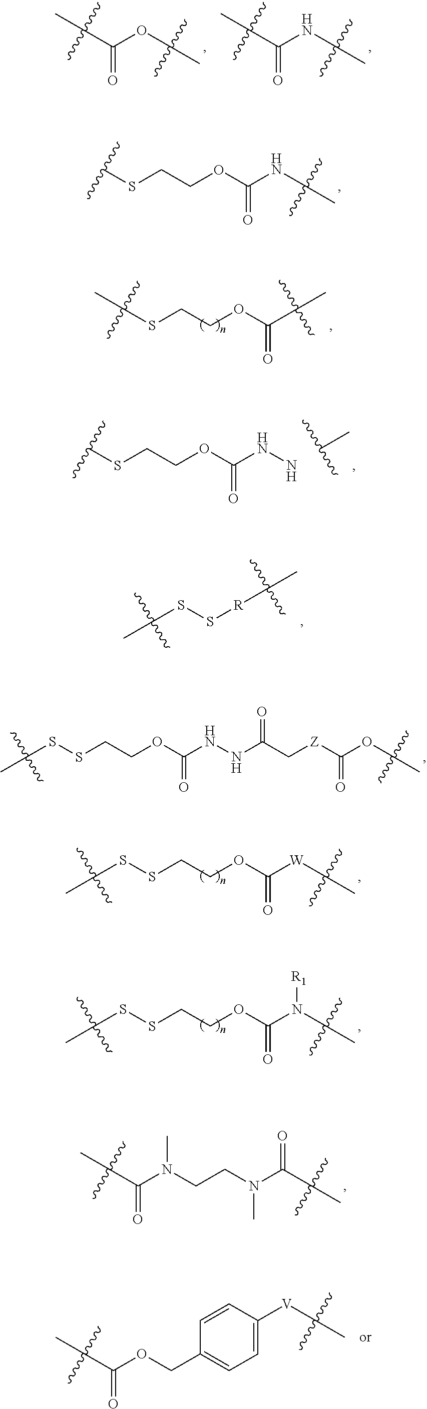 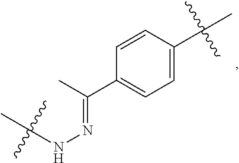
wherein n is 0, 1, 2, 3, or 4; R is —(CH₂)n— and —OOCCH₂—, and n is 1-10;
Z is —O—, —CH₂— or —NH—; W is O or S; and V is —NH—, —O—, or —S—; and
R₁ is hydrogen, C₁-C₁₀ alkyl, and optionally a substituted acyl or an amino protecting group.

20. The pharmaceutical composition of claim 10, wherein the Y is a chemotherapeutic drug comprising a pharmaceutically active compound and the drug may be linked to the B through an active group.
